(19) United States
(12) Patent Application Publication
(10) Pub. No.: US 2022/0315904 A1
(43) Pub. Date: Oct. 6, 2022

(54) MUTATION OF AN ACYL-COA SYNTHASE FOR INCREASED TRIACYLGLYCEROL PRODUCTION IN MICROALGAE

(71) Applicant: TOTALENERGIES SE, Courbevoie (FR)

(72) Inventors: Leonardo Magneschi, Edinburgh (GB); Elodie Billey, Fontaine (FR); Juliette Jouhet, Seyssinet-Pariset (FR); Eric Marechal, Grenoble (FR); Séverine Collin, La Buisse (FR)

(73) Assignee: TOTALENERGIES SE, Courbevoie (FR)

(21) Appl. No.: 17/615,856

(22) PCT Filed: Jun. 19, 2020
(86) PCT No.: PCT/EP2020/067114

§ 371 Date: Dec. 2, 2021

(30) Foreign Application Priority Data
Jun. 20, 2019 (EP) 19290043.9

(51) Int. Cl. C12N 9/10; C12P 7/6409; C12P 7/04; C12P 5/02

(52) U.S. Cl. CPC C12N 9/1029; C12Y 203/01086; C12P 7/6409; C12P 7/04; C12P 5/02

(57) ABSTRACT

The application generally relates to bioproduction of molecules of interest in microorganisms, more particularly in microalgae. In particular, the application relates to methods for increasing triacylglycerol production in micro-organisms, in particular in microalgae, using recombinant micro-organisms which have been genetically engineered to express or overexpress a mutant of a bubblegum-type acyl-CoA synthase, and uses thereof.

### TECHNICAL FIELD

The application generally relates to the bioproduction of molecules of interest in micro-organisms, particularly in microalgae. In particular, the application relates to methods and means for increasing triacylglycerol production in micro-organisms, in particular in microalgae.

### BACKGROUND

Microalgae have the ability to accumulate significant amounts of lipids, primarily in the form of triacylglycerol (TAG), especially under stress conditions like nutrient limitation, temperature, pH, or light stress (Lupette J et al. 2019, Algal Res 38:101415). The ability of microalgae to accumulate TAG has triggered their exploitation as host for fatty acid production, e.g. for biofuel production, for chemical applications or in food industry, such as for the industrial production of omega-3 polyunsaturated fatty acids.

For the biosynthesis of TAG and membrane glycerolipids, microalgae produce fatty acids (FA). Fatty acids are linear chains of carbon with a terminal carboxyl group. Prior to their incorporation into glycerolipids or other acyl-molecules, or prior their degradation by the beta-oxidation pathway, these fatty acids need to be activated, either by thioesterification to a coenzyme A (Acyl-CoA) in the cytosol of cells, or to an acyl carrier protein (Acyl-ACP) inside organelles (FIG. 1A, Section A).

Building blocks for the biosynthesis of glycerolipids consist therefore of a glycerol-3-phosphate (with 3 carbons numbered sn-1, sn-2 and sn-3), activated fatty acids in the form of Acyl-CoA or Acyl-ACP, and various polar head precursors (FIG. 1A, Section B). Membrane glycerolipids are composed of 1 glycerol backbone, 1 or 2 fatty acids and 1 polar head of a various nature (FIG. 1A, Section C). TAG are composed of 1 glycerol backbone and 3 fatty acids (FIG. 1A, Section D).

Exemplary microalgae of interest in the production of TAG are algae belonging to the Heterokont phylum (or Heterokonts), such as Nannochloropsis gaditana. Nannochloropsis species are a group of algae belonging to the Eustigmatophyte class, within the heterokont phylum. Nannochloropsis species are oleaginous, producing high levels of TAG, but the productivity has not yet reached industrial feasibility although much research efforts have been put into optimizing strains and culture conditions.

To increase TAG production, microalgae can activate two simultaneous synthetic processes: firstly, cells can simply synthesize new fatty acids, for the de novo production of TAG; secondly, they can recycle some of the fatty acids found in membrane lipids (particularly photosynthetic membranes lipids), which are then diverted towards TAG. Alternatively, TAG can also accumulate by preventing TAG catabolism. In this catabolic process, lipase hydrolysis releases free fatty acids, which are then activated into acyl-CoA and directed to the mitochondrion or the peroxisome where fatty acids are degraded.

Currently, the reduction of the availability of nitrate (NO₃⁻) in the growth medium is the most classical method used to naturally trigger the accumulation of TAG in microalgae (Adams et al., 2013, Bioresour Technol 131, 188-194). Nitrogen deprivation limits amino acid production and decreases protein synthesis, thereby impairing growth and photosynthesis, which leads to an accumulation of lipids, in particular TAG, which are used as carbon and energy provisions. Other methods rely on genetic modifications. For instance, disrupting the assimilation pathway of NO₃⁻ by genetic engineering has been considered as a way to trigger TAG accumulation, and reducing the expression of a nitrate reductase from P. tricornutum has been shown to promote TAG accumulation per cell (US20120282676). Other attempts to promote TAG accumulation include the stimulation of fatty acid and TAG biosynthesis, the blocking of pathways that divert carbon to alternative metabolic routes and eventually the arrest of TAG catabolism through genetic engineering of the microalgae (US20140256927).

Various species of the Nannochloropsis genus can be genetically modified (N. oculata, N. salina, N. oceanica, N. gaditana) and metabolic engineering methods have been implemented in order to increase TAG production (Iwai et al., 2015, Front Microbiol, 6: 912; Kang et al., 2015, Biotechnol Biofuels 8: 2000). None of these methods rely on the genetic mutation of an Acyl-CoA synthase (ACS) gene.

A large variety of fatty acids can be synthesized inside microalgae, but they are not equally incorporated in all kinds of glycerolipids. Some specific fluxes of fatty acids are directed to different end products, and ACS proteins are major actors in these channeling processes.

In heterokonts, specific fluxes of fatty acids include the two following major routes:
The initial fatty acids which are produced by fatty acid synthases contain 16 or 18 carbons and 0 or 1 double bond, e.g. palmitic acid (16:0, corresponding to 16 carbons and no double bonds). The 16:0 and 16:1 fatty acids are found in high levels in TAG of heterokonts.
In all heterokonts analyzed to date, including the eustigmatophyte Nannochloropsis and the diatoms Thalassiosira and Phaeodactylum, very long chain polyunsaturated fatty acids (VLC-PUFAs), especially eicosapentaenoic acid (EPA, 20:5 with 20 carbons and 5 double bonds) and docosahexaenoic acid (DHA, 22:6) are also produced, after a series of elongations and desaturations from linolenic acid (18:3). VLC-PUFAs are overrepresented in chloroplast membrane glycerolipids like monogalactosyldiacylglycerol (MGDG), digalactosyldiacylglycerol (DGDG), sulfoquinovosyldiacylglycerol (SQDG), or endoplasmic reticulum membrane glycerolipids like phosphatidylcholine (PC) and diacylglyceryltrimethylhomoalanine DGTA in Phaeodactylum (Abida et al., 2015) and MGDG, DGDG, PG, phosphatidylethanolamine (PE) and diacylglyceryltrimethylhomoserine (DGTS) in Nannochloropsis (Alboresi A. et al. 2016, Plant Physiol. 171(4):2468-82). In general, VLC-PUFAs are poorly present in TAG of these microalgae.

There is a need for alternative methods for enhancing triacylglycerol accumulation in microalgae, preferably without compromising cell growth and biomass yield so as to improve overall lipid productivity.

### SUMMARY OF THE INVENTION

The present invention is based, at least in part, on the finding that the genetic modification in microalgae of an acyl-CoA synthase (ACS) isoform, particularly belonging to the Bubblegum subfamily (ACSBG), results in a redirection of fatty acids toward the production of TAG, with the microalgae expressing the mutated ACSBG exhibiting a decrease of some fatty acids in membrane lipids and an increase in TAG content under non-starved conditions.

The invention thus provides methods for the increased production of one or more molecules of interest of the lipid metabolic pathway in a micro-organism, which methods comprise providing a recombinant micro-organism which has been genetically engineered to comprise a polynucleotide encoding a mutant acyl-CoA synthase and culturing said recombinant micro-organism thereby allowing the production of said molecule of interest. In particular embodiments the methods comprise providing a recombinant micro-organism expressing a mutant of an acyl-CoA synthase gene of the Bubblegum type (ACSBG), culturing said recombinant micro-organism thereby allowing the production of said one or more molecule of interest; and, optionally recovering said one or more molecules of interest. In particular embodiments, the molecules of interest are triacylglycerols, fatty acids, hydrocarbons or fatty alcohols, preferably triacylglycerols. In particular embodiments, the triacylglycerol content in said recombinant micro-organism is at least 150% of the triacylglycerol content of a corresponding micro-organism which does not comprise said mutant of an acyl-CoA synthase gene.

In particular embodiments of the methods described herein, the micro-organism is a microalga. More particularly, the microalga is selected from the Heterokonta phylum. Most particularly, the microalga is selected from the Bacillariophycea or Eustigmatophyceae, preferably wherein the microalga is selected from the Nannochloropsis genus or Phaeodactylum genus.

In particular embodiments, the acyl-CoA synthase of the Bubblegum-type has an amino acid sequence set forth in SEQ ID NO:7 or 8, or a homolog thereof having the Motif II sequence set forth in SEQ ID NO:1. In further embodiments, said homolog has an amino acid sequence set forth in SEQ ID NO:9-14 or 22-28, preferably an amino acid sequence set forth in SEQ ID NO:9-14.

In particular embodiments, the mutated gene encodes a functional acyl-CoA synthase of the Bubblegum type (i.e. the mutant Bubblegum-type acyl-CoA synthase retains at least part of the activity of the wild-type acyl-CoA synthase).

In particular embodiments of the methods described herein, the mutant of an acyl-CoA synthase of the Bubblegum-type is a mutant of a Bubblegum-type acyl-CoA synthase from a Heterokonta species. In particular embodiments, said mutant of an acyl-CoA synthase of the Bubblegum-type is a protein comprising 1-5 amino acid substitutions, deletions or additions in a region corresponding to the region spanning from amino acid 82 to 120 of amino acid sequence SEQ ID NO. 7. More particularly, the inventors have found that the domain spanning from amino acid 83 to 102 of SEQ ID NO:7, i.e. corresponding to SEQ ID NO: 17, such as in amino acids 96-98 of SEQ ID NO:7, is of particular interest to generate ACSBG mutations. In particular embodiments, said mutant of an acyl-CoA synthase of the Bubblegum-type is a protein comprising one or more amino acid substitutions and/or additions in the region between amino acids 96 and 98 of the amino acid sequence set forth in SEQ ID NO:7 or in a region corresponding to the region between amino acids 96 and 98 of the amino acid sequence set forth in SEQ ID NO:7. In further particular embodiments, said mutant of an acyl-CoA synthase of the Bubblegum type has an amino acid sequence set forth in SEQ ID NO:15, 16 or 44 or comprises the corresponding mutations of SEQ ID NO:15, 16 or 44 with respect to SEQ ID NO:7.

In further particular embodiments, the mutant of an acyl-CoA synthase is a mutant of a Nannochloropsis or Phaeodactylum bubblegum-type acyl-CoA synthase. In yet further particular embodiments, the mutant of an acyl-CoA synthase is a mutant of the Bubblegum-type acyl-CoA synthase set forth in SEQ ID NO:7 or 8.

In particular embodiments, the methods comprise introducing a mutation in an endogenous gene encoding for the acyl-CoA synthase of the Bubblegum-type of said micro-organism.

A further aspect of the invention provides recombinant micro-organisms, preferably recombinant microalga, comprising a polynucleotide sequence encoding a mutated acyl-CoA synthase protein of the Bubblegum type, wherein said protein comprises 1-5 amino acid substitutions, deletions or additions in a region corresponding to the region spanning from amino acid 83 to 102 of amino acid sequence SEQ ID NO. 7. In embodiments, said protein comprises one or more amino acid substitutions and/or additions in amino acids 96-98 of the amino acid sequence SEQ ID NO. 7 or in a region corresponding to amino acids 96-98 of the amino acid sequence SEQ ID NO. 7 In particular embodiments, the mutated acyl-CoA is a mutated form of the Nannochloropsis gaditana bubblegum-type acyl-CoA synthase having amino acid sequence of SEQ ID NO. 7, or a muted form of the Phaeodactylum tricornutum bubblegum-type acyl-CoA synthase having an amino acid sequence of SEQ ID NO. 8. In further particular embodiments, the mutated acyl-CoA synthase protein comprises the corresponding mutations of SEQ ID NO. 15, SEQ ID NO. 16 or SEQ ID NO. 44 with respect to the wildtype SEQ ID NO. 7. In yet further embodiments, the mutated protein corresponds to SEQ ID NO: 15, 16, or 44.

The invention also provides vectors comprising one or more polynucleotides encoding a mutated acyl-CoA synthase of the Bubblegum-type wherein said mutated acyl-CoA synthase comprises 1-5 amino acid substitutions, deletions or additions in a region corresponding to the region spanning from amino acid 83 and 102 of amino acid sequence SEQ ID NO. 7.

The invention also provides the use of a recombinant micro-organism as described herein, for the production of molecules of the lipid metabolic pathway, preferably for the production of triacylglycerols, fatty acids, hydrocarbons or fatty alcohols.

### BRIEF DESCRIPTION OF THE FIGURES

The teaching of the application is illustrated by the following Figures which are to be considered as illustrative only and do not in any way limit the scope of the claims.

FIG. 1A: Overview of glycerolipid biosynthesis. This scheme shows fatty acid as grey bars. (section A) Prior to their incorporation inside glycerolipids, fatty acids need to be activated, either by thioesterification to co-enzyme A, forming Acyl-CoA, or by thioesterification to acyl carrier protein, forming Acyl-ACP. (section B) The precursors consist therefore of a glycerol-3-phosphate, activated fatty acids (Acyl-CoA or Acyl-ACP) and polar head precursors. Glycerol-3-phosphate is made of a 3-carbon backbone, each being numbered sn-1, sn-2 and sn-3. The sn-1 and sn-2 carbon harbor a hydroxyl group (—OH), whereas the sn-3 position is linked to a phosphate group (P). (section C) Membrane glycerolipids contain one or two fatty acids. When the polar head contains a phosphate group, like phosphatidylcholine, phosphatidylethanolamine, etc., these glycerolipids are called phospholipids. When the polar head contains galactosyl residues, they are called galactolipids. Diacylglycerol is the simplest membrane glycerolipid structure, with a hydroxyl group at the sn-3 position. (section D) When the polar head is replaced by a third fatty acyl, a triacylglycerol is formed. The enzymatic activity corresponding the “acyl-CoA synthesis” or ACS is indicated.

FIG. 1B: Overview of Acyl-CoA functionality (adapted from Coleman et al., 2002, J Nutr 132, 2123-2126). Acyl-CoA can be used (1) for de novo synthesis of glycerolipids, firstly phosphatidic acid, then diacylglycerol and other membrane glycerolipids. Some specific acyl-CoA, such as 18:3-CoA can also (2) be channeled toward fatty acid desaturation and elongation (dashed lines), thus producing very-long chain polyunsaturated fatty acids (VLC-PUFAs) that are incorporated inside membrane glycerolipids. Acyl-CoA can be (3) incorporated into diacylglycerol and/or membrane lipids to form TAG. Acyl-CoA can be used for the synthesis of other acyl-molecules, such as (4) sphingolipids, (5) acylated proteins, (6) cholesterol esters, (7) signaling molecules. Eventually, Acyl-CoA can be (8) degraded via the beta-oxidation pathway occurring in mitochondria and/or peroxisomes, or be converted into ketones.

FIG. 2. Specific motif characterizing Acyl-CoA synthases of the Bubblegum family as defined by (Steinberg et al., 2000), by aligning amino acid sequences/fragments from ACSBG in Nannochloropsis gaditana, Phaeodatylum tricornutum, Gallus gallus or chicken (GgACSBG1 and 2), Homo sapiens (HsACBG1 and 2), Mus musculus or mouse (mmBG) and Drosophila melanogaster (dmBG and dmBG-H1). Identical residues in all sequences are shown in black boxes; residues conserved in more than 50% of the sequences are shown in bold. This motif is also called “Motif II”.

FIG. 3: Phylogenetic tree of ACSBG proteins of Nannochloropsis (Naga_100014 g59 (NgACSBG); Naga_100012 g66; Naga_101051 g1; Naga_100047 g8; Naga_100649 g1 and Naga_100035 g43), Phaeodactylum (Phatr3_J20143 (ACS1), Phatr3_J12420 (ACS2), Phatr3_J54151 (ACS3), Phatr3_J45510 (PtACSBG or ACS4) and Phatr3_J17720 (ACL1)), i.e. Drosophila melanogaster (DmACSBGa, genbank reference NP_524698; DmACSBGc, genbank reference NP_001285923); Homo sapiens (HsACBG1, Uniprot reference Q96GR; HsACBG2, Uniprot reference Q5FVE4); Mus musculus or mouse (MmACSBG1, genbank reference NP_444408); Gallus gallus or chicken (GgACSBG1, Uniprot reference F1NLD6; GgACSBG2, genbank reference XP_015155301).

FIG. 4: schematic representation of pCT61 and pCT62 vectors used for generation of NgACS mutants in Nannochloropsis via genome editing (TALE-N).

FIG. 5: screening of TALE-N driven edition in ACSBG mutants. A. T7E1 assay on target genomic sequence showing occurrence of mutations in colony 11. This mutant population was sub-cloned onto a new selective plate and colonies arising from single cells were re-sequenced for the ACSBG locus. B. Validation of genome editing in the Tal2.11VSC #31 and Tal2.11VSC #5 clones, in which insertion and/or replacement of base pairs (shown in black) inside the FokI activity region (represented as a square) resulted in the appearance of novel codons

FIG. 6: Point mutations obtained in NgACSBG #5 and in NgACSBG #40 endogenous protein sequences, TAL2.11vsc #5 (SEQ ID NO. 15) and TAL2.11vsc #40 (SEQ ID NO. 16), following specific TALE-N nucleotidic modifications in Nannochloropsis gaditana genome, compared to the wild type Nannochloropsis gaditana ACSBG (SEQ ID NO. 7). In grey box, wild type amino acids encoded at the level of TALE-N nucleotidic target; in black boxes, inserted mutations.

FIG. 7: A. Growth of Nannochloropsis WT, Empty vector control (EV) and clones TAL2.11vsc #55 and TAL2.11vsc #40 in 50 ml culture in replete ESAW medium flask. B. TAG content of Nannochloropsis WT, Empty vector control (EV) and clones TAL2.11vsc #5 and TAL2.11vsc #40 in 50 ml culture flask.

FIG. 8: A. Growth of Nannochloropsis WT, Empty vector control (EV) and clone TAL2.11VSC #5 in CO₂ enriched air lift photobioreactor (PBR) in replete ESAW medium. B. TAG content of Nannochloropsis WT, Empty vector control (EV) and clone TAL2.11VSC #5

FIG. 9: Nucleic acid sequence of the pCT61 vector. NotI and HindIII sites used for subcloning are highlighted in bold, whereas Transcription Activator-like effector nuclease (TALEN) subunits are underlined.

FIG. 10: Nucleic acid sequence of the pCT62 vector. NotI and HindIII sites used for subcloning are highlighted in bold, whereas TALEN subunits are underlined.

FIG. 11: Amino acid sequences of bubblegum-type acyl-CoA synthase homologs.

FIG. 12: Presentation of the secondary structure of NgACSBG as predicted by the GORIV (Garnier-Osguthorpe-Robson-IV) secondary prediction method (https://npsa-prabi.ibcp.fr/cgi-bin/npsa_automat.pl?page=npsa_gor4.html on Mar. 29, 2019, with default parameters). GOR4 secondary structures are indicated as follows: (h): alpha helix; (e): extended strand; (c): random coil. The region of interest for mutation preferably spans between the two highlighted alpha-helices.

FIG. 13: Multiple alignment of ACSBG proteins sequences from animals and Heterokonts. Amino acid sequences from ACSBG in Nannochloropsis gaditana (NgACBG) (SEQ ID NO:7), Phaeodatylum tricornutum (PtACSBG) (SEQ ID NO:8), Gallus gallus or chicken (GgACSBG1 and 2) (SEQ ID NO:22 and 23), Homo sapiens (HsACSBG1 and 2) (SEQ ID NO: 25 and 24), Mus musculus or mouse (MmACSBG1) (SEQ ID NO:26) and Drosophila melanogaster (DmACSBGa and c) (SEQ ID NO: 27 and 28) were aligned using the Multalin method. Amino acid positions conserved in more than 80% sequences are shown in black boxes; residues conserved in more than 50% sequences are shown in bold. Consensus highlights positions were residues are conserved (capital letter corresponding to the amino acid 1-letter code and the following symbol for amino acids with similar structures: T for I or V; ‘$’ for L or M; ‘%’ for F or Y and ‘#’ for N, D, Q or E. Motifs I, II, III, IV and V are conserved in all sequences. Position of point mutations in the Nannochloropsis gaditana sequence, introduced using the TALE-N strategy, are shown by stars.

FIG. 14: NgACSBG mutants. (A) Mutant selection. Following N. gaditana co-transformation with the two specifically designed TALE-N subunits, obtained clones were analyzed by treatment with a T7 endonuclease, to detect cleaved DNA at mismatched positions and assess occurrence of genome editing at the target locus. When this test allowed detecting a mutation, the cell population was sub-cloned onto a new selective plate and colonies arising from single cells were sequenced at the NgACSBG, inside the FokI activity region (frame) resulting in the appearance of novel codons. Three mutant lines harboring mutated endogenous NgACSBG are shown, called here NgACSBG #5, NgACSBG #31 and NgACSBG #40. Only two to three amino acids were modified in the endogenous proteins. Nucleotide sequences of (mutated) NgACSBG around and inside the FokI activity region are shown for NgACSBG (SEQ ID NO:46), NgACSBG #5 (SEQ ID NO:47), NgACSBG #31 (SEQ ID NO:45) and NgACSBG #40 (SEQ ID NO:48) (B) Point mutations in NgACSBG #5 (SEQ ID NO:15), NgACSBG #31 (SEQ ID NO:44) and NgACSBG #40 (SEQ ID NO:16) compared to the wild type Nannochloropsis gaditana ACSBG (SEQ ID NO. 7). In grey boxes, wild type (WT) residues at the TALE-N nucleotidic target; in black boxes, inserted mutations.

FIG. 15: Compared analysis of NgACSBG #5 and NgACSBG #31 with untransformed wild type cells (WT) and cells transformed with an empty vector (EV). (A) Growth was measured by cell counting as described Example 2 at day 3, 7 and 12 following inoculation (D3, D7 and D7, respectively). (B) Fatty acid content per million cells at D3, D7 and D12 following inoculation. (C) Triacylglycerol content, in mol % of total glycerolipids, at D3, D7 and D12 following inoculation. (D) Fatty acid profile at D3. Total FAs were extracted and, following methanolysis, obtained FA methyl esters were analyzed and quantified by gas chromatography coupled with ion flame detection (GC-FID), as described in Example 4. (E) Glycerolipid profile at D3. Each glycerolipid class was analyzed by liquid chromatography coupled to tandem mass spectrometry (LC-MSMS) and quantified as described in Example 4. DAG, diacylglycerol; DGDG, diacylglycerol; DGTS, diacylglyceryl-N,N,N-trimethylhomoserine; FA, fatty acid; MGDG, monogalactosyldiacylglycerol; PC, phosphatidylcholine; PE, phosphatidylethanolamine; PG, phosphatidylglycerol; PI, phosphatidylinositol; SQDG, sulfoquinovosyldiacylglycerol; TAG, triacylglycerol. Data are the average of 4 replicates. Error bars, standard deviation. (*), P-value <0.05; student's t-test using WT as a reference.

FIG. 16: 3D model of NgACSBG bound to CoA, AMP and α-linolenic acid (18:3). Four models of NgACSBG were obtained based on similarity with acetyl- and acyl-CoA synthetase of known 3D structure, as described in Example 5. The Mcons1 model of NgACSBG is shown with the visualization program VMD (Humphrey et al., 1996. J Mol Graph 14: 33-38, 27-38). The backbone of residues around AMP and CoA, listed in Tables 3 and 4, are shown in licorice. The IGF triad is represented (196, G97, F98). The conserved Y216-K225 active site is shown in licorice mode. The position of IGF close to 18:3 is similar in all four NgACSBG models. Abbreviations: COA, Coenzyme A; AMP, adenosine monophosphate; LIP, α-linolenic acid.

### DETAILED DESCRIPTION OF THE INVENTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. Where reference is made to embodiments as comprising certain elements or steps, this encompasses also embodiments which consist essentially of the recited elements or steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +1-0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

As used herein, the terms “microbial”, “microbial organism” or “micro-organism” are intended to mean any organism that exists as a microscopic cell that is included within the domains of archaea, bacteria or eukaryotes. Therefore, the term is intended to encompass prokaryotic or eukaryotic cells or organisms having a microscopic size and includes bacteria, archaea and eubacteria such as cyanobacteria of all species as well as eukaryotic micro-organisms such as fungi, including yeasts, and algae. The term also includes cell cultures of any species.

The term “microalga” or “microalgae” (plural) as used herein refers to microscopic alga(e). “Microalgae” encompass, without limitation, organisms within (i) several eukaryotic phyla, including the Rhodophyta (red algae), Chlorophyta (green algae), Dinoflagellata, Haptophyta, (ii) several classes from the eukaryotic phylum Heterokontophyta which includes, without limitation, the classes Bacillariophycea (diatoms), Eustigmatophycea, Phaeophyceae (brown algae), Xanthophyceae (yellow-green algae) and Chrysophyceae (golden algae), and (iii) the prokaryotic phylum Cyanobacteria (blue-green algae). The term “microalgae” includes for example genera selected from: Achnanthes, Amphora, Anabaena, Anikstrodesmis, Arachnoidiscusm, Aster, Botryococcus, Chaetoceros, Chlamydomonas, Chlorella, Chlorococcum, Chorethron, Cocconeis, Coscinodiscus, Crypthecodinium, Cyclotella, Cylindrotheca, Desmodesmus, Dunaliella, Emiliana, Euglena, Fistulifera, Fragilariopsis, Gyrosigma, Hematococcus, Isochrysis, Lampriscus, Monochrysis, Monoraphidium, Nannochloris, Nannochloropsis, Navicula, Neochloris, Nephrochloris, Nephroselmis, Nitzschia, Nodularia, Nostoc, Odontella, Oochromonas, Oocystis, Oscillartoria, Pavlova, Phaeodactylum, Playtmonas, Pleurochrysis, Porhyra, Pseudoanabaena, Pyramimonas, Scenedesmus, Schyzochitrium, Stichococcus, Synechococcus, Synechocystis, Tetraselmis, Thalassiosira, and Trichodesmium. In particular embodiments, the microalgae belong to the eukaryotic phylum Heterokontophyta or Heterokonta, which includes, without limitation, the classes Bacillariophycea (diatoms), Eustigmatophycea, Phaeophyceae (brown algae), Xanthophyceae (yellow-green algae) and Chrysophyceae (golden algae). In more particular embodiments, the microalgae belong to the Eustigmatophycea, such as to the Nannochloropsis genus or to the Bacillariophycea, such as to the Phaeodactylum genus.

The term “transformation” means introducing an exogenous nucleic acid into an organism so that the nucleic acid is replicable, either as an extrachromosomal element or by chromosomal integration.

The terms “genetically engineered” or “genetically modified” or “recombinant” as used herein with reference to a host cell, in particular a micro-organism such as a microalga, denote a non-naturally occurring host cell, as well as its recombinant progeny, that has at least one genetic alteration not found in a naturally occurring strain of the referenced species, including wild-type strains of the referenced species. Such genetic modification is typically achieved by technical means (i.e. non-naturally) through human intervention and may include, e.g., the introduction of an exogenous nucleic acid and/or the modification, over-expression, or deletion of an endogenous nucleic acid.

The term “exogenous” or “foreign” as used herein is intended to mean that the referenced molecule, in particular nucleic acid, is not naturally present in the host cell.

The term “endogenous” or “native” as used herein denotes that the referenced molecule, in particular nucleic acid, is present in the host cell.

By “recombinant nucleic acid” when referring to a nucleic acid in a recombinant host cell, in particular a recombinant micro-organism such as a recombinant microalga, is meant that at least part of said nucleic acid is not naturally present in the host cell in the same genomic location. For instance a recombinant nucleic acid can comprise a coding sequence naturally occurring in the host cell under control of an exogenous promotor, or it can be an additional copy of a gene naturally occurring in the host cell, or a recombinant nucleic acid can comprise an exogenous coding sequence under the control of an endogenous promoter.

By “nucleic acid” is meant oligomers and polymers of any length composed essentially of nucleotides, e.g., deoxyribonucleotides and/or ribonucleotides. Nucleic acids can comprise purine and/or pyrimidine bases and/or other natural (e.g., xanthine, inosine, hypoxanthine), chemically or biochemically modified (e.g., methylated), non-natural, or derivatised nucleotide bases. The backbone of nucleic acids can comprise sugars and phosphate groups, as can typically be found in RNA or DNA, and/or one or more modified or substituted sugars and/or one or more modified or substituted phosphate groups. Modifications of phosphate groups or sugars may be introduced to improve stability, resistance to enzymatic degradation, or some other useful property. A “nucleic acid” can be for example double-stranded, partly double stranded, or single-stranded. Where single-stranded, the nucleic acid can be the sense strand or the antisense strand. The “nucleic acid” can be circular or linear. The term “nucleic acid” as used herein preferably encompasses DNA and RNA, specifically including genomic, hnRNA, pre-mRNA, mRNA, cDNA, recombinant or synthetic nucleic acids, including vectors.

The term “gene” when referring to protein X, refers to the nucleic acid sequence in a given organism encoding said protein.

By “encoding” is meant that a nucleic acid sequence or part(s) thereof corresponds, by virtue of the genetic code of an organism in question, to a particular amino acid sequence, e.g., the amino acid sequence of a desired polypeptide or protein. By means of example, nucleic acids “encoding” a particular polypeptide or protein, e.g. an enzyme, may encompass genomic, hnRNA, pre-mRNA, mRNA, cDNA, recombinant or synthetic nucleic acids.

Preferably, a nucleic acid encoding a particular polypeptide or protein may comprise an open reading frame (ORF) encoding said polypeptide or protein. An “open reading frame” or “ORF” refers to a succession of coding nucleotide triplets (codons) starting with a translation initiation codon and closing with a translation termination codon known per se, and not containing any internal in-frame translation termination codon, and potentially capable of encoding a polypeptide or protein. Hence, the term may be synonymous with “coding sequence” as used in the art.

The terms “polypeptide” and “protein” are used interchangeably herein and generally refer to a polymer of amino acid residues linked by peptide bonds, and are not limited to a minimum length of the product. Thus, peptides, oligopeptides, polypeptides, dimers (hetero- and homo-), multimers (hetero- and homo-), and the like, are included within the definition. Both full-length proteins and fragments thereof are encompassed by the definition. The terms also include post-expression modifications of the polypeptide, for example, glycosylation, acetylation, phosphorylation, etc. Furthermore, for purposes of the present invention, the terms also refer to such when including modifications, such as deletions, additions and substitutions (e.g., conservative in nature), to the sequence of a native protein or polypeptide.

The term “variant”, when used in connection to a protein, such as an enzyme, for example as in “a variant of protein X”, refers to a protein, such as an enzyme, that is altered in its sequence compared to protein X, but that retains the activity of protein X, such as the enzymatic activity (i.e. a functional variant or homolog).

The term “mutant”, as used herein refers to a protein comprising in its amino acid sequence additions, deletions and/or substitutions introduced via targeted mutagenesis of the respective nucleic acid sequence. In particular embodiments, the mutant retains at least part of its activities. Accordingly, in particular embodiments, a mutant of a wild-type acyl-CoA synthase enzyme is capable of ensuring at least 60%, preferably at least 70% or 80%, of the activity of that wild-type acyl-CoA synthase enzyme. Suitable assays to determine the acyl-CoA synthase activity are known to the skilled person and involve, for instance, the use of radiolabeled fatty acids (as described for instance in Joyard J. et al. Plant Physiol. 1981 February; 67(2):250-6), or fluoro or colorimetric assays (such as described by Kuang et al. 2007, J Biochem Biophys Methods 70(4):649-655; Lageweg et al. 1991, Anal Biochem 197(2):384-388).

In particular embodiments, the mutant has at least 80%, more preferably at least 85%, even more preferably at least 90%, and yet more preferably at least 95% such as at least 96%, at least 97%, at least 98% or at least 99% sequence identity to the reference protein, preferably calculated over the entire length of the sequence. It is understood that the mutant proteins or enzymes described herein may have either or both non-conservative or essential amino acid substitutions, which do have a substantial effect on protein function and conservative or non-essential amino acid substitutions, which do not have a substantial effect on the protein function. Whether or not a particular substitution will be tolerated (i.e., will not adversely affect desired biological properties) can be determined as described in Bowie et al. (1990) (Science 247:1306 1310).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

The term “homolog” as used herein in connection to a protein, such as an enzyme, for example as in “a homolog of protein X” refers to the fact that the protein differs from protein X in its sequence, but that retains the activity or protein X, such as the enzymatic activity as detailed above, and originates from another species, i.e. is a naturally occurring sequence. A homolog of protein X can be identified by the skilled person by pairwise search methods such as BLAST and checking of the corresponding activity.

As used herein, the terms “identity” and “identical” and the like are used interchangeably with the terms “homology” and “homologues” and the like herein and refer to the sequence similarity between two polymeric molecules, e.g., between two nucleic acid molecules or polypeptides. Methods for comparing sequences and determining sequence identity are well known in the art. By means of example, percentage of sequence identity refers to a percentage of identical nucleic acids or amino acids between two sequences after alignment of these sequences. Alignments and percentages of identity can be performed and calculated with various different programs and algorithms known in the art. Preferred alignment algorithms include BLAST (available for instance at the NCBI website) and Clustal (available for instance at the EBI website). Preferably, BLAST is used to calculate the percentage of identity between two sequences, such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3).

As used herein, the term “molecules of interest” refers to any molecule which can be produced by micro-organisms, including but not limited to molecules of the lipid metabolic pathway or derived from the acetyl-CoA pool in a micro-organism. Such molecules of interest include, without limitation, hydrocarbons, fatty acids and lipids. Such molecules of interest can be recovered from the micro-organism or its culture medium, and then used in certain applications.

As used herein, the term “lipid metabolic pathway” refers to any pathway in a micro-organism comprised between acetyl-CoA and lipids (it being understood that acetyl-CoA is included in such lipid metabolic pathway). Said term hence encompasses without limitation fatty acid synthesis pathways, pathways ensuring the assembly of triacylglycerols (TAGs) or the conversion of any lipids to TAGs, and pathways degrading TAGs (beta-oxidation).

As used herein, “triacylglycerols”, also referred to as “triacylglycerides” or “TAG” are esters resulting from the esterification of the three hydroxyl groups of glycerol, with three fatty acids.

The present application generally relates to production of molecules of interest, in particular production of molecules of the lipid metabolic pathway, including production of triacylglycerol (TAG) and any intermediates in the lipid metabolic pathway, in micro-organisms, in particular in microalgae. The application is further directed to the production of biomolecules derived from said molecules of the lipid metabolic pathway.

More particularly, the application provides methods for increasing TAG production in micro-organisms, in particular microalgae, by genetically engineering the micro-organisms, in particular the microalgae, to modify the expression of acyl-CoA synthase. More particularly, the micro-organism is modified to express or overexpress an artificial variant or mutant acyl-CoA synthase (herein also referred to as “ACS”) isoform, in particular an ACS isoform belonging to the ACS Bubblegum subfamily (herein also referred to as “ACSBG”). The application also encompasses the recombinant micro-organisms as well as their use, e.g. for fatty acid production.

It has been surprisingly found that the flux of fatty acids towards triacylglycerol can be increased in microalgae by mutation of a gene encoding an acyl-CoA synthase (ACS), particularly encoding an isoform of an ACS belonging to the Bubblegum type (ACSBG). ACS are involved in the activation of fatty acids, forming acyl-CoA precursors, which are specifically used for different glycerolipid molecules. The present inventors have found that by genetic engineering of microalgae, more particularly by mutating at least one ACSBG isoform or by introducing the mutated ACSBG isoform into the microalgae, fatty acids were diverted toward an increased production of triacylglycerol. In particular, the inventors have developed a mutant Nannochloropsis gaditana ACS, particularly a “bubblegum type” ACS, which is an enzyme acting in the activation (or maturation) of fatty acids in the microalga Nannochloropsis gaditana. The inventors have found that the microalgae expressing the mutated ACSBG exhibited a decrease of some fatty acids in membrane lipids and an increase in TAG content.

Accordingly, in a first aspect, the application provides a method for increasing the production of molecules of interest in a micro-organism, in particular a microalga, said method comprising culturing said recombinant micro-organism, in particular a recombinant microalga, which has been genetically engineered to express a mutant acyl-CoA synthase (ACS), particularly a mutant acyl-CoA synthase belonging to the ACS Bubblegum or lipidosin subfamily (ACSBG). As a result of the genetic engineering, the production of said molecule of interest by said recombinant micro-organism is enhanced compared to the wild type micro-organism from which the recombinant micro-organism is derived. In particular embodiments, the invention thus relates to a method for the production of (a) molecule(s) of interest, which encompasses the steps of (i) genetically engineering a micro-organism, in particular a microalga, to express a mutant or an artificial variant of an ACS, particularly ACSBG; and (ii) culturing the recombinant micro-organism, in particular the recombinant microalga obtained in step (i), so as to allow the production of said molecule(s) of interest.

In particular embodiments, the molecule(s) of interest is/are molecule(s) of the lipid metabolic pathway or biomolecules derived from said molecules and the production of such molecule(s) of interest is increased according to the invention. In further particular embodiments, the molecule(s) of interest is/are lipids, in particular triacylglycerols (TAGs).

In particular embodiments, the methods also comprise the recovery of said molecule(s) of interest.

In certain embodiments, the methods encompass introducing one or more mutations into an ACS gene, particularly into an ACSBG gene of said micro-organism, and culturing the mutated micro-organism so obtained under suitable conditions so as to allow production of the desired molecule(s) or biomolecule(s) by the micro-organism.

In further embodiments, the methods encompass transforming a micro-organism, particularly a microalga, with a recombinant nucleic acid encoding a mutant ACS, particularly a mutant ACSBG, and culturing the recombinant micro-organism so obtained under suitable conditions so as to allow production of the desired molecule(s) or biomolecule(s) of interest by the micro-organism. Preferably in this embodiment, the recombinant organisms no longer contain an endogenous (unmutated) ACSBG gene. Preferably, the conditions suitable to allow production of the desired molecule(s) or biomolecule(s) of interest by the micro-organism are non-starved conditions.

The invention particularly relates to methods which involve introducing mutations in genes encoding Acyl CoA synthases (ACS), more particularly mutations in genes encoding ACSBG, micro-organisms resulting from said methods and uses of said micro-organisms. ACS enzymes are involved in the activation of free fatty acids into acyl-CoA prior to their specific incorporation into a variety of molecules by the action of acyl-CoA-dependent acyltransferases. The present inventors have surprisingly found that mutated ACSBG enzymes are of particular interest for increasing TAG accumulation in microalgae. In particular embodiments, the methods of the invention comprise introducing a mutant nucleic acid sequence encoding ACSBG into a Heterokonta microalga. In further embodiments, the microalga belongs to the Eustigmatophycea or Eustigmatophytes or the Bacillariophycea. In yet further particular embodiments, the microalga is of the Nannochloropsis genus or of the Phaeodactylum genus. In yet further particular embodiments, the microalga is N. gaditana or P. tricornutum. In particular embodiments, the nucleic acid sequence encoding said ACSBG is a gene encoding ACSBG from a Heterokonta microalga, more particularly from a Eustigmatophycea, Eustigmatophytes or Bacillariophycea, More particularly from N. gaditana or P. tricornutum.

Alternatively, the methods may comprise introducing one or more mutations into an endogenous ACS gene, particularly ACSBG gene, as envisaged herein, in a micro-alga, more particularly, a Heterokonta microalga. In particular embodiments, the microalga belongs to the Eustigmatophycea or Eustigmatophytes or the Bacillariophycea. In yet further particular embodiments, the microalga is of the Nannochloropsis genus or of the Phaeodactylum genus. In yet further particular embodiments, the microalga is N. gaditana or P. tricornutum.

Accordingly, the micro-organisms obtained according to the methods of the invention comprise a recombinant nucleic acid or mutated gene which encodes a mutant of an ACSBG, preferably a mutant of an ACSBG of microbial origin, particularly a mutant of an ACSBG from a microalga, more particularly a Heterokonta microalga, even more particularly a Eustigmatophycea or Eustigmatophytes or Bacillariophycea microalga, such as a Nannochloropsis or Phaeodactylum microalga. In particular embodiments, the mutant ACSBG is functional, i.e. the mutant ACSBG retains at least part of the wild-type ACSBG activity. More particularly, a mutant of an acyl-CoA synthase enzyme, such as a mutant ACSBG, is capable of ensuring at least 60%, preferably at least 70% or 80%, of the activity of that particular acyl-CoA synthase enzyme. Suitable assays to determine the acyl-CoA synthase activity are known to the skilled person and involve, for instance, the use of radiolabeled fatty acids (Joyard J. and Stumpf P K. 1981, Plant Physiol. 67(2):250-6, fluorometric assays (Lageweg et al. 1991, Anal. Biochem. 197(2): 384-388) or colorimetric assays (Kuang et al., 2007) J Biochem Biophys Methods. 70(4): 649-655). In particular embodiments, the mutant acyl-CoA synthase enzyme, in particular the mutant ACSBG, maintains detectable ACSBG activity.

In particular embodiments, the methods encompass introducing a mutation in an ACSBG gene, i.e. a gene corresponding to the ACSBG gene which encodes a protein having an amino acid sequence corresponding to the amino acid sequence of ACSBG protein of N. gaditana or P. tricornutum, also referred to herein as NgACSBG or PtACSBG, respectively, or a variant or a homolog thereof. As used herein the term “NgACSBG” refers to a protein having an amino acid sequence SEQ ID NO. 7. As used herein the term “PtACSBG” refers to a protein having an amino acid sequence SEQ ID NO. 8. In further particular embodiments, the methods encompass introducing a mutation in an ACSBG gene encoding a protein having SEQ ID NO. 7 or SEQ ID NO. 8. Alternatively, the methods may comprise introducing a polynucleotide encoding for a mutant of an ACSBG protein having SEQ ID NO. 7 or SEQ ID NO. 8, or a variant or homolog thereof, into a micro-alga as considered herein.

In particular embodiments, the methods encompass introducing a mutation in an ACSBG gene encoding a protein corresponding to a homolog of NgACSBG or PtACSBG. In particular embodiments, said homolog originates from a microalga. Preferably, a NgACSBG homolog has a sequence substantially identical to SEQ ID NO.7, or a sequence having at least about 70%, preferably at least about 80%, more preferably at least about 85%, 90% or 95%, even more preferably at least about 96%, 97%, 98% or 99% sequence identity to SEQ ID NO.7. Preferably, a PtACSBG homolog has a sequence substantially identical to SEQ ID NO.8, or a sequence having at least about 70%, preferably at least about 80%, more preferably at least about 85%, 90% or 95%, even more preferably at least about 96%, 97%, 98% or 99% sequence identity to SEQ ID NO.8.

It will be understood by the skilled person that, in addition to the sequence similarity as defined herein, the NgACSBG or PtACSBG homologs or variants, including the mutant NgACSBG or PtACSBG homologs or variants as envisaged herein, are further defined by the specific conservation of Motif II of the “bubblegum”-type of ACS, as described by (Steinberg et al., 2000, J. Biol Chem 275: 35162-35169), wherein the conserved motif II has the sequence GXXXXTGRXKELIITAGGENXPPVXIEXXXKXXXP (SEQ ID NO. 1), wherein X is any amino acid residue.

For instance, the present inventors have identified ACSBG sequences, homologous to NgACSBG or PtACSBG, in other Nannochloropsis sp., in particular N. occulata and N. oceanica—with amino acid sequence SEQ ID NO. 9 and SEQ ID NO. 10, respectively. Also, the present inventors have identified ACSBG sequences, homologous to NgACSBG or PtACSBG, in the genera Pithyum, such as an ACSBG with amino acid sequence SEQ ID NO. 11; Phytophtora, such as an ACSBG with amino acid sequence SEQ ID NO. 12; Ectocarpus, such as an ACSBG with amino acid sequence SEQ ID NO. 13; and Fistulifera, such as an ACSBG with amino acid sequence SEQ ID NO. 14. In certain other particular embodiments, the methods according to the present invention encompass introducing a mutation in an ACSBG gene encoding a protein having SEQ ID NO. 9, SED ID NO. 10, SEQ ID NO. 11, SEQ ID NO. 12, SEQ ID NO. 13 or SEQ ID NO. 14, or an ACSBG gene encoding a protein having one of SEQ ID NO:22-28.

The inventors have found that the introduction of a mutated ACSBG protein in a micro-organism can result in an increased TAG production. The mutations are preferably in a region of the ACSBG protein located between amino acids 72 and 120 of SEQ ID NO:7, more particularly in an extended/coil region spanning from amino acid 83 to 102 between two alpha helices in SEQ ID NO:7, which is NgACSBG or in the corresponding region in an ACSBG from another microalga species. More particularly, the mutation is in SEQ ID NO: 17 of NgACSBG or a corresponding region in an ACSBG from another microalga species. In particular embodiments, the one or more mutations, in particular one or more amino acid substitutions and/or additions, are in the region between amino acids 96 and 98 of SEQ ID NO: 7, also referred to herein as IGF triad or IGF domain, or in the corresponding region in an ACSBG from another microalga species.

In particular embodiments, the one or more mutations in the sequence encoding the ACSBG protein correspond to mutations resulting in 1-5 amino acid substitutions, deletions or additions, particularly 1-3 amino acid substitutions, deletions or additions, in the region corresponding to the region between amino acids 83 and 102 of amino acid sequence SEQ ID NO. 7. Indeed it has been established that mutations in this region of the ACSBG protein when expressed in a micro-organism result an increase in TAG production in the micro-organism, resulting in an increase in TAG and/or downstream products of TAG. More particularly, the one or more mutations are between amino acids 83 and 102 of amino acid sequence SEQ ID NO. 7, corresponding to the amino acid sequence of SEQ ID NO. 17, or in a corresponding region of an ACSBG of another microalga species. In particular embodiments, the mutation is at one or more of positions 96-98 of SEQ ID NO:7 or corresponding amino acids in an ACSBG of another microalga species. Indeed, the one or more mutations described above for SEQ ID NO:7 can alternatively be in the corresponding region of a homolog of the NgACSBG having amino acid sequence SEQ ID NO. 7, which can be identified by aligning the homologous sequence with amino acid SEQ ID NO. 7. As described above, sequences can be aligned with various different programs and algorithms known in the art, including BLAST and Clustal W. In more particular embodiments, the mutation introduced into an ACSBG gene as envisaged herein, yields a mutant ACSBG gene, which encodes for a mutant ACSBG, having 1-5 amino acid substitutions, deletions or additions, particularly 1-3 amino acid substitutions, deletions or additions, in the region between amino acid 72 and 120, more particularly between amino acid 83 and 102 of amino acid sequence SEQ ID NO: 7, most particularly at positions 96-98 of SEQ ID NO: 7 or in the corresponding region or positions of a homolog. In particular embodiments the one or more mutations in the ACSBG gene result in one or more of the following in the region corresponding to SEQ ID NO: 17 in the ACSBG protein: (a) the addition of one or more amino acids, such as an Arginine or a Glutamine, in particular a Glutamine, in said region; (b) the change of one or more Isoleucines or Glycines to another amino acid, such as Phenylalanine or Arginine or Valine, in particular the change of one or more Isoleucines to a Phenylalanine or an Arginine and/or the change of one or more Glycines to a Phenylalanine, an Arginine or ar a Valine and/or (c) the change of one or more Phenylalanine to another amino acid such as Leucine or Glutamine, in particular Leucine. In further particular embodiments the one or more mutations in the ACSBG gene result in one or more of the following: (a) an additional amino acid, more particularly an Arginine introduced at a position corresponding to between amino acid 95 and 96 in SEQ ID NO. 7 or a Glutamine introduced at a position corresponding to between amino acid 98 and 99 in SEQ ID NO. 7, in particular a Glutamine introduced at a position corresponding to between amino acid 98 and 99 in SEQ ID NO. 7; (b) a change of an Isoleucine in a position corresponding to AA96 to another amino acid, such as Phenylalanine or Arginine; (c) a change of a Glycine in a position corresponding to AA97 to another amino acid such as Arginine or Phenylalanine or Valine; (d) a change of Phenylalanine in a position corresponding to AA98 in SEQ ID NO. 7 to another amino acid, such as Leucine or Glutamine, in particular Leucine. In certain embodiments, the mutations result in all modifications (a) to (d), in other embodiments, the modifications result in at least one, or at least two or at least three of said modifications. In certain embodiments, the mutant ACSBG protein has at least one amino acid change with respect to SEQ ID NO. 7 and has an amino acid sequence having at least about 70%, preferably at least about 80%, more preferably at least about 85%, 90% or 95%, even more preferably at least about 96%, 97%, 98% or 99% sequence identity to SEQ ID NO.15, SEQ ID NO. 16 or SEQ ID NO. 44. In particular embodiments, the mutant ACSBG protein comprises the corresponding mutations of SEQ ID NO. 15, SEQ ID NO. 16 or SEQ ID NO. 44 with respect to SEQ ID NO. 7.

In more particular embodiments, the mutation introduced into an ACSBG gene as envisaged herein, yields a mutant ACSBG gene, which encodes for a mutant ACSBG protein having the amino acid sequence SEQ ID NO. 15, SEQ ID NO. 16 or SEQ ID NO.44.

In certain embodiments, the methods encompass introducing a mutation in a PtACSBG gene, wherein the mutation introduced into the PtACSBG gene yields a mutant ACSBG gene, encoding a mutant PtACSBG having 1-5, such as 1-3, amino acid substitutions, deletions or additions in the region between amino acid 74 and 122, such as between amino acid 85 and 104, of amino acid sequence SEQ ID NO. 8, more particularly between amino acids 98 and 100 of amino acid sequence SEQ ID NO. 8.

In certain embodiments, the methods encompass introducing a mutation in an ACSBG gene from a Nannochloropsis sp., such as N. occulata or N. oceanica and wherein the mutation introduced in the ACSBG gene yields a mutant ACSBG gene encoding a mutant ACSBG, having 1-5, such as 1-3, amino acid substitutions, deletions or additions in the region between amino acid 73 and 123, more particularly, between amino acid 83 and 102, such as between amino acid 96 and 98 of amino acid sequence SEQ ID NO. 9 or SEQ ID NO. 10.

In certain embodiments, the methods encompass introducing a mutation in an ACSBG gene from a Pythium sp., such as Pythium insidiosum, and wherein the mutation introduced in the ACSBG gene yields a mutant ACSBG gene encoding a mutant ACSBG, having 1-5, such as 1-3, amino acid substitutions, deletions or additions in the region between amino acid 76 and 94 of amino acid sequence SEQ ID NO. 11, particularly such as having 1-5, such as 1-3, amino acid substitutions, deletions or additions in the region between amino acid 76 and 94, more particularly and one or more of positions 88-90 of amino acid sequence SEQ ID NO. 11.

In certain embodiments, the methods encompass introducing a mutation in an ACSBG gene from a Phytophtora sp., such as Phytophtora parasitica, wherein the mutation introduced in the ACSBG gene yields a mutant ACSBG gene encoding a mutant ACSBG, having 1-5, such as 1-3, amino acid substitutions, deletions or additions in the region between amino acid 76 and 95 of amino acid sequence SEQ ID NO. 12, more particularly at one or more of positions 98-91 of amino acid sequence SEQ ID NO. 12.

In certain embodiments, the methods encompass introducing a mutation in an ACSBG gene from a Ectocarpus sp., such as Ectocarpus siliculosus, and wherein the mutation introduced in the ACSBG gene yields a mutant ACSBG gene encoding a mutant ACSBG, having 1-5, such as 1-3, amino acid substitutions, deletions or additions in the region between amino acid 84 and 103 of amino acid sequence SEQ ID NO. 13, more particularly at one or more of positions 97-99 of amino acid sequence SEQ ID NO. 13.

In certain embodiments, the methods encompass introducing a mutation in an ACSBG gene from a Fistulifera sp., such as Fistulifera solaris, and wherein the mutation introduced in the ACSBG gene yields a mutant ACSBG gene encoding a mutant ACSBG, having 1-5 amino acid substitutions, deletions or additions in the region between amino acid 84 and 103, more particularly at one or more of positions 97-99 of amino acid sequence SEQ ID NO. 14.

In certain embodiments, the methods encompass introducing a mutation in an ACSBG G2 gene from Gallus gallus, and wherein the mutation introduced in the ACSBG gene yields a mutant ACSBG gene encoding a mutant ACSBG, having 1-5 amino acid substitutions, deletions or additions in the region between amino acid 152 and 171, more particularly at one or more of positions 165-167 of amino acid sequence SEQ ID NO. 22.

In certain embodiments, the methods encompass introducing a mutation in an ACSBG G1 gene from Gallus gallus, and wherein the mutation introduced in the ACSBG gene yields a mutant ACSBG gene encoding a mutant ACSBG, having 1-5 amino acid substitutions, deletions or additions in the region between amino acid 116 and 135, more particularly at one or more of positions 129-131 of amino acid sequence SEQ ID NO. 23.

In certain embodiments, the methods encompass introducing a mutation in an ACSBG G2 gene from Homo sapiens, and wherein the mutation introduced in the ACSBG gene yields a mutant ACSBG gene encoding a mutant ACSBG, having 1-5 amino acid substitutions, deletions or additions in the region between amino acid 98 and 117, more particularly at one or more of positions 111-113 of amino acid sequence SEQ ID NO. 24.

In certain embodiments, the methods encompass introducing a mutation in an ACSBG G1 gene from Homo sapiens, and wherein the mutation introduced in the ACSBG gene yields a mutant ACSBG gene encoding a mutant ACSBG, having 1-5 amino acid substitutions, deletions or additions in the region between amino acid 150 and 169, more particularly at one or more of positions 163-165 of amino acid sequence SEQ ID NO. 25.

In certain embodiments, the methods encompass introducing a mutation in an ACSBG G1 gene from Mus musculus, and wherein the mutation introduced in the ACSBG gene yields a mutant ACSBG gene encoding a mutant ACSBG, having 1-5 amino acid substitutions, deletions or additions in the region between amino acid 147 and 166, more particularly at one or more of positions 160-162 of amino acid sequence SEQ ID NO. 26.

In certain embodiments, the methods encompass introducing a mutation in an ACSBGa gene from Drosophila melanogaster, and wherein the mutation introduced in the ACSBG gene yields a mutant ACSBG gene encoding a mutant ACSBG, having 1-5 amino acid substitutions, deletions or additions in the region between amino acid 94 and 113, more particularly at one or more of positions 107-109 of amino acid sequence SEQ ID NO. 27.

In certain embodiments, the methods encompass introducing a mutation in an ACSBGc gene from Drosophila melanogaster, and wherein the mutation introduced in the ACSBG gene yields a mutant ACSBG gene encoding a mutant ACSBG, having 1-5 amino acid substitutions, deletions or additions in the region between amino acid 94 and 113, more particularly at one or more of positions 107-109 of amino acid sequence SEQ ID NO. 28.

In certain embodiments, the methods may comprise introducing, into a microalga as considered herein, a mutated polynucleotide encoding for one or more of said mutants of an ACSBG protein having SEQ ID NO. 9, SED ID NO. 10, SEQ ID NO. 11, SEQ ID NO. 12, SEQ ID NO. 13, SEQ ID NO. 14 or of SEQ ID NO:22-28 as described above and optionally inactivating the endogenous ACSBG protein. In particular embodiments the method may comprise introducing into a microalga as considered herein, a mutated polynucleotide encoding an ACSBG protein having SEQ ID: 15, 16 or 44 into said micro-organism.

The methods provided herein envisage genetically engineering micro-organisms, in particular microalgae, for producing an increased amount of certain molecules of interest. Accordingly, the herein described methods aim to increase the production of molecules of interest, in particular molecules of the lipid metabolic pathway, including lipids as well as intermediates of said lipid metabolic pathway such as fatty acids. More particularly, the methods relate to increasing the production of molecules, the synthesis of molecules which are produced in a TAG-dependent pathway. In particular embodiment, the fatty acid is one or more of C1-C10, C12:0, C14:0, C16:0, C16:1, C18:0, C18:1, C18:2, C18:3, C20-C24 or a derivative thereof. In addition, the herein described methods increase the production of biomolecules derived from said molecules of the lipid metabolic pathway such as hydrocarbons, fatty alcohols, etc. In particular embodiments of the method, the production of lipids, more particularly TAGs, is increased.

In particular embodiments, the methods provided herein envisage transforming micro-organisms, in particular microalgae, to stimulate the lipid metabolic pathway and/or to increase production of biomolecules derived from molecules of the lipid metabolic pathway. Accordingly, in particular embodiments, the methods encompass providing a microbial strain suitable for lipid production or wherein the lipid production is to be increased. Such a strain is preferably a microbial strain which produces lipids. In preferred embodiments, the micro-organism is a microalga. Preferably, the microalga is selected from the Chromalveolata, more preferably the Heterokontophyta, even more preferably the Bacillariophyceae (diatoms), including the Naviculales such as Phaeodactylum tricornutum and the Pennales (pennate diatoms), and/or the Eustigmatophyceae, including Nannochloropsis species. In particular embodiments, the microalga is Nannochloropsis gaditana. In other particular embodiments, the microalga is Phaeodactylum tricornutum.

In particular embodiments, the methods include optimizing the recombinant micro-organisms as taught herein and/or the cultivation medium so as to ensure the production of the biomolecule of interest. This can encompass further modifying the mutant micro-organism so as to further the production and/or preventing the catabolism of the biomolecule, for instance by blocking other biosynthesis pathways. For instance, where production of TAG is envisaged, the methods of the present invention may additionally comprise modifying the micro-organism of interest by blocking of pathways that divert carbon to alternative metabolic routes and/or preventing TAG catabolism. Such methods have been described in the art, as noted in the background section herein. Additionally or alternatively the cultivation or production conditions can be adjusted to stimulate the production of the biomolecule of interest. In particular embodiments, the micro-organisms are modified to block non-desirable pathways, such as programmed cell death. In particular embodiments, the methods of the invention comprise maintaining the cell under conditions which ensure that cell viability is maintained or is at an acceptable level.

In particular embodiments, the recombinant or mutant micro-organisms, preferably microalgae, described herein are further genetically modified to ensure production of molecules of interest, in particular molecules of the lipid metabolic pathway or biomolecules derived from said molecules. Further genetic modifications to further increase lipid production, in particular TAG production, as well as the production of any intermediate of the lipid metabolic pathway, are envisaged herein, but also further genetic modifications to ensure (increased) production of a biomolecule of interest derived from a molecule of the lipid metabolic pathway. For example, the recombinant micro-organisms described herein may be further genetically modified to further increase fatty acid biosynthesis and/or TAG assembly. In other examples, the recombinant micro-organisms may be further genetically modified to ensure production of e.g. hydrocarbons from fatty acids, or fatty alcohols from acetyl-CoA. Also, methods for increasing expression of an endogenous nucleic acid are known in the art and include but are not limited to introducing one or more copies of the endogenously present nucleic acid, optionally under control of stronger promoters, introducing transcription activators, capable of activating transcription of the endogenous gene etc.

As metabolic pathways are well-established in micro-organisms, methods for modifying the lipid metabolic pathway and the production of biomolecules derived from molecules of said lipid metabolic pathway in a micro-organism as described herein can be easily determined by the skilled person, including in microalgae. Standard reference work setting forth the general principles of biochemistry includes “Biochemical Pathways: An Atlas of Biochemistry and Molecular Biology”, ed. Michal, G, John Wiley and Sons, Inc., New York, US, 1999. In the field of microalgae, standard reference work includes: “Handbook of Microalgal Culture: Applied Phycology and Biotechnology”, 2nd Edition, Amos Richmond and Qiang Hu, WileyBlackwell, 2013.

Most microalgae are photoautotrophs, i.e. their growth is strictly dependent on the generation of photosynthetically-derived energy. Their cultivation hence requires a relatively controlled environment with a large input of light energy. For certain industrial applications, it is advantageous to use heterotrophic microalgae, which can be grown in conventional fermenters. In particular embodiments the microalgae have been further metabolically engineered to grow heterotrophically (i.e. to utilize exogenous organic compounds (such as glucose, acetate, etc.) as an energy or carbon source). A method for metabolically engineering microalgae to grow heterotrophically has been described in U.S. Pat. No. 7,939,710, which is specifically incorporated by reference herein. In particular embodiments, the microalgae are further genetically engineered to comprise a recombinant nucleic acid encoding a glucose transporter, preferably a glucose transporter selected from the group consisting of Glut 1 (human erythrocyte glucose transporter 1) and Hup1 (Chlorella HUP1 Monosaccharide-H+ Symporter). The glucose transporters facilitate the uptake of glucose by the host cell, allowing the cells to metabolize exogenous organic carbon and to grow independent of light. This is particularly advantageous for obligate phototrophic microalgae. Lists of phototrophs may be found in a review by Droop (1974. Heterotrophy of Carbon. In Algal Physiology and Biochemistry, Botanical Monographs, 10:530-559, ed. Stewart, University of California Press, Berkeley), and include, for example but without limitation, organisms of the phyla Cyanophyta (Blue-green algae), including the species spirulina and anabaena; Chlorophyta (Green algae), including the species dunaliella, chlamydomonas, and heamatococcus; Rhodophyta (Red algae), including the species porphyridium, porphyra, euchema, and graciliaria; Phaeophyta (Brown algae), including the species, macrocystis, laminaria, undaria, and fucus; Baccilariophyta (Diatoms), including the species nitzschia, navicula, thalassiosira, and phaeodactylum; Dinophyta (Dinoflagellates), including the species gonyaulax; Chrysophyta (Golden algae), including the species irsochrysis and nannochloropsis; Cryptophyla, including the species cryptomonas; and Euglenophyta, including the species euglena.

In the methods envisaged herein, the recombinant micro-organisms are preferably cultured under conditions suitable to increase production of the molecules of interest.

More particularly this implies “conditions sufficient to allow expression” of the recombinant ACSBG considered herein. Typically, the culture conditions are also selected so as to favor production of molecules of interest, in particular molecules of the lipid metabolic pathway or biomolecules derived from said molecules. In particular embodiments, the conditions are non-starved conditions, i.e. which allow maximal production of said product of interest, i.e. which do not intentionally limit the production thereof by the absence of one or more components or cultivation conditions.

Culture conditions can comprise many parameters, such as temperature ranges, levels of aeration, and media composition. Each of these conditions, individually and in combination, allows the micro-organism to grow. To determine if conditions are sufficient to allow (over)expression, a micro-organism can be cultured, for example, for about 4, 8, 12, 18, 24, 36, or 48 hours. During and/or after culturing, samples can be obtained and analyzed to determine if the conditions allow (over)expression. For example, the micro-organisms in the sample or the culture medium in which the micro-organisms were grown can be tested for the presence of a desired product, e.g. a transcript of the recombinant nucleic acid. When testing for the presence of a desired product, assays, such as, but not limited to, polymerase chain reaction (PCR), sodium dodecyl sulfate polyacrylamide gel electrophoresis (SDS-PAGE), TLC, HPLC, GC/FID, GC/MS, LC/MS, MS, can be used. In particular, when testing for the presence or expression of an ACS, ACS activity assays as known to the skilled person can be used.

Exemplary culture media include broths or gels. The micro-organisms may be grown in a culture medium comprising a carbon source to be used for growth of the micro-organisms. Exemplary carbon sources include carbohydrates, such as glucose, fructose, cellulose, or the like, that can be directly metabolized by the host cell. In addition, enzymes can be added to the culture medium to facilitate the mobilization (e.g., the depolymerization of starch or cellulose to fermentable sugars) and subsequent metabolism of the carbon source. A culture medium may optionally contain further nutrients as required by the particular strain, including inorganic nitrogen or phosphorous sources, and the like, and minerals and the like. In particular embodiments, wherein phototrophic microalgae are used, the method for increasing the production of molecules of interest, in particular molecules of the lipid metabolic pathway or biomolecules derived from said molecules, may comprise providing recombinant microalgae genetically engineered as taught herein, and culturing said microalgae in photobioreactors or an open pond system using CO₂ and sunlight as feedstock.

Other growth conditions, such as temperature, cell density, and the like are generally selected to provide an economical process. Temperatures during each of the growth phase and the production phase may range from above the freezing temperature of the medium to about 50° C.

The culturing step of the methods described herein can be conducted continuously, batch-wise, or some combination thereof.

In particular embodiments, the culturing conditions which are suitable for the expression of the mutant ACS, particularly mutant ACSBG as described herein, and for the subsequent production of the molecules of interest by the recombinant micro-organisms are also suitable for growth of the recombinant micro-organism, such that the production of the molecules of interest is concomitant with the growth of the micro-organism. In other words, according to particular embodiments, in the genetically engineered strains, the production of the molecules of interest can occur during the growth phase of the micro-organism, which can increase improve overall productivity of the molecules of interest.

In particular embodiments, the methods for the increased production of molecules of interest, further comprise the step of recovering said molecules or biomolecules from the recombinant micro-organisms and/or from the cultivation medium. Accordingly, in particular embodiments the methods comprise recovering molecules of the lipid metabolic pathway, in particular lipids, more particularly TAGs, or biomolecules derived from said molecules as envisaged herein, from the recombinant micro-organism and/or from the cultivation medium.

Methods for the recovery of said molecules or biomolecules from microalgae are known in the art and typically involve cell disruption and extraction of the molecules of interest (Guldhe et al. 2014, Fuel 128:46-52). Alternatively or in addition, the recombinant micro-organisms described herein may be further genetically engineered to ensure secretion of the molecules of interest. A further example includes a hydrothermal processing (HTL) of microalgae to produce biocrude, from which the molecules of interest can be recovered or which can be further processed to e.g. biofuel.

In a further aspect, the present invention provides a recombinant or mutant micro-organism, preferably a recombinant or mutant microalga, more preferably a Heterokonta microalga, able to produce an increased amounts of a molecule of interest, in particular TAG, and wherein said recombinant or mutant micro-organism comprise a mutated ACSBG gene or nucleic acids sequence encoding a mutated ACSBG protein as described herein above. More particularly the micro-organism expresses a mutated ACSBG gene as taught herein, particularly a mutant ACSBG nucleic acid sequence encoding an ACSBG with SEQ ID NO. 7 or SEQ ID NO. 8, or a homolog thereof, as taught herein. In further embodiments, the microalga belongs to the Eustigmatophycea or Eustigmatophytes or the Bacillariophycea. In yet further particular embodiments, the microalga is of the Nannochloropsis genus or of the Phaeodactylum genus. In yet further particular embodiments, the microalga is N. gaditana or P. tricornutum, expressing a mutated NgACSBG or PtACSBG, respectively.

In certain embodiments, the recombinant micro-organisms, particularly recombinant microalgae, described herein, may comprise a mutant endogenous ACSBG gene encoding a mutant ACSBG isoform, obtained by introducing a mutation in the endogenous ACSBG. In other embodiments, the recombinant micro-organisms, particularly recombinant microalgae, described herein, may comprise an exogenous nucleic acid encoding a mutant ACSBG enzyme. Preferably in the letter embodiments, the recombinant organisms no longer contain an endogenous ACSBG gene.

As noted above, the present inventors have surprisingly found that culturing the recombinant micro-organisms described herein results in increased production of molecules of interest, in particular increased production of molecules of the lipid metabolic pathway, in particular increased lipid production, more particularly increased TAG production. Accordingly, the recombinant micro-organisms, in particular the recombinant microalgae, described herein, are characterized in that they are genetically engineered to (over)express a mutant ACS, particularly a mutant ACSBG as described herein and may further be characterized by their increased lipid content. In particular embodiments, the recombinant micro-organisms, in particular the recombinant microalgae described herein, ensure a rate of TAG production, which is sufficiently high to be industrially valuable. More particularly, the TAG content in said recombinant micro-organisms and microalgae may be to at least 110%, preferably at least 120%, more preferably at least 150% 160%, 170%, 180% or 190%, even more preferably at least 200% or more as compared to micro-organisms and microalgae which do not comprise or express the mutant ACS, particularly the mutant ACSBG as described herein, such as a wild-type micro-organisms or microalgae that have been transformed with an empty vector. In other words, the present method causes an increase in the TAG content of the recombinant micro-organisms and microalga compared to a micro-organism or microalga which do not comprise or express the mutant ACS, particularly the mutant ACSBG as described herein, by a factor of at least 1.1, preferably at least 1.2, more preferably at least 1.5, 1.6, 1.7, 1.8 or 1.9, even more preferably at least 2 or even beyond.

The lipid content, in particular the TAG content, of a micro-organism can be measured using a variety of methods well known in the art. Non-limiting examples include staining with the fluorophore Nile Red (excitation wavelength at 485 nm; emission at 525 nm) and measurement of Nile Red fluorescence, and mass spectrometry (MS).

Another aspect of the application is directed to methods for obtaining a recombinant micro-organism such as a recombinant microalga as envisaged herein, capable of expressing a mutant ACS, particularly a mutant ACSBG as described herein, so as to enhance the production of said molecule of interest compared to the wild type micro-organism from which the recombinant micro-organism is derived. In general, the methods for generating the recombinant micro-organisms described herein involve standard genetic modifications, for which well-established methods are available to the skilled person. More particularly, genetic engineering of the micro-organisms containing a recombinant nucleic acid encoding a mutant ACS, particularly a mutant ACSBG as taught herein may be accomplished in one or more steps via the design and construction of appropriate vectors and transformation of the micro-organisms with those vectors, followed by the selection of a micro-organism, particularly microalga, capable of expressing the mutant ACS, particularly mutant ACSBG as taught herein.

In particular, the mutagenesis of an endogenous ACS gene, particularly an endogenous ACSBG gene, is carried by genetic engineering. Suitable genetic engineering methods for introducing a mutation in an endogenous gene are known to the skilled person, including by using so-called molecular scissors (nucleases) (e.g. TALEN, CRISPR/Cas9 and the like), or by using vectors containing specific sequences for homologous recombination and site-directed insertion.

In addition, methods for transforming microalgae are well known to a skilled person. For example, electroporation and/or chemical (such as calcium chloride- or lithium acetate-based) transformation methods or Agrobacterium tumefaciens-mediated transformation methods as known in the art can be used.

Numerous vectors are known to practitioners skilled in the art and any such vector may be used. Selection of an appropriate vector is a matter of choice. Preferred vectors are vectors developed for microalgae such as the vectors called pH4-GUS, pCT2, and pCT2Ng.

Recombinant vectors used in the context of transformation (either for the introduction of a transgene or for the introduction of a sequence-specific nuclease) are known in the art. Typically, a vector may comprise a recombinant nucleic acid and may further contain restriction sites of various types for linearization or fragmentation. The vector may further include an origin of replication that is required for maintenance and/or replication in a specific cell type. The vector also preferably contains one or more selection marker gene cassettes. A selectable marker gene cassette typically includes a promoter and transcription terminator sequence, operatively linked to a selectable marker gene. Suitable markers may be selected from markers that confer antibiotic resistance, herbicide resistance, visual markers, or markers that complement auxotrophic deficiencies of a host cell, in particular a microalga. For example, the selection marker may confer resistance to an antibiotic such as hygromycin B (such as the hph gene), zeocin/phleomycin (such as the ble gene), kanamycin or G418 (such as the nptII or aphVIII genes), spectinomycin (such as the aadA gene), neomycin (such as the aphVIII gene), blasticidin (such as the bsd gene), nourseothricin (such as the natR gene), puromycin (such as pac gene) and paromomycin (such as the aphVIII gene). In other examples, the selection marker may confer resistance to a herbicide such as glyphosate (such as glyphosate acetyl transferas (GAT) gene), oxyfluorfen (such as protox/PPO gene) and norflurazon (such as Phytoene desaturase (PDS) gene). Visual markers may also be used and include for example beta-glucuronidase (GUS), luciferase and fluorescent proteins such as Green Fluorescent Protein (GFP), Yellow Fluorescent protein, etc. Two prominent examples of auxotrophic deficiencies are the amino acid leucine deficiency (e.g. LEU2 gene) or uracil deficiency (e.g. URA3 gene). Cells that are orotidine-5′-phosphate decarboxylase negative (ura3-) cannot grow on media lacking uracil. Thus a functional URA3 gene can be used as a selection marker on a host cell having a uracil deficiency, and successful transformants can be selected on a medium lacking uracil. Only cells transformed with the functional URA3 gene are able to synthesize uracil and grow on such medium. If the wild-type strain does not have a uracil deficiency, an auxotrophic mutant having the deficiency must be made in order to use URA3 as a selection marker for the strain. Methods for accomplishing this are well known in the art.

Successful transformants can be selected for in known manner, by taking advantage of the attributes contributed by the marker gene, or by other characteristics contributed by the inserted recombinant nucleic acid or the modified nucleic acid. Screening can also be performed by PCR or Southern analysis to confirm that the desired insertions have taken place, to confirm copy number and to identify the point of integration of coding sequences into the host genome.

Methods for modifying endogenous gene expression by the use of artificial transcription factors (ATFs) or activator domains have also been described (Sera T. 2009, Adv Drug Deliv Rev 61:513-526; Maeder et al. 2013, Nat Methods 10:243-245; Cheng et al. 2013, Cell Res 23:1163-1171).

The recombinant micro-organisms, particularly recombinant microalgae described herein may be particularly suitable for industrial applications such as biofuel production and the production of biomolecules e.g. for chemical applications, for use in food industry, for the production of cosmetics, etc. Hence, a further aspect relates to the use of the recombinant micro-organisms and microalgae described herein for biofuel production or production of biomolecules (e.g. fatty acids), e.g. for chemical industry, for food industry, for cosmetics, etc.

The present invention will now be further illustrated by means of the following non-limiting examples.

### EXAMPLES

### Example 1: Identification of an Acyl-CoA Synthase of the «Bubblegum» Subfamily (ACSBG) in Heterokonts, Including the Diatom Phaeodactylum tricornutum and the Eustigmatophyte Nannochloropsis gaditana

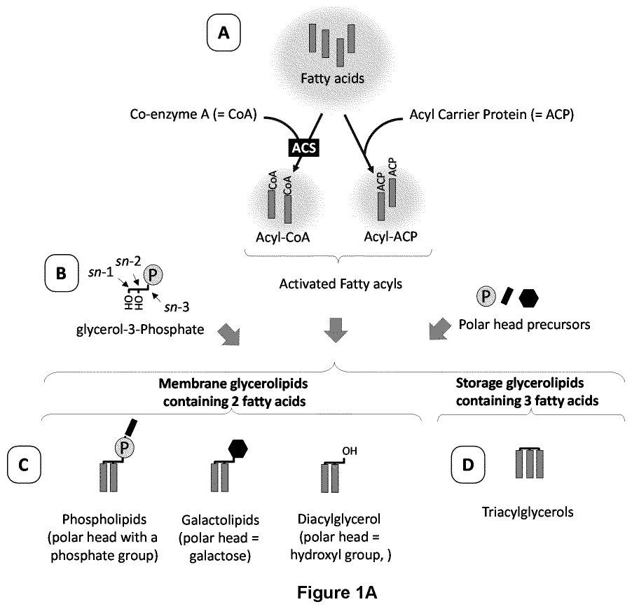

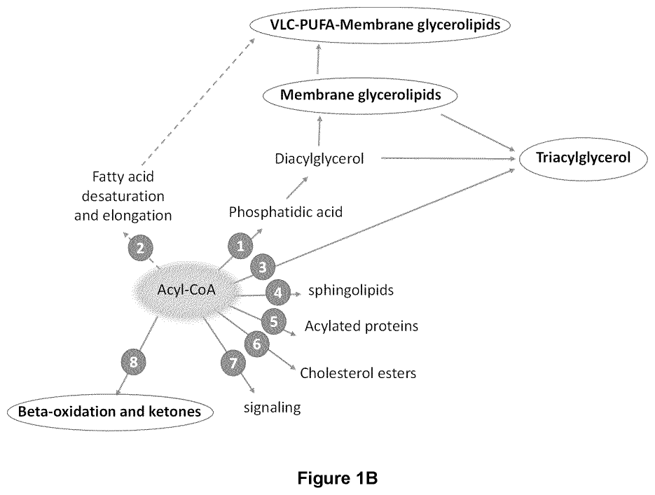

Nannochloropsis gaditana and Phaeodactylum tricornutum contain multiple genes coding for Acyl CoA synthases (ACS), which are involved in the activation of free fatty acids into acyl-CoA prior their specific incorporation into a variety of molecules by the action of acyl-CoA-dependent acyltransferases. Some Acyl-CoA are thus required to convert free fatty acids into acyl-CoA for the production of some membrane acyl-lipids, TAG, some acylated sterols, proteins, etc. Some ACS isoform can also activate free fatty acids prior their degradation inside mitochondria and peroxisome (FIG. 1B). ACS can be specific of a specific utilization of the Acyl-CoA they produce; some ACS can also play a role in more than one pathway (Coleman et al., 2002).

Subfamilies of ACS have been defined previously for eukaryotes. According to the classification of (Steinberg et al., 2000, J. Biol Chem 275: 35162-35169) one subfamily corresponds to the “bubblegum” Acyl-CoA synthases, named after the phenotype created by the mutation of the corresponding ACS gene in the fly Drosophila melanogaster, leading to a neurodegeneration and elevated level of very long-chain fatty acids. This subfamily of ACS is also called “lipidosin”.

The genome of Nannochloropsis contains 6 genes annotated as Acyl-CoA synthases (ACS) or Acyl-CoA ligases (ACL) genes in the Nannochloropsis genome internet portal (http://www.Nannochloropsis.orq): Naga_100014 g59; Naga_100012 g66; Naga_101051 g1; Naga_100047 g8; Naga_100649 g1 and Naga_100035 g43.

The genome of Phaeodactylum contains 5 genes annotated as ACS or ACL in the Phaeodactylum genome internet portal (http://protists.ensembl.orq/Phaeodactylum tricornutum): Phatr3_J20143 (gene named ACS1), Phatr3_J12420 (ACS2), Phatr3_J54151 (ACS3), Phatr3_J45510 (ACS4) and Phatr3_J17720 (ACL1).

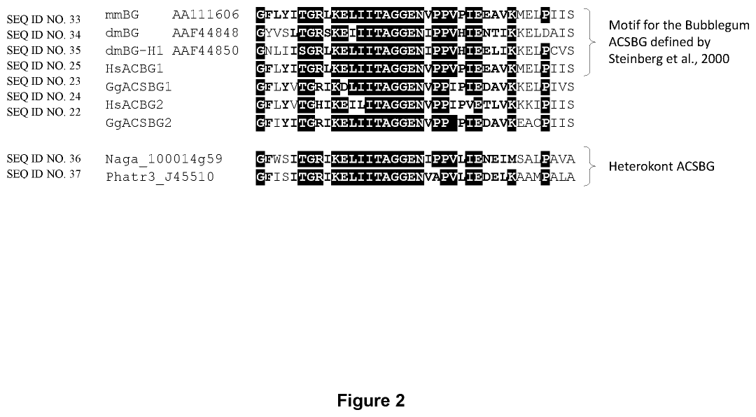

ACSBG proteins contain conserved motifs, including one motif initially described by (Steinberg et al., 2000), since then considered to be involved in fatty acid binding. This initial motif is shown in FIG. 2 and referred to here as “Motif II”, and which corresponds to the following sequence GXXXXTGRXKELIITAGGENXPPVXIEXXXKXXXP (SEQ ID NO. 1), wherein X is any amino acid residue.

One candidate gene coding for an Acyl coA synthase isoform in Nannochloropsis gaditana harboring this motif (Naga_100014 g59, herein referred to as NgACSBG and having amino acid sequence SEQ ID NO. 7) was identified by bioinformatic analysis using the http://www.Nannochloropsis.orq website. Likewise, one candidate gene coding for an Acyl CoA synthase isoform harboring this motif (annotated as ACS4/Phatr3_J45510, and referred to herein as PtACSBG, having amino acid sequence SEQ ID NO. 8) was identified in Phaeodactylum. The alignment of the 38-amino acid motif in NgACSBG and HsACSBG1 shows 68% of identical residues. The alignment of the 38-amino acid motif in PtACSBG and HsACSBG1 shows 71% of identical residues.

In addition, five conserved motifs of ACSBGs defined by (Lopes-Marques et al., 2018, Gene 664, 111-118) are also present in NgACSBG and PtACSBG, further assessing their structural definition as ACSBG sequences (alignment not shown).

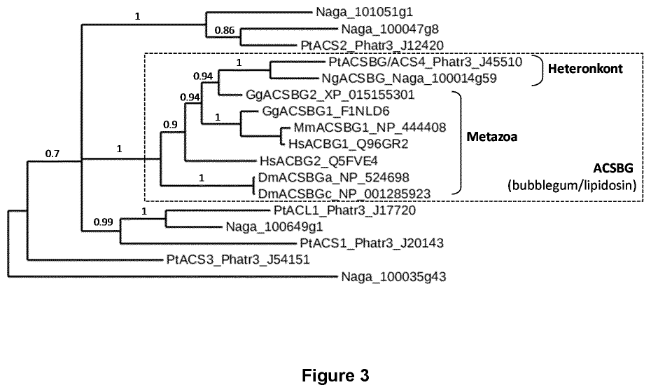

All the ACS sequences found in Nannochloropsis (Naga_100014 g59 (NgACSBG); Naga_100012 g66; Naga_101051 g1; Naga_100047 g8; Naga_100649 g1 and Naga_100035 g43) and Phaeodactylum (Phatr3_J20143 (ACS1), Phatr3_J12420 (ACS2), Phatr3_J54151 (ACS3), Phatr3_J45510 (PtACSBG or ACS4) and Phatr3_J17720 (ACL1)) were compared with sequences of previously characterized ACSBG in animals, i.e. Drosophila melanogaster (DmACSBGa, genbank reference NP_524698; DmACSBGc, genbank reference NP_001285923); Homo sapiens (HsACBG1, Uniprot reference Q96GR; HsACBG2, Uniprot reference Q5FVE4); Mus musculus or mouse (MmACSBG1, genbank reference NP_444408); Gallus gallus or chicken (GgACSBG1, Uniprot reference F1NLD6; GgACSBG2, genbank reference XP_015155301). To that purpose, a phylogenetic tree was reconstructed after a multiple alignment of sequences using the MUSCLE method (v3.8.31) configured for high accuracy with default settings (Edgar, 2004), removal of gaps and poorly aligned sequences using the Gblock method (Castresana, 2000). The tree was calculated using the www.phyloqeny.fr internet platform (Dereeper et al., 2008) using a bayesian inference method implemented in the MrBayes program (v3.2.6), with a number of substitution types fixed to 6, a Poisson model for amino acid substitutions, four Markov chain Monte Carlo (MCMC) chains run for 10,000 generations, sampling every 10 generations and a 50% majority rule consensus tree (Huelsenbeck and Ronquist, 2001; Ronquist et al., 2012). The obtained tree (FIG. 3) highlights the clear difference between ACSBG sequences from Nannochloropsis and Phaeodactylum belonging to the bubblegum/lipidosin group and all other ACS sequences from these microalgae.

The Heterokont ACS of the Bubblegum family (or ACSBG) can therefore be defined by the specific conservation of Motif II, as well as by high levels of similarity. When compared using the Blast software, an e-value <10⁻¹⁰ is obtained when comparing NgACSBG or PtACSBG with HsACSBG2.

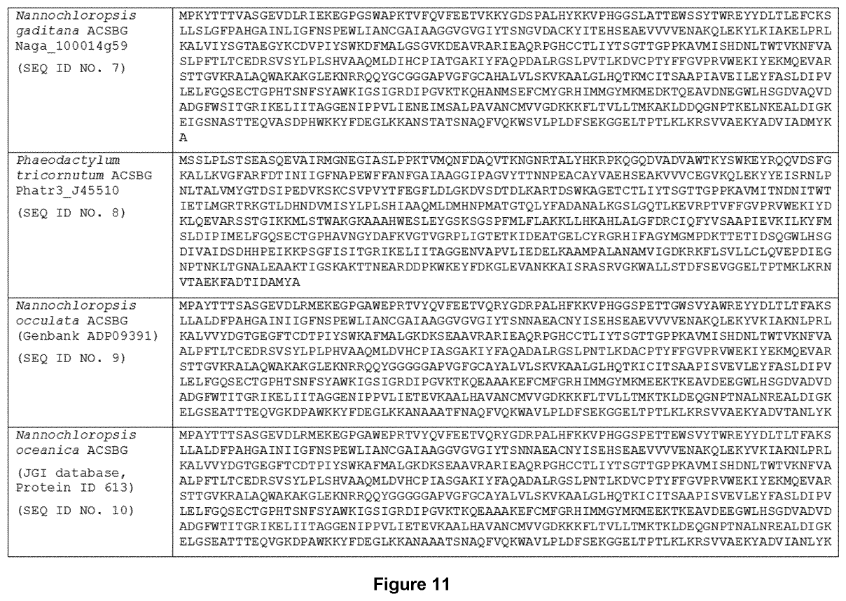

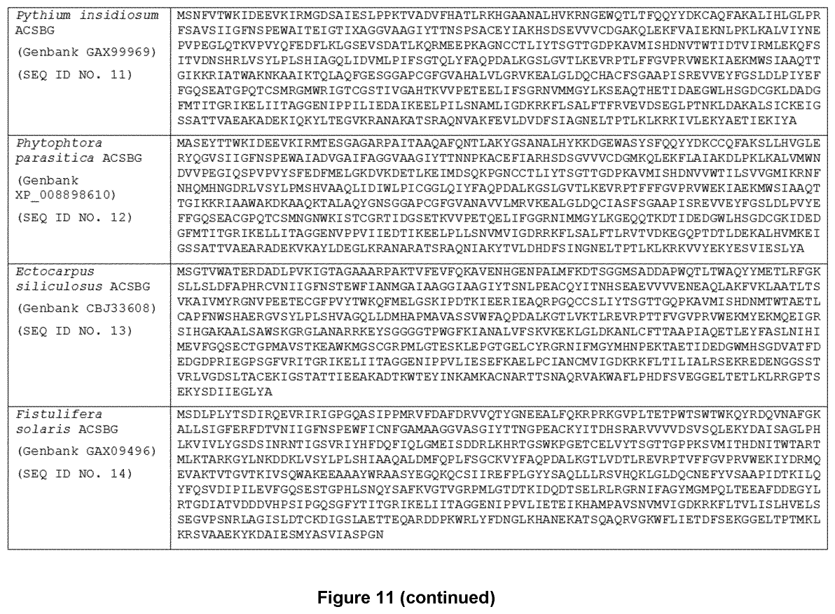

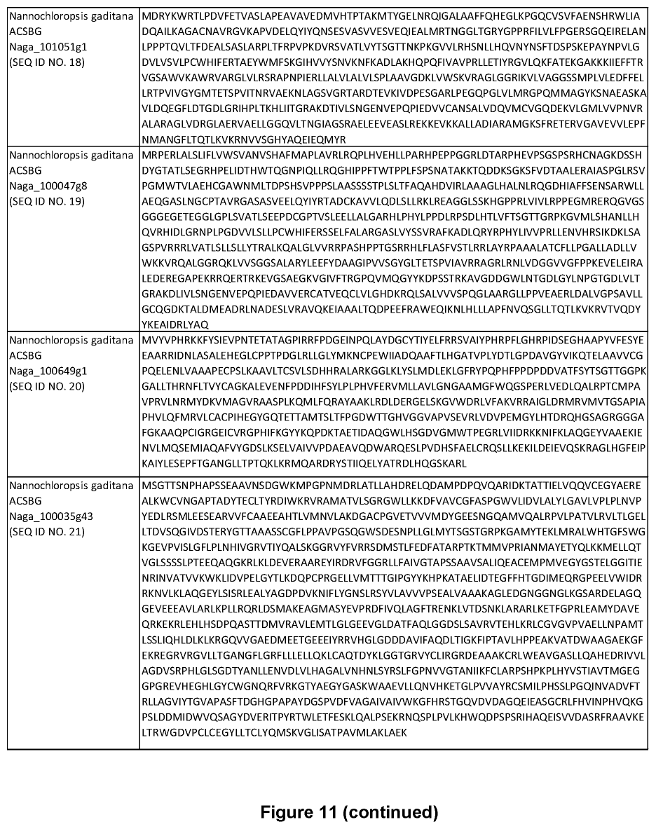

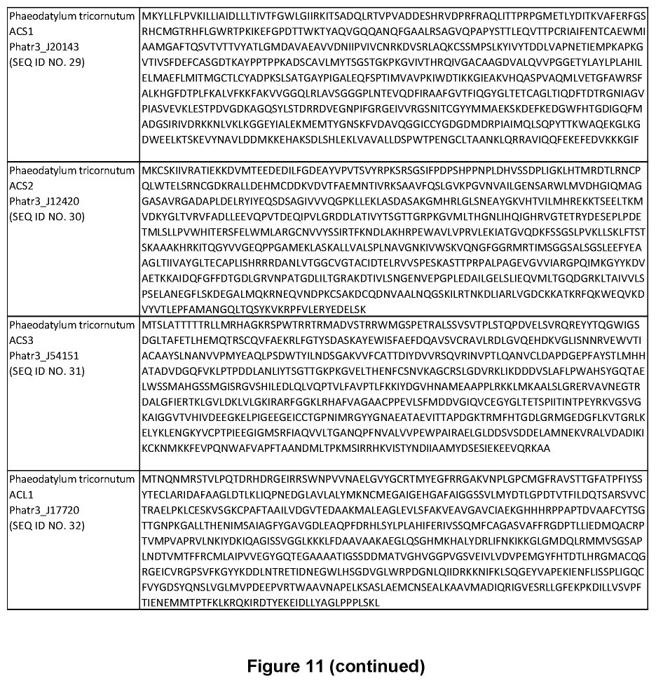

In line with this conclusion, other ACSBG sequences were identified in other Heterokonts and in other Nannochloropsis species. They are listed in FIG. 11. In particular, a search of highly similar sequences in other Nannochloropsis species, using the Blast method with default settings led to the identification of genes with an e-value <10⁻¹⁰, indicating a very high level of conservation in Nannochloropsis occulata (genbank reference ADP09391) and Nannochloropsis oceanica (JGI database, CCMP1779|4677-mRNA-1, Protein ID 613, genome location nanno_1077:101526-103472; https://qenome.jqi.doe.qov/cqi-bin/dispGeneModel?db=Nanoce1779&id=613). A search of highly similar sequences in other Heterokonta species, using the Blast method with default settings (Altschul et al., 1990) led to the identification of genes with an e-value <10⁻¹°, indicating a very high level of conservation in species of the following genera: Pithyum (genbank GAX99969), Phytophtora (genbank XP_008898610), Ectocarpus (genbank CBJ33608), Fistulifera (genbank GAX09496).

The NgACSBG sequence used in Example 2 below represents therefore an ACSBG gene found in other Nannochloropsis species, and in other Heterokonts, with a high level of conservation (Blast e-value<10⁻¹⁰, and the conservation of Motif II).

### Example 2: Point Mutation of an Endogenous ACSBG Gene Triggers TAG Accumulation in Nannochloropsis gaditana

Point mutations were introduced in Nannochloropsis gaditana using a specifically designed TALE-N nuclease (Daboussi et al., 2014, Nat. Commun 5, 3831). The region of the NgACSBG gene selected for the TALE-N driven mutation was selected by comparison with the whole genome so as to avoid binding of the TALE-N to another location in the genome.

Material and Methods

Constructions

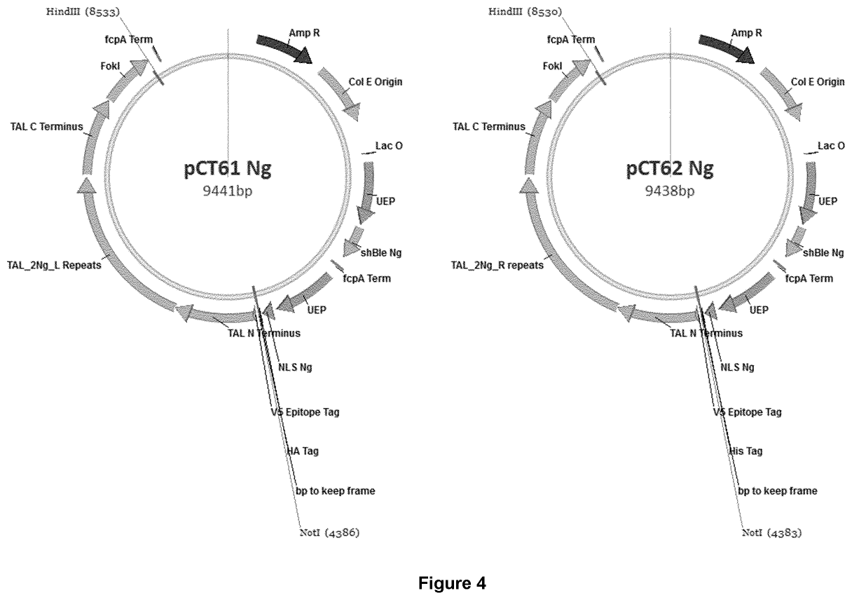

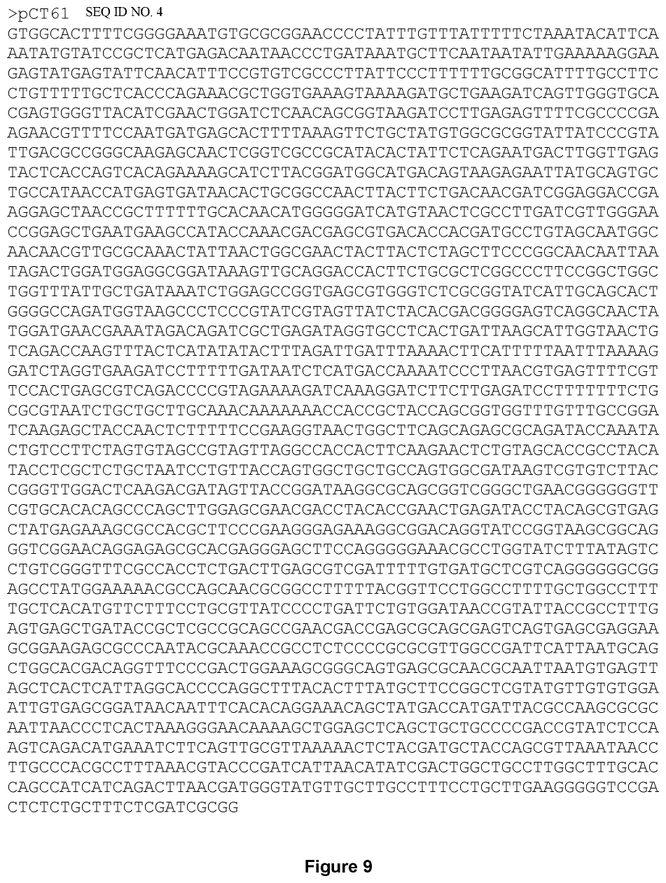

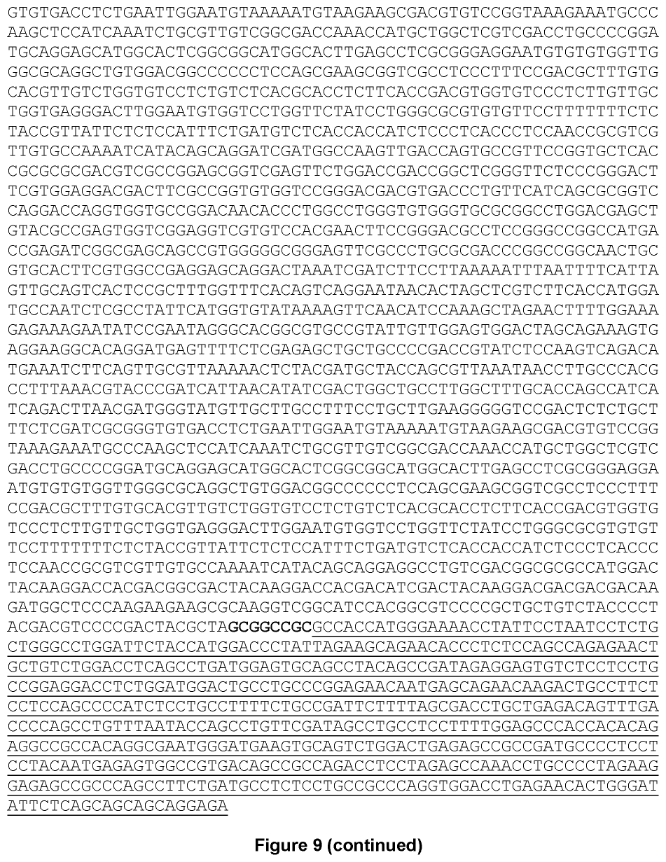

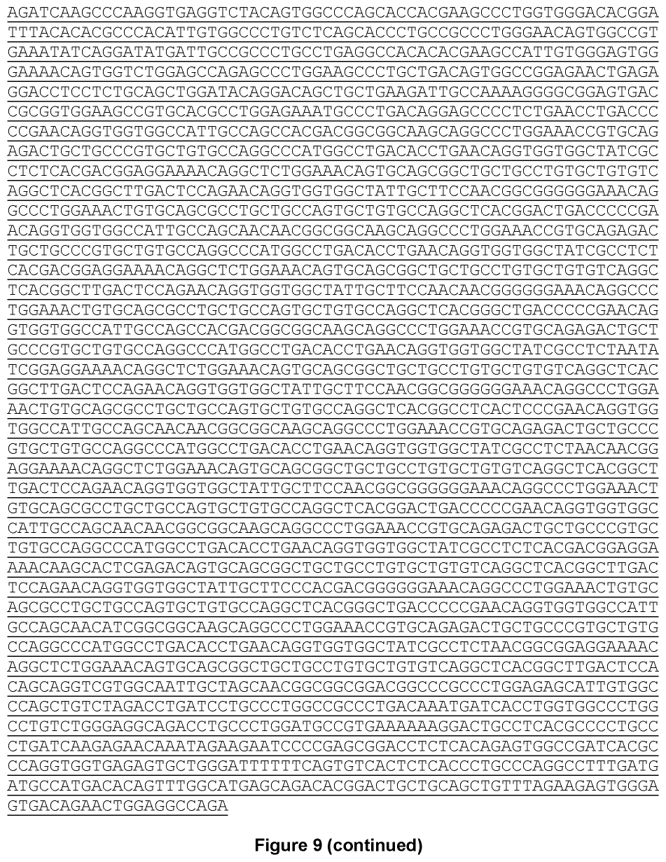

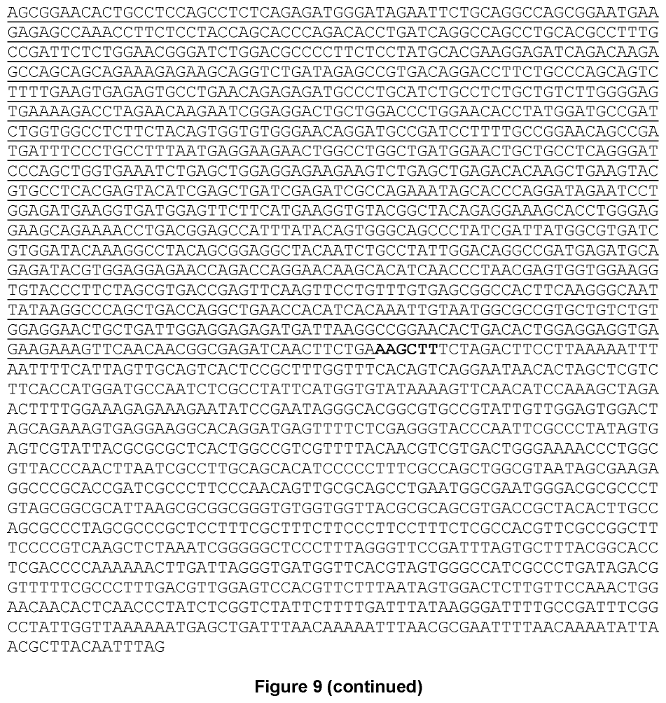

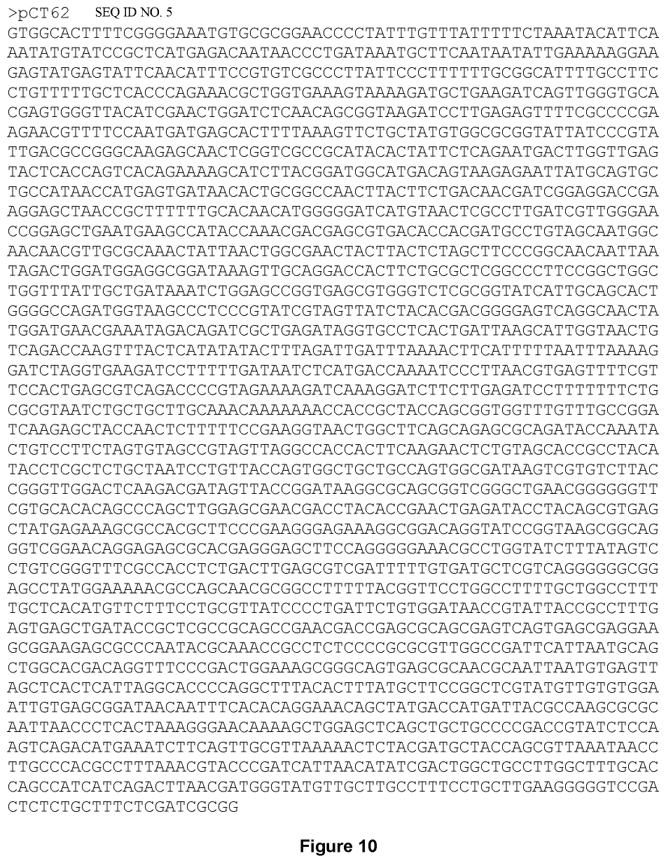

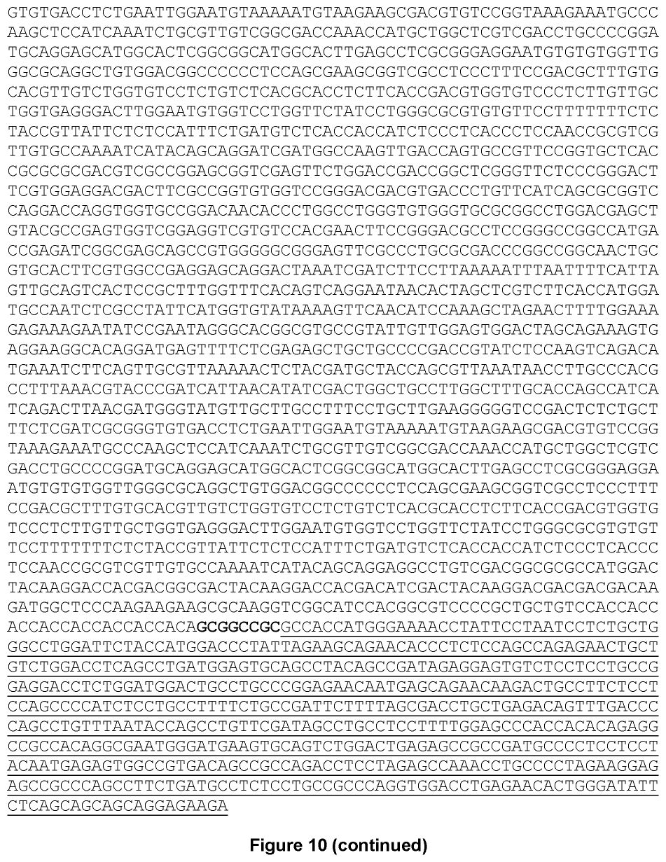

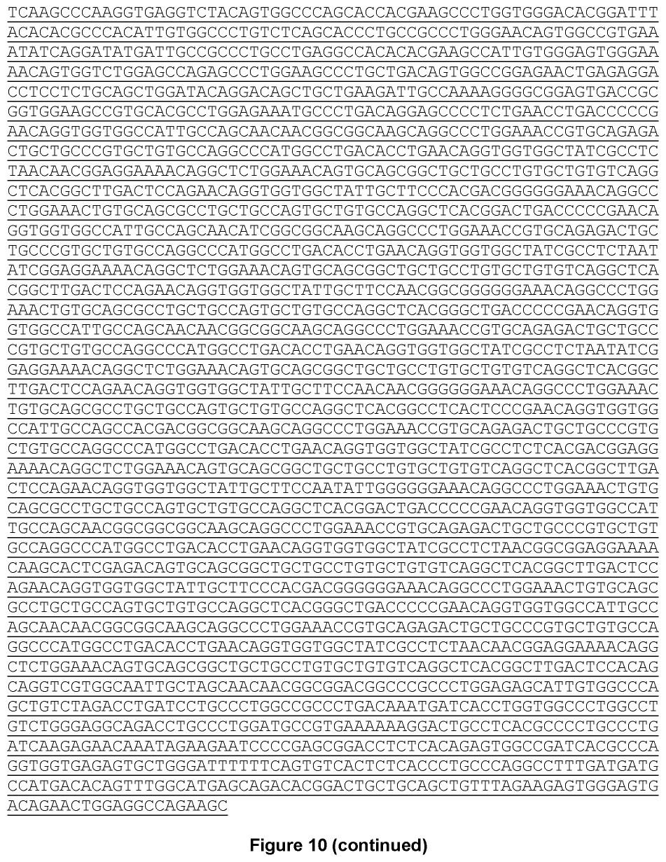

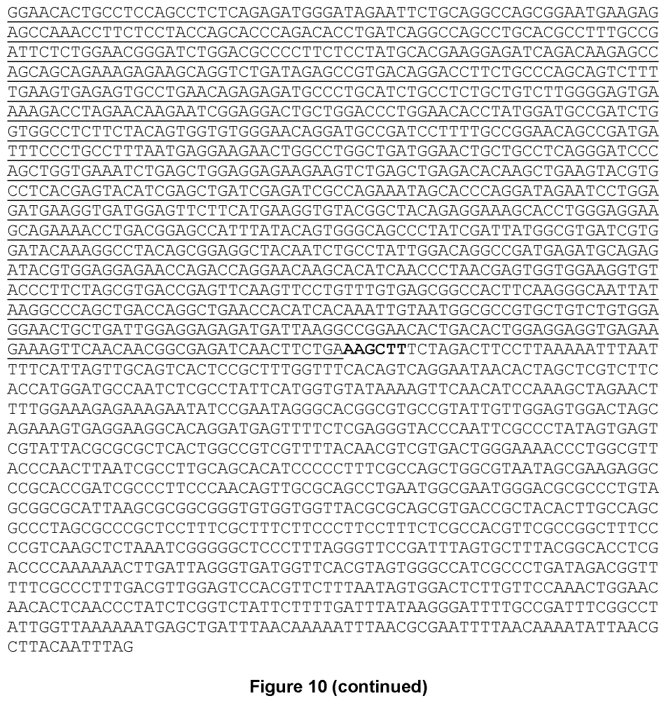

The synthesized Tal2Ng-L subunit binds to TCCTGCGCATGGTGCCATT (SEQ ID NO. 2) corresponding to Repeat Variable Diresidues (RVD) (T)-HD-HD-NG-NN-HD-NNHD-NI-NG-NN-NN-NG-NN-HD-HD-NI-NG-NG, whereas Tal2Ng-R subunit binds specifically to TGGCAATGAGCCATTCGGG (SEQ ID NO. 3) corresponding to RVD (T)-NN-NN-HD-NI-NI-NGNN-NI-NN-HD-HD-NI-NG-NG-HD-NN-NN-NN. In these RVD sequences, ‘(T)’ indicates that the first binding repeat is provided by the vector. Subcloning of Tal2Ng-L into pCT5Ng and Tal2Ng-R into pCT6Ng generates plasmids pCT61 and pCT62, respectively. Schematic representation of pCT61 and pCT62 is given in FIG. 4. The pCT61 sequence (SEQ ID NO. 4) is shown in FIG. 9. The pCT62 sequence (SEQ ID NO. 5) is shown in FIG. 10.

Nuclear Transformation of N. Gaditana

Plasmids pCT61 and pCT62 were linearized by digestion with ScaI and column-purified by the NucleoSpin® Gel and PCR Clean-up kit (Macherey-Nagel) following manufacturer's instructions. Two micrograms (one microgram TALE-N left subunit plasmid+one microgram TALE-N right subunit plasmid) linearized plasmids were electroporated into Nannochloropsis gaditana following the protocol published by (Radakovits et al., 2012, Nat Commun 3, 686). Transformed lines were selected on f/2 plates containing 7 μg mL⁻¹ zeocin. Integration of both TALE-N subunits was assessed by colony PCR with primers TAL-Nterm-Rev (GCAGGTCGCTAAAAGAATCG) (SEQ ID NO. 6) and TAL-HA Fw (CCCCGACTACGCTAGCG) (SEQ ID NO.41) for TALEN left subunit and TAL-Nterm-Rev (GCAGGTCGCTAAAAGAATCG) (SEQ ID NO.42) and TAL-His Fw (CACCACCACCACCACAGC) (SEQ ID NO.43) for TALEN right subunit.

T7 Endonuclease Test and Genetic Target Sequencing

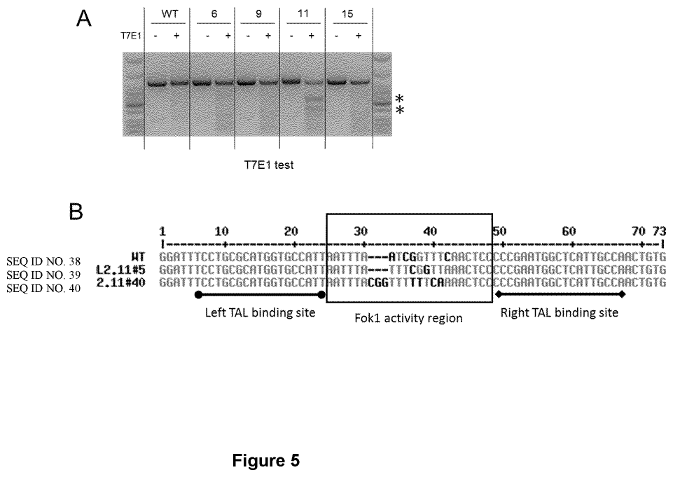

Few clones harboring both TALEN subunits were analyzed by T7E1 test to assess occurrence of genome editing at the target locus. To this aim, a fragment of around 396 bp where the FokI activity region is placed asymmetrically from the 5′ and 3′ ends was PCR-amplified from the positive colony with a proofreading polymerase. Bands corresponding to the desired products were purified from gel and quantified. For each clone, 1.3 μg of PCR product were treated with T7E1 (+) or left untreated (−). A WT was also included as a negative control. T7E1 reaction was performed following manufacturer's instruction, with minor modifications. One microliter of T7E1 enzyme was used in each reaction and incubation performed at 37° C. for one hour. Separation of the obtained fragments on 2% agarose gels highlighted the occurrence of mutations at target sites in some of the clones tested (FIG. 5A). Positive colonies were sub-cloned onto a new selective plate in order to separate cells harboring the mutation from those that presented a WT-like sequence. Mutation at NgACSBG target sites was confirmed by sequencing of the DNA at target regions (FIG. 5B).

Cultivation for Transformation:

Nannochloropsis gaditana Lubian Strain CCMP526 (Culture Collection of Marine Phytoplankton, now known as NCMA: National Center for Marine Algae and Microbiota) was used in all experiments. N. gaditana was grown at 20° C. in 250 mL flask in artificial seawater (ESAVV) medium (Table 1) using ten times enriched nitrogen and phosphate sources (5.49·10⁻³ M NaNO₃ and 2.24·10⁻⁴ NaH₃PO₄) called “10× ESAW”, or nitrogen-depleted medium, where NaNO₃was omitted. Cells were grown on a 12:12 light (60 μE m⁻² sec⁻¹)/dark cycle. For nuclear transformation, cells were grown under constant light in f/2 medium (Table 2) until they reached the late exponential phase. All cultures were maintained on f/2 plates solidified with 1% agar under a 12:12 light/dark regime in presence (transformed strains) or absence (wild type strain) of the selective antibiotic zeocin (7 μg mL⁻¹). When needed, cells were counted using a LUNA™ Automated Cell Counter following manufacturer's instructions.

TABLE 1
Composition of the “10x ESAW” cultivation medium
10X ESAW Medium
Final Concentration (mM)

| | |
|---|---|
| NaCl | 363 |
| Na₂SO₄ | 25 |
| KCl | 8.035 |
| NaHCO₃ | 2.071 |
| KBr | 0.725 |
| H₃BO₃ | 0.372 |
| NaF | 0.667 |
| MgCl₂ · 6H₂O | 47.172 |
| CaCl₂ · 2H₂O | 9.142 |
| SrCl₂ · 6H₂O | 0.082 |
| NaNO₃ | 5.49 |
| NaH₂PO₄ | 0.224 |
| Na₂ EDTA · 2H₂O | 0.0083 |
| Fe-EDTA | 0.00655 |
| CuSO₄ · 5H₂O | — |
| Zn SO₄ · 7H₂O | 0.000254 |
| CoSO₄ · 7H₂O | 0.0000569 |
| MnSO₄ · 7H₂O | 0.00242 |
| Na₂MoO₄ | — |
| biotin (vit.H) | 4.093E−06 |
| Cobalamin (Vit.B12) | 7.378E−07 |
| thiamine (vit.B1) | 0.000665 |

TABLE 2
Composition of the “f/2” cultivation medium
f/2 Medium
Final Concentration (mM)

| | |
|---|---|
| Tris pH8 | 40 |
| NaCl | 363 |
| Na₂SO₄ | 25 |
| KCl | 8.035 |
| NaHCO₃ | 2.071 |
| KBr | 0.725 |
| H₃BO₃ | 0.372 |
| NaF | 0.667 |
| MgCl₂ · 6H₂O | 47.172 |
| CaCl₂ · 2H₂O | 9.142 |
| SrCl₂ · 6H₂O | 0.082 |
| NaNO₃ | 0.882 |
| NaH₂PO₄ | 0.0417 |
| Na₂ EDTA · 2H₂O | 0.0117 |
| FeCl₃ · 6H₂O | 0.0117 |
| CuSO₄ · 5H₂O | 4.0048E−05 |
| Zn SO₄ · 7H₂O | 7.652E−05 |
| CoCl₂ · 6H₂O | 4.203E−05 |
| MnCl₂ · 2H₂O | 0.00111 |
| Na₂MoO₄ | 3.0597E−05 |
| biotin (vit.H) | 4.0932E−06 |
| Cobalamin (Vit.B12) | 7.378E−07 |
| thiamine (vit.B1) | 0.000665 |

Cultivation for Growth in Erlenmeyers:

Liquid cultures of Nannochloropsis were inoculated at 2.5.10⁶ cells/ml into ESAW-10N or ESAW-ON, and cultivated at 20° C., 100 RPM in 12:12 light:dark cycle (illumination at 60 μE M⁻² sec⁻¹) for 7 days. Cells were counted using a LUNA™ Automated Cell Counter following manufacturer's instructions, and harvested by centrifugation at 3,500×g for 10 minutes. Cells were immediately frozen in liquid nitrogen and then stored at −80° C.

Cultivation for Growth with CO₂ Supply

Liquid cultures of Nannochloropsis were inoculated at 2.5.10⁶ cells/ml into ESAW-10N and cultivated for 3 days in small photobioreactors (Multi-Cultivator MC 1000, Photon Systems Instruments, Czech Republic) at 24° C., in continuous light (illumination at 60 μE M⁻² sec⁻¹). Culture mixing throughout cultivation time was provided by gas sparging in which CO₂ levels were maintained constant at 0.5% as in air-lift photobioreactors. Precise and constant CO₂ supplies to bioreactor tubes were provided by the Gas Mixing System GMS 150 (Photon Systems Instruments, Czech Republic) following manufacturer's instructions. Cells were counted using a LUNA™ Automated Cell Counter following manufacturer's instructions, and harvested by centrifugation at 3,500×g for 10 minutes. Cells were immediately frozen in liquid nitrogen and then stored at −80° C.

Lipid Assessment

Glycerolipids were extracted from freeze-dried Nannochloropsis cells grown in 50 mL of medium. About 50 to 100.10⁶ cells are required for a triplicate analysis of TAGs. A freeze-dried pellet was suspended in 4 mL of boiling ethanol for 5 minutes to prevent lipid degradation, and lipids were extracted as described by (Simionato et al., 2013 Eukaryot Cell. 201, 665-76) by addition of 2 mL methanol and 8 mL chloroform at room temperature. The mixture was then saturated with argon and stirred for 1 hour at room temperature. After filtration through glass wool, cell debris was rinsed with 3 mL chloroform/methanol 2:1, v/v, and 5 mL of NaCl 1% were then added to the filtrate to initiate biphase formation. The chloroform phase was dried under argon before solubilizing the lipid extract in 1 ml of chloroform. Total glycerolipids were quantified from their fatty acids, in a 10 μl aliquot fraction a known quantity of 15:0 was added and the fatty acids present were transformed as methyl esters (FAME) by a 1 hour incubation in 3 mL 2.5% H2SO4 in pure methanol at 100° C. (Jouhet et al., 2003, FEBS Lett 544, 63-68). The reaction was stopped by addition of 3 mL water, and 3 mL hexane was added for phase separation. After 20 min of incubation, the hexane phase was transferred to a new tube. FAMEs were extracted a second time via the addition, incubation and extraction of another 3 ml hexane. The combined 6 ml were argon-dried and re-suspended in 30 μl hexane for gas chromatography-flame ionization detector (GC-FID) (Perkin Elmer) analysis on a BPX70 (SGE) column. FAME were identified by comparison of their retention times with those of standards (Sigma) and quantified by the surface peak method using 15:0 for calibration. Extraction and quantification were performed with at least three biological replicates. TAGs were analyzed and quantified by HPLC-MS/MS. For a technical triplicate analysis, an aliquot of the lipid extract containing 25 nmol of total fatty acid was dried under argon and dissolved in 100 μl of a methanol/chloroform solution (1:2) containing 125 pmol of 18:0/18:0/18:0 TAG as internal standard. For each replicate, 20 μl were injected in the HPLC-MS/MS system. The analytic device comprised a LC system with binary pumps (Agilent 1260 Infinity) coupled to a QQQ MS (Agilent 6460) equipped with a JetStream electrospray vane of injection. TAGs were separated by HPLC from other lipids using a diol column (Macherey-Nagel, EC 150/2 Nucleosil 100-5 OH) maintained at 40° C. The chromatography conditions were as follows: solvent A: isopropanol/water/ammonium acetate 1 M pH 5.3 (850/125/1); solvent B: Hexane/isopropanol/water/ammonium acetate 1 M pH 5.3 (625/350/24/1); gradient: 0 to 5 min 100% B, 5 to 30 min linear increase of A to 100%, 30 to 45 min 100% A, 45 to 50 min:linear increase of B to 100%, 50 to 70 min 100% B. Under these conditions, TAGs were eluted after 4-5 min of run. The various TAG species were detected from their m/z ratio by MS/MS using the Multiple Reaction Monitoring (MRM) mode. The various transition reactions used to identify the different TAG species are those previously established with Phaeodactylum tricornutum (Abida et al., 2015). Quantification was made using the Agilent Mass Hunter® software furnished by the MS supplier.

Results

Identification of Mutations in Potential Clones

Clones selected on plates were analyzed using T7 endonuclease activity assay in order to determine whether the TALE-nuclease had been active at target site. As shown in FIG. 5A, clone 11 showed additional bands at low molecular weight (indicated by stars) and harbored therefore a mutation at the target site.

To define precisely how the TALE-nuclease modified the target DNA site, cells from this colony were sub-cloned onto a new selective plate in order to separate cells harboring the mutation from those that presented a WT-like sequence. Mutation at NgACS target sites was confirmed by sequencing of the DNA at target regions for 2 sub-clones, vsc 5 and vsc 40 (FIG. 5B). Both clones showed in-frame deletions (meaning that the RNA encodes a protein with the same number of amino acids) and non-synonymous codons were introduced as a result of nuclease activity: Arginine encoded by CGG and Glutamine by CAA in Tal2.11VSC #40; Arginine encoded by CGG and Leucine encoded by TTA in Tal2.11VSC #5. Thus following TALE-nuclease action, the RNA sequence is modified leading to a change in the amino acid sequence of the NgACSBG protein.

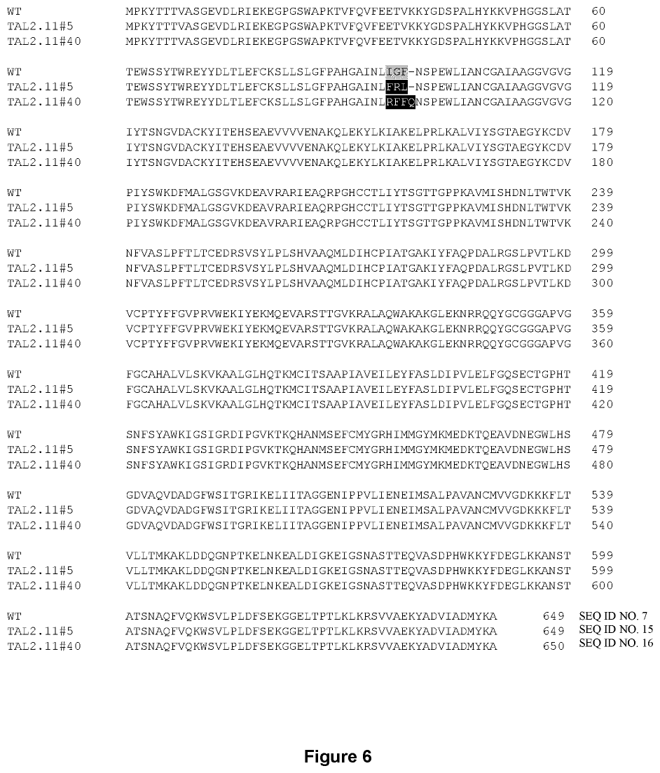

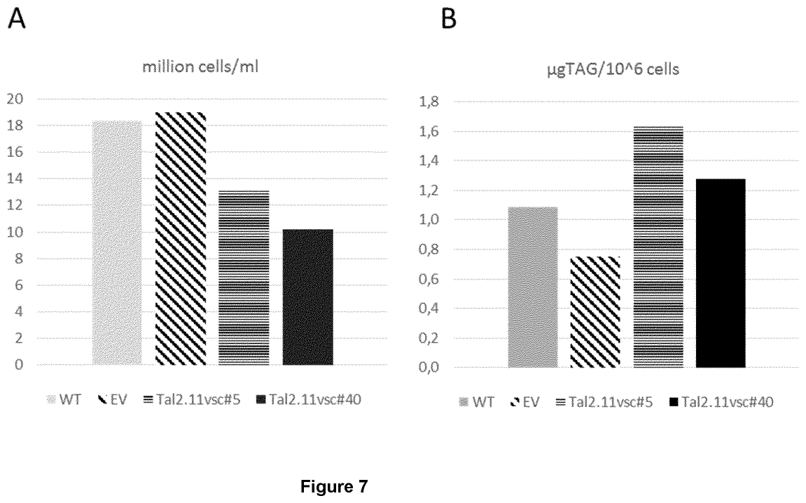

Two mutant lines were obtained harboring mutated endogenous ACSBG genes, called here NgACSBG #5 or TAL2.11vsc #5 and ACSBG #40 or TAL2.11vsc #40. Only two to three amino acids are modified in the endogenous proteins (FIG. 6).

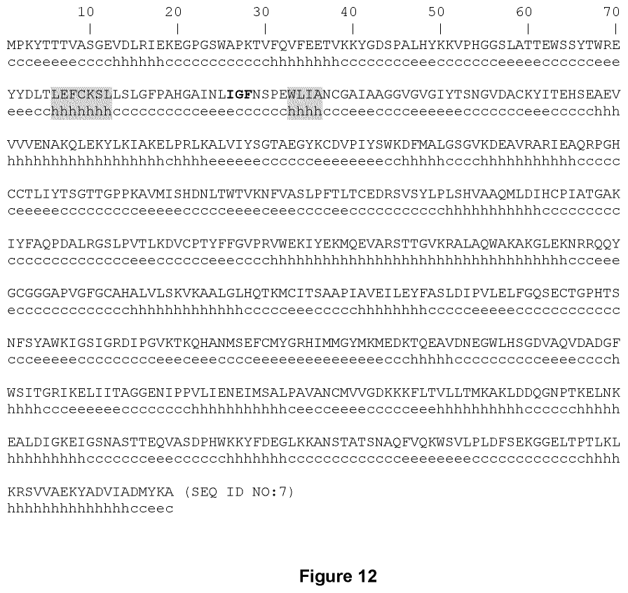

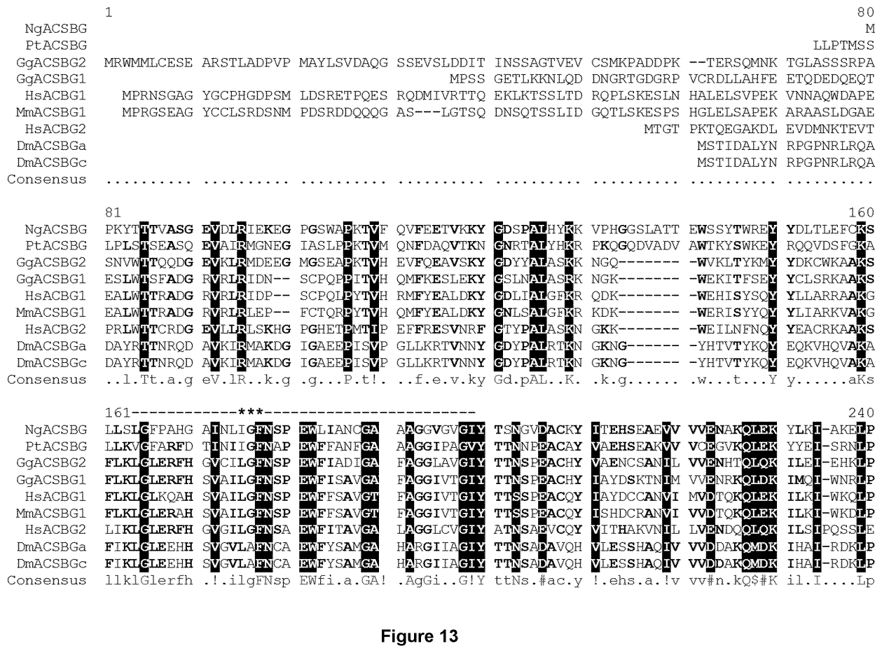

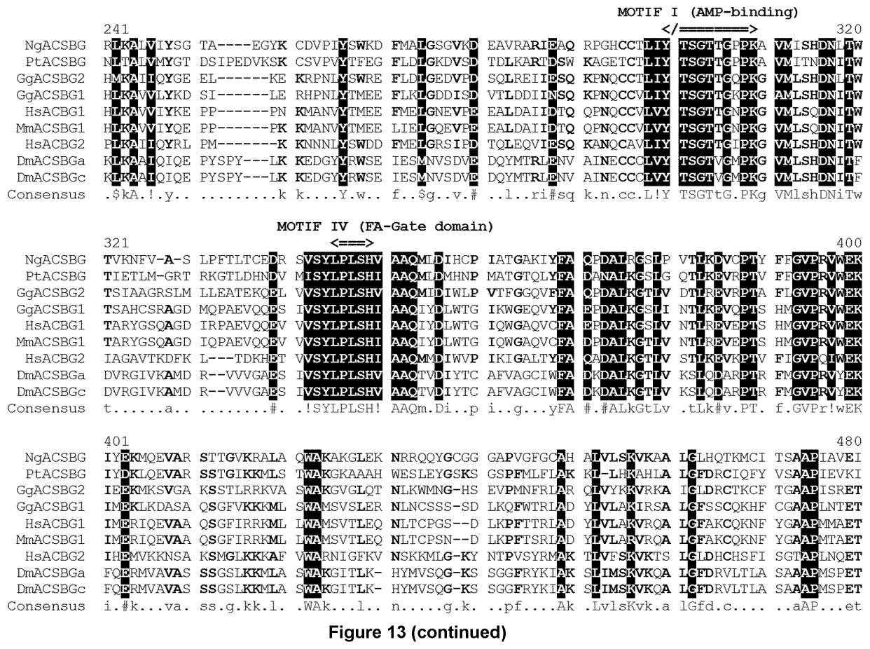

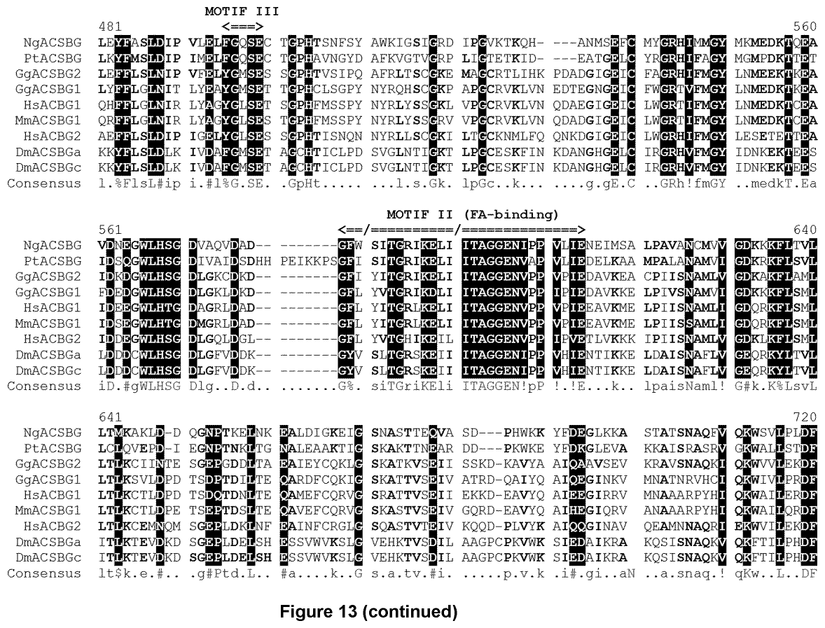

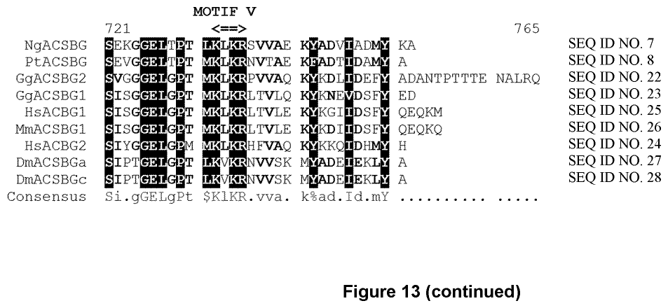

An analysis of the secondary structure of NgACSBG as predicted by the GORA method, revealed that the different mutations are all in the extended/coil domain located between two alpha helixes of NgACSBG (FIG. 12), corresponding to SEQ ID NO:17. Thus this region was identified as of particular interest to generate ACSBG mutations. However, it is expected that this region can be extended N- and C-terminally to a region spanning from amino acid 82 to 120 of SEQ ID NO:7, or corresponding regions in homologous ACSBG proteins.

Effect of Mutations on TAG Content

Cells were grown in 50 ml liquid medium supplemented with Nitrate. Growth of both mutants was affected (FIG. 7A), showing that the mutation had an impact on the physiology of the cell. From the cell pellets of these cultures, TAGs were extracted and quantified (FIG. 7B). Both mutants showed an improved TAG content by up to 1.6-fold compared to control strains.

This indicates that a point mutation in the NgACSBG sequence can allow a modification of fatty acid partitioning inside Nannochloropsis gaditana cells and lead to an accumulation of TAG.

Effect of Mutations on TAG Content with Supplied CO₂

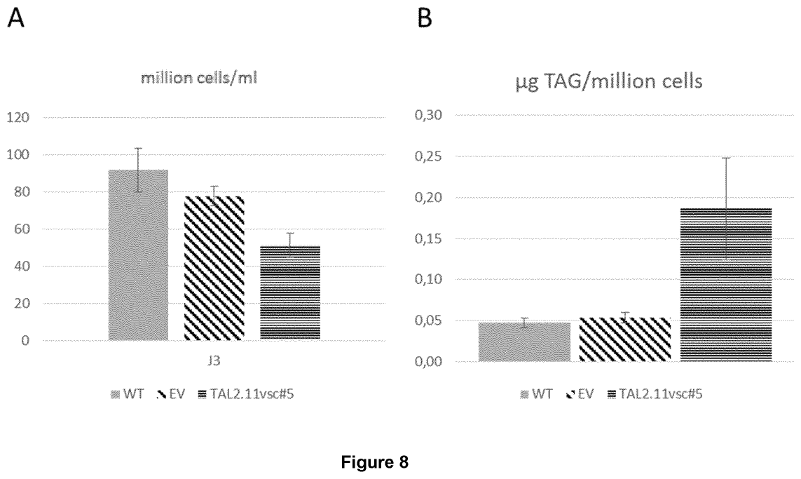

Cells were grown in air-lift photobioreactors bubbled with 0.5% CO₂ enriched air, in medium supplemented with nitrate. Growth of mutant was affected (FIG. 8A), showing that the mutation had an impact on the physiology of the cell. From the cell pellet of this culture, TAGs were extracted and quantified (FIG. 8B). Mutants showed an improved TAG content by up to 4-fold compared to control strains.

This indicates that when Nannochloropsis gaditana cells are provided with an excess of CO₂, carbon partitioning in the mutant is enhanced in favor of an increased TAG accumulation.

Sequences

In the sequence provided in FIGS. 9 and 10, NotI and HindIII sites used for subcloning are highlighted in bold, whereas TALEN subunits are underlined.

### Example 3: TALE-N Generation of Nannochloropsis gaditana acsbg Mutants

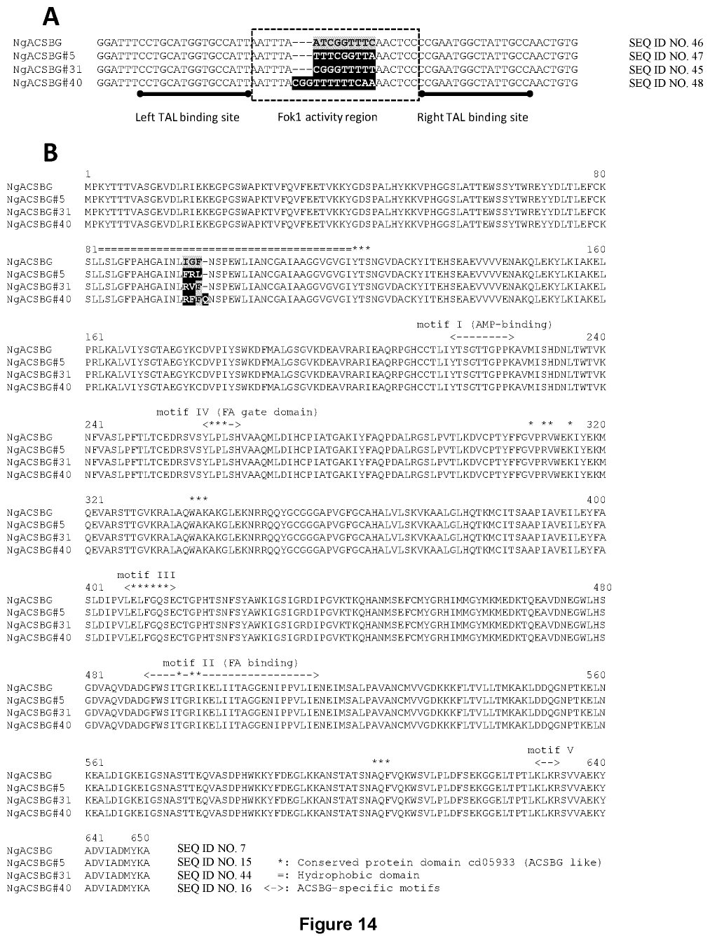

A third N. gaditana transformed line, NgACSBG #31, wherein TALE-N activity led to in-frame deletions and introduction of non-synonymous codons, leading to point mutant proteins (FIG. 14A, B), was generated as described in Example 2.

### Example 4: In Vivo Impact of NgACSBG #5 and NgACSBG #31 In-Frame Mutations on Glycerolipid Profiles

Cells were cultivated in parallel in a nutrient-rich liquid medium, using a Multicultivator photobioreactor, supplied with CO2 as described in Example 2. Control lines consisted of untransformed cells (wild type, WT) and cells transformed with an empty vector (EV), without any TALE-N sequence. The Multicultivator system allows the cultivation of 8 tubes in parallel: experiments were performed in duplicate for each line and were repeated to obtain data from independent replicates (n=4). Glycerolipids were analyzed as follows: Glycerolipids were extracted from freeze-dried N. gaditana cells grown in 50 mL of indicated medium. About 50 to 100×10⁶ cells were required for each triplicate analysis. A freeze-dried pellet was suspended in 4 mL of boiling ethanol for 5 minutes to prevent lipid degradation, and lipids were extracted as described earlier (Simionato et al., 2013. Eukaryot Cell 12: 665-676), by addition of 2 mL methanol and 8 mL chloroform at room temperature. The mixture was saturated with argon and stirred for 1 hour at room temperature. After filtration through glass wool, cell debris were rinsed with 3 mL chloroform/methanol 2:1, v/v, and 5 mL of NaCl 1% were then added to the filtrate to initiate biphase formation. The chloroform phase was collected, dried under argon before solubilizing the lipid extract in 1 ml of chloroform. Total glycerolipids were quantified based on their fatty acid (FA) content: in a 10 μl aliquot fraction, a known quantity of 15:0 was added and FAs were converted into FA methyl esters (FAME) by a 1 hour incubation in 3 mL 2.5% H2SO4 in pure methanol at 100° C. (Jouhet et al., 2003. FEBS Lett 544: 63-68). The reaction was stopped by addition of 3 mL water, and 3 mL hexane were added for phase separation. After 20 min incubation, the hexane phase was transferred to a new tube. FAMEs were extracted a second time via the addition, incubation and extraction of another 3 ml hexane. The combined 6 ml were argon-dried and re-suspended in 30 μl hexane for gas chromatography-flame ionization detector (GC-FID) (Perkin Elmer) analysis on a BPX70 (SGE) column. FAMEs were identified by comparison of their retention times with standards (Sigma) and quantified by the surface peak method using 15:0 for calibration. Extraction and quantification were performed with at least three biological replicates. Glycerolipids were then analyzed and quantified by high pressure liquid chromatography-tandem mass spectrometry (HPLC-MS/MS), with appropriate standard lipids. The lipid extracts corresponding to 25 nmol of total fatty acids were dissolved in 100 μL of chloroform/methanol [2/1, (v/v)] containing 125 pmol of each internal standard. Internal standards used were phosphatidylethanolamine (PE) 18:0-18:0 and diacylglycerol (DAG) 18:0-22:6 from Avanti Polar Lipid, and sulfoquinovosyldiacylglycerol (SQDG) 16:0-18:0 extracted from spinach thylakoid (Deme et al., 2014. FASEB J 28: 3373-3383) and hydrogenated (Buseman et al., 2006. Plant Physiol 142: 28-39). Lipids were then separated by HPLC and quantified by MS/MS. The HPLC separation method was adapted from previously described procedure (Rainteau et al., 2012. PLoS One 7: e41985). Lipid classes were separated using an Agilent 1200 HPLC system using a 150 mm×3 mm (length×internal diameter) 5 μm diol column (Macherey-Nagel), at 40° C. The mobile phases consisted of hexane/isopropanol/water/1 M ammonium acetate, pH 5.3 [625/350/24/1, (v/v/v/v)] (A) and isopropanol/water/1 M ammonium acetate, pH 5.3 [850/149/1, (v/v/v)] (B). The injection volume was 20 μL. After 5 min, the percentage of B was increased linearly from 0% to 100% in 30 min and kept at 100% for 15 min. This elution sequence was followed by a return to 100% A in 5 min and an equilibration for 20 min with 100% A before the next injection, leading to a total runtime of 70 min. The flow rate of the mobile phase was 200 μL/min. The distinct glycerophospholipid classes were eluted successively as a function of the polar head group. Mass spectrometric analysis was performed on a 6460 triple quadrupole mass spectrometer (Agilent) equipped with a Jet stream electrospray ion source under following settings: drying gas heater at 260° C., drying gas flow at 13 L·min-1, sheath gas heater at 300° C., sheath gas flow at 11 L·min-1, nebulizer pressure at 25 psi, capillary voltage at ±5000 V and nozzle voltage at ±1,000 V. Nitrogen was used as collision gas. The quadrupoles Q1 and Q3 were operated at widest and unit resolution respectively. Phosphatidylcholine (PC) and diacylglyceryl hydroxymethyltrimethyl-β-alanine (DGTS) analyses were carried out in positive ion mode by scanning for precursors of m/z 184 and 236 respectively at a collision energy (CE) of 34 and 52 eV. SQDG analysis was carried out in negative ion mode by scanning for precursors of m/z −225 at a CE of −56 eV. PE, phosphatidylinositol (PI), phosphatidylglycerol (PG), monogalactosyldiacylglycerol (MGDG) and digalactosyldiacylglycerol (DGDG) measurements were performed in positive ion mode by scanning for neutral losses of 141 Da, 277 Da, 189 Da, 179 Da and 341 Da at CEs of 20 eV, 12 eV, 16 eV, 8 eV and 8 eV, respectively. DAG and triacylglycerol (TAG) species were identified and quantified by multiple reaction monitoring (MRM) as singly charged ions [M+NH4]+ at a CE of 16 and 22 eV respectively. Quantification was done for each lipid species by multiple reaction monitoring (MRM) with 50 ms dwell time with the various transitions previously recorded (Abida et al., 2015. Plant Physiol 167: 118-136). Mass spectra were processed using the MassHunter Workstation software (Agilent) for identification and quantification of lipids. Lipid amounts (pmol) were corrected for response differences between internal standards and endogenous lipids as described previously (Jouhet et al., 2017. PLOS ONE 12: e0182423).

Results

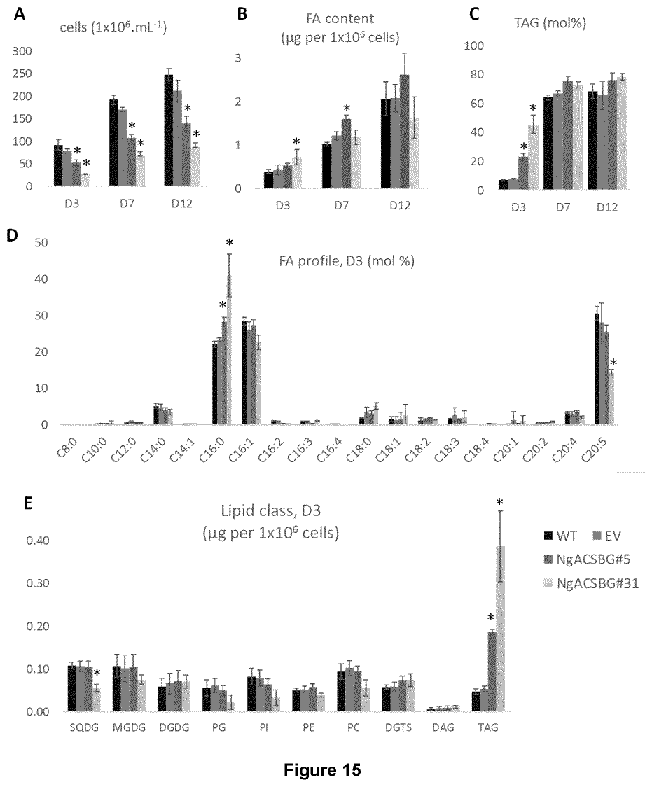

The NgACSBG #5 and NgACSBG #31 lines showed a slower growth compared to WT and EV lines, monitored by cell counting at day 3, 7 and 12 following inoculation (D3, D7 and D12, respectively) (FIG. 15, A). Compared to WT and EV lines, the total FA content per cell was higher in NgACSBG #5 and NgACSBG #31 mutants in the early stage of cultivation (D3) (FIG. 15, B), reflecting a higher content in TAG (FIG. 15, C). WT and EV showed an accumulation of TAG at D7 and D12 induced by the shortage of nutrients in the medium (FIG. 15, C). NgACSBG #5 and NgACSBG #31 mutants showed also an increased TAG content at D7 and D12, but the difference with WT and EV lines was less pronounced.

We focused our comparison of NgACSBG #5 and NgACSBG #31 with WT and EV cells at D3. The total FA profile of mutant lines showed an increase in 16:0 proportion balanced by a decrease of 20:5 (FIG. 15, D). The glycerolipid profile highlighted an increase in TAG and little change in the level of other glycerolipid classes (FIG. 15, E). Since in N. gaditana TAG are 16:0-rich and 20:5-poor (Simionato et al., 2013. Eukaryot Cell 12: 665-676; Alboresi et al., 2016. Plant Physiol 171(4):2468-82), one could consider that the increase in 16:0 and the decrease in 20:5 in the mutant lines might simply reflect the accumulation of TAG.

### Example 5: Molecular Impact of NgACSBG #5 and NgACSBG #31 Mutations on the Protein Structure

Materials and Methods

Modeling of NgACSBG Structure, Bound to Coenzyme a, Alpha-Linolenic Acid (18:3) and AMP

Structure modeling was based on the identification of acyl-CoA synthetases conserved motifs, secondary structure predictions and alignments with homologous sequences, which protein 3D structures have been resolved and stored in the Protein Data Bank (PDB, (Burley et al., 2019. Nucleic Acids Research 47: D520-D528)). Motif predictions were performed using online servers MOTIF (Kanehisa et al., 2002. Nucleic Acids Res 30: 42-46), PROSITE (Sigrist et al., 2013. Nucleic Acids Res 41: D344-347) and Superfamily (Wilson et al., 2009. Nucleic Acids Research 37: D380-D386). Secondary structures were predicted using the Porter (Mirabello and Pollastri, 2013. Bioinformatics 29: 2056-2058) and Predictprotein (Yachdav et al., 2014. Nucleic Acids Res 42: W337-343) online tools. Search for homologous sequences was performed using Blast (Altschul et al., 1990. Journal of Molecular Biology 215: 403-410). Automated structure prediction was attempted using Robetta (Song et al., 2013. Structure 21: 1735-1742), RaptorX (Kallberg et al., 2012. Nature Protocols 7: 1511-1522), Swiss-Model (Waterhouse et al., 2018. Nucleic Acids Res 46: W296-W303), PhyRe2 (Kelley et al., 2015. Nat Protoc 10: 845-858), I-Tasser (Zhang, 2009. Proteins 77 Suppl 9: 100-113), PsiPred (Buchan et al., 2013. Nucleic Acids Res 41: W349-357) and PS(2) (Huang et al., 2015. Nucleic Acids Res 43: W338-342) servers.

Four templates were selected for NgACSB modeling: 1ULT, (a long chain fatty acyl-CoA synthetase homodimer from Thermus thermophiles) (Hisanaga et al., 2004); 1PG4 (acetyl-CoA synthetase from Salmonella enterica) (Gulick et al., 2003); 5MSC (the A domain of carboxylic acid reductase from Nocardia iowensis) (Gahloth et al., 2017. Nat Chem Biol 13: 975-981); 3EQ6, a human acyl-CoA synthetase medium-chain family member (Kochan et al., 2009. Journal of Molecular Biology 388: 997-1008). The sequences of these four template proteins were aligned with NgACSBG.

3D structural homology models were built with Modeller (Sali and Blundell, 1993. J Mol Biol 234: 779-815) for the full length NgACSBG protein, from residue M1 to A649. Modeller being able to use multiple structure alignments, four models were built using different association of template proteins: M1 μg4 from 1PG4 alone, M3eq6 from 3EQ6 alone and two consensus models Mcons1 from 1PG4, 1ULT and 5MSC and Mcons2 from all four protein templates.

Positioning of CoA and AMP in the models was based on their neighboring residues in structures 1PG4 and 3EQ6 (Tables 3 and 4) using a transformation matrix to orient these residues in the X-ray structure of 1PG4 towards their homologs in the models of NgACSBG. In brief, to position CoA, backbone atoms of residues 305, 356, 357, 360, etc. in 1PG4 were translated and rotated to minimize the root-mean-square (rms) deviation with residues 259, 310, 311, 314, etc. in the model of NgACSBG. The same transformation was applied on the CoA molecule present in 1PG4 to properly insert it in the models. An AMP molecule, also present in 1PG4, was positioned in the two models M1 pg4 and Mcons1, in the same way. Since 3EQ6 also contains both CoA and AMP, the positioning of CoA and AMP in models M3eq6 and Mcons2 was done using the matrix transformations pertaining to 3EQ6. As an example of fatty acid, α-linolenic acid (18:3; C₁₈H₃₀O₂ cis-Δ9, 12, 15) was used. The lipid was initially placed manually inside the structures, with head in the direction of AMP, respecting its proximity with CoA and profiting of hydrophobic holes seen in the structures.

TABLE 3
Aminoacids in NgACSBG sequence predicted to be
in the vicinity of Coenzyme A (CoA).

| | | | | | | | | | | |
|---|---|---|---|---|---|---|---|---|---|---|
| Naga | L259 | RV | K314 | G341 | AGGE | L538 | S574 | V579 | D582 | H584 |
| | | 310-311 | | | 504-507 | | | | | |
| | | | | | (SEQ ID | | | | | |
| | | | | | NO: 49) | | | | | |
| ACSBG2 | L283 | QI | K348 | G365 | AGGE | L566 | S603 | 1608 | D613 | L615 |
| | | 344-345 | | | 532-536 | | | | | |
| | | | | | (SEQ ID | | | | | |
| | | | | | NO: 50) | | | | | |
| 1PG4 | A305 | TA | A360 | G387 | VSGH | K555 | R584 | P589 | T592 | D594 |
| | | 356-357 | | | 522-525 | | | | | |
| | | | | | (SEQ ID | | | | | |
| | | | | | NO: 51) | | | | | |
| 3EQ6 | S262 | GA | V314 | G338 | SSGY | R501 | K532 | P537 | Y540 | R542 |
| | | 310 311 | | | 468-471 | | | | | |
| | | | | | (SEQ ID | | | | | |
| | | | | | NO: 52) | | | | | |

Abbreviations:
NAGA, NgACSBG; ACSBG2: long-chain-fatty acid-CoA ligase from Gallus gallus; 1PG4, Acetyl-CoA Synthetase from Salmonella enterica; 3EQ6, a human acyl-CoA synthetase medium-chain family member.

TABLE 4
Aminoacids in NgACSBG sequence predicted to be in
the vicinity of Adenosine monophosphate (AMP).

| | | | | | | |
|---|---|---|---|---|---|---|
| Naga | G341 | AALGLH 373-378 (SEQ ID NO: 53) | D481 | I493 | R496 | N508 |
| ACSBG2 | G365 | TSLGLD 397-402 (SEQ ID NO: 54) | D509 | V521 | H524 | N536 |
| 1PG4 | G387 | DTWWQT 411-416 (SEQ ID NO: 55) | D500 | I512 | R515 | R526 |
| 3EQ6 | G338 | ESYGQT 359-364 (SEQ ID NO: 56) | D446 | F458 | R461 | R472 |

Abbreviations:
NAGA, NgACSBG;
ACSBG2: long-chain-fatty acid-CoA ligase from Gallus gallus;
1PG4, Acetyl-CoA Synthetase from Salmonella enterica;
3EQ6, a human acyl-CoA synthetase medium-chain family member.

The final models were finally energy minimized with the molecular dynamics program CHARMM (Brooks et al., 2009. J Comput Chem 30: 1545-1614) using distance restraints between residues listed in Tables 3 and 4 and AMP or CoA. All missing parameters in the CHARMM force field were obtained from the SwissParam server (Zoete et al., 2011. J Comput Chem 32: 2359-2368). All models were checked for allowed residues in the Ramachandran plot (Ramachandran et al., 1963. J Mol Biol 7: 95-99) and corrected in case of presence of D-amino acids or cis-peptides bonds with Procheck (Laskowski et al., 1993. Journal of Applied Crystallography 26: 283-291). Finally, all models were subject to 1 ns Langevin molecular dynamic (MD) simulation at 300 K to regularize the structures before a final minimization. A summary of model energies and number of misplaced residues is shown in Table 5.

TABLE 5
Evaluation of 3D-models of NgACSBG. Model energies
and number of misplaced residues are indicated.

| NgACSBG | Total energy | Restraint energy | % disallowed |
|---|---|---|---|
| Model | (kcal/mol) | (kcal/mol) | residues |
| M1pg4 | −6346 | 42 | 5.0 |
| M3eq6 | −6346 | 30 | 5.2 |
| Mcons1 | −6485 | 51 | 4.4 |
| Mcons2 | −6003 | 175 | 5.2 |

Three models M1 pg4, M3eq6 and Mcons1 have similar final energy and correctly respect the restraints. Mcons2 shows a larger energy due mainly to the more difficult respect of distance restraints. All of them present in regions distant from the active site, long unstructured loops and some amino acids in disallowed regions of the Ramachandran plot (Ramachandran et al., 1963. J Mol Biol 7: 95-99).

Results

The NgACSBG sequence contains 649 amino acids and is predicted to be soluble with a MW of 71,056 Kda. A 3D structural model was constructed. It seemed essential to propose the positioning of a fatty acid chain in the model, i.e. α-linolenic acid (18:3; O₁₈H₃₀O₂ cis-Δ9, 12, 15), as a substrate for NgACSBG deduced from phenotypic analyses. The search of conserved acyl-CoA synthase motifs was refined via the online servers MOTIF (Kanehisa et al., 2002. Nucleic Acids Res 30: 42-46) and PROSITE (Sigrist et al., 2013. Nucleic Acids Res 41: D344-347), which identified an AMP binding motif, while Superfamily (Wilson et al., 2009. Nucleic Acids Research 37: D380-D386) confirmed that NgACSBG belonged to an Acetyl-CoA synthetase-like family. In terms of secondary structure, not surprisingly, the protein contains both a helices and β-strands: the Porter predicting method (Mirabello and Pollastri, 2013. Bioinformatics 29: 2056-205) found 44% coil, 37% helix and 19% extended conformation. A secondary structure prediction was also made with Predictprotein (Yachdav et al., 2014. Nucleic Acids Res 42: W337-343) (data not shown).

The sequence of NgACSBG was aligned with the sequences of 4 proteins used as templates in our models (see below). The most clearly conserved motif YTSGTTGPPK (residues 216-225) (SEQ ID NO:57), identified as the AMP binding motif, was requalified as playing a fundamental role in ACS catalytic activity (Gulick et al., 2003. Biochemistry 42: 2866-2873; Lopes-Marques et al., 2018. Gene 664: 111-118). The long sequence SITGRIKELIITAGGENIPPVLIE (residues 492-515) (SEQ ID NO:58), or motif II, contains a part of the FACS motif (fatty acyl CoA synthetase signature motif) (Black et al., 1997. J Biol Chem 272: 4896-4903) and supposed to be involved in acyl chain length specificity. In ACSBGs, a so-called motif III (F/YG-SE) (residues 409-413) (SEQ ID NO:59) has been proposed as being involved in AMP binding, whereas Motif IV (LPLSH) (residues 259-263) (SEQ ID NO:60) could be involved in CoA binding (Lopes-Marques et al., 2018. Gene 664: 111-118).

No structure is currently available for an ACSBG in public databases. The sequence was first submitted to Blast (Altschul et al., 1990. Journal of Molecular Biology 215: 403-410) to find sequence alignments with proteins of known 3D structure. The first hit was referenced 1ULT in the Protein Data Bank (PDB, (Burley et al., 2019. Nucleic Acids Research 47: D520-D528)) and corresponds to a long chain fatty acyl-CoA synthetase homodimer from Thermus thermophilus (Hisanaga et al., 2004. J Biol Chem 279: 31717-31726), with 27% sequence identity and 40% homologs. Then the sequence was submitted to several online prediction servers (Robetta (Song et al., 2013. Structure 21: 1735-1742); RaptorX (Kallberg et al., 2012. Nature Protocols 7: 1511-1522); Swiss-Model (Waterhouse et al., 2018. Nucleic Acids Res 46: W296-W303); PhyRe2 (Kelley et al., 2015. Nat Protoc 10: 845-858); I-Tasser (Zhang, 2009. Proteins 77 Suppl 9: 100-113); PsiPred (Buchan et al., 2013. Nucleic Acids Res 41: W349-357) and PS(2) (Huang et al., 2015. Nucleic Acids Res 43: W338-342)). All methods failed to predict the full NgACSBG protein and most of them used 1PG4 (acetyl-CoA synthetase from Salmonella enterica) (Gulick et al., 2003. Biochemistry 42: 2866-2873), and 5MSC (the A domain of carboxylic acid reductase from Nocardia iowensis) (Gahloth et al., 2017. Nat Chem Biol 13: 975-981) as templates (and not 1ULT). Despite its low homology with NgACSBG, another protein was chosen as template because it is another acyl-CoA synthetase with known 3D structure and for which both AMP and CoA are resolved (the first is 1PG4): 3EQ6, a human acyl-CoA synthetase medium-chain family member (Kochan et al., 2009. Journal of Molecular Biology 388: 997-1008).

3D structural homology models were built with Modeller (Sali and Blundell, 1993. J Mol Biol 234: 779-815) for the full length NgACSBG protein, from residue M1 to A649. Modeller being able to use multiple structure alignments, four models were built using different association of template proteins: M1 pg4 from 1PG4 alone, M3eq6 from 3EQ6 alone and two consensus models Mcons1 from 1PG4, 1ULT and 5MSC, and Mcons2 from all four protein templates. The positioning of CoA and AMP in the NgACSBG models was based on their neighboring residues in structure 1PG4 and 3EQ6, also identified in animal ACSBG2 (Steinberg et al., 2000. J Biol Chem 275: 35162-35169) (Tables 3 and 4). The positioning of 18:3 was placed inside the structures, with head in the direction of AMP, respecting its proximity with CoA and profiting of hydrophobic holes seen in the structures. The final models were finally energy minimized with the molecular dynamics program CHARMM (Brooks et al., 2009. J Comput Chem 30: 1545-1614) using distance restraints between residues listed in Tables 3 and 4 and AMP or CoA. All missing parameters in the CHARMM force field were obtained from the SwissParam server (Zoete et al., 2011. J Comput Chem 32: 2359-2368). All models were checked for allowed residues in the Ramachandran plot, corrected in case of presence of D-amino acids or cis-peptides bonds and subjected to 1 ns Langevin molecular dynamic (MD) simulation at 300 K to regularize the structures before a final minimization.

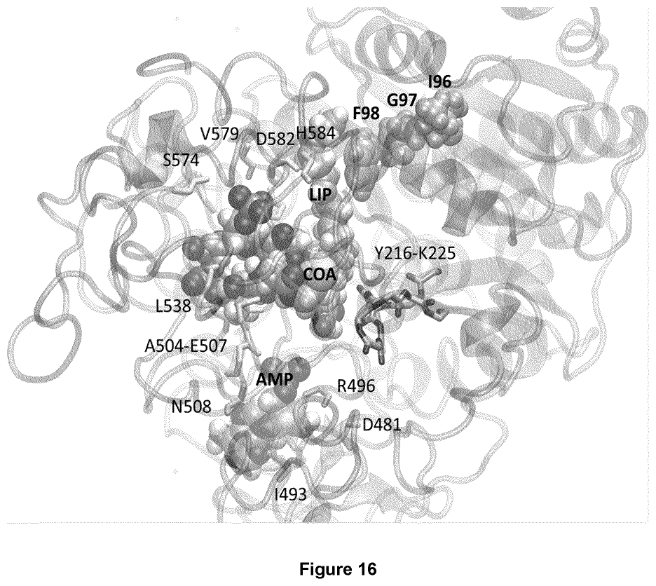

Three models of NgACSBG, M1 pg4, M3eq6 and Mcons1, had similar final energy and correctly respect the restraints. Mcons2 showed a larger energy due mainly to the more difficult respect of distance restraints. From model Mcons1 of NgACSBG (FIG. 16), the proximity of the IGF triad, mutated in this study, to the inserted 18:3 fatty acid was clear. Their relative position is globally the same in all models.

This modelling indicates that the mutation could affect the accessibility of the fatty acid to the reaction site rather than interfering with the reaction itself through proximity with AMP or CoA.

1. A method for the increased production of one or more molecules of interest of the lipid metabolic pathway in a micro-organism, said method comprising:
providing a recombinant micro-organism which has been genetically engineered to express a mutant of an acyl-CoA synthase gene of the Bubblegum type,
culturing said recombinant micro-organism thereby allowing the production of said one or more molecule of interest; and, optionally,
recovering said one or more molecules of interest.

2. The method according to claim 1, wherein the molecules of interest are triacylglycerols, fatty acids, hydrocarbons or fatty alcohols, preferably triacylglycerols.

3. The method according to claim 1, wherein the triacylglycerol content in said recombinant micro-organism is at least 150% of the triacylglycerol content of a corresponding micro-organism which does not express said mutant of an acyl-CoA synthase gene.

4. The method according to claim 1, wherein said micro-organism is a microalga, preferably a microalga selected from the Heterokonta phylum, more preferably a microalga selected from the Bacillariophycea or Eustigmatophyceae, most preferably a microalga selected from the Nannochloropsis genus or Phaeodactylum genus.

5. The method according to claim 1, wherein said acyl-CoA synthase of the Bubblegum type has the amino acid sequence set forth in SEQ ID NO:7 or 8, or a homolog thereof having the Motif II sequence set forth in SEQ ID NO:1, wherein said homolog has preferably a sequence set forth in SEQ ID NO:9-14 or 22-28.

6. The method according to claim 1, wherein said mutant of an acyl-CoA synthase of the Bubblegum-type is a mutant of a Bubblegum-type acyl-CoA synthase from a Heterokonta species, preferably a mutant of a Nannochloropsis or Phaeodactylum Bubblegum-type acyl-CoA synthase, more preferably a mutant of the Bubblegum-type acyl-CoA synthase set forth in SEQ ID NO:7 or 8.

7. The method according to claim 6, wherein said mutant of an acyl-CoA synthase of the Bubblegum-type is a protein comprising 1-5 amino acid substitutions, deletions or additions in a region corresponding to the region between amino acid 83 and 102 of amino acid sequence SEQ ID NO. 7 or in a region corresponding to the region between amino acid 85 and 104 of amino acid sequence SEQ ID NO. 8.

8. The method according to claim 7, wherein said mutant of an acyl-CoA synthase of the Bubblegum-type is a protein comprising one or more amino acid substitutions and/or additions in the region between amino acids 96 and 98 of the amino acid sequence set forth in SEQ ID NO:7 or in a region corresponding to the region between amino acids 96 and 98 of the amino acid sequence set forth in SEQ ID NO:7.

9. The method according to claim 8, wherein said mutant of an acyl-CoA synthase of the Bubblegum type has the amino acid sequence set forth in SEQ ID NO:15, 16 or 44 or has the corresponding mutations of SEQ ID NO:15, 16 or 44 with respect to SEQ ID NO:7.

10. The method according to claim 1, comprising introducing a mutation in an endogenous gene encoding for the acyl-CoA synthase of the Bubblegum-type.

11. A recombinant micro-organism, preferably recombinant microalga comprising a polynucleotide sequence encoding a mutated acyl-CoA synthase protein of the Bubblegum type, wherein said protein comprises 1-5 amino acid substitutions, deletions or additions in a region corresponding to the region between amino acid 83 and 102 of amino acid sequence SEQ ID NO. 7.

12. The recombinant micro-organism according to claim 10, wherein said protein comprises one or more amino acid substitutions and/or additions in amino acids 96-98 of the amino acid sequence SEQ ID NO. 7 or in a region corresponding to amino acids 96-98 of the amino acid sequence SEQ ID NO. 7.

13. The recombinant micro-organism according to claim 10, wherein the mutated acyl-CoA is a mutated Nannochloropsis gaditana bubblegum-type acyl-CoA synthase having an amino acid sequence of SEQ ID NO. 7, or a mutant of the Phaeodactylum tricornutum bubblegum-type acyl-CoA synthase having an amino acid sequence of SEQ ID NO. 8.

14. The recombinant micro-organism according to claim 10, wherein said mutated acyl-CoA synthase protein has the amino acid sequence set forth in SEQ ID NO.15, 16 or 44, or comprises the corresponding mutations of SEQ ID NO. 15, SEQ ID NO. 16 or SEQ ID NO. 44 with respect to the wildtype SEQ ID NO. 7.

15. A vector comprising a polynucleotide encoding a mutated acyl-CoA synthase of the Bubblegum-type wherein said mutated acyl-CoA synthase comprises 1-5 amino acid substitutions, deletions or additions in a region corresponding to the region between amino acid 83 and 102 of amino acid sequence SEQ ID NO. 7.

16. Use of a recombinant micro-organism according to claim 11, for the production of molecules of the lipid metabolic pathway preferably for the production of triacylglycerols, fatty acids, hydrocarbons or fatty alcohols.


SEQUENCE LISTING (60 sequences)

SEQ ID NO: 1 - protein, 35 aa, Artificial Sequence
  sequence of Motif II, a conserved motif in the protein sequence of acyl-CoA synthase (bubblegum type) enzymes
  misc_feature (2)..(5): Xaa can be any naturally occurring amino acid
  misc_feature (9)..(9): Xaa can be any naturally occurring amino acid
  misc_feature (21)..(21): Xaa can be any naturally occurring amino acid
  misc_feature (25)..(25): Xaa can be any naturally occurring amino acid
  misc_feature (28)..(30): Xaa can be any naturally occurring amino acid
  misc_feature (32)..(34): Xaa can be any naturally occurring amino acid
GXXXXTGRXKELIITAGGENXPPVXIEXXXKXXXP

SEQ ID NO: 2 - DNA, 19 bp, Artificial Sequence
  binding sequence of the Tal2Ng-L subunit
tcctgcgcatggtgccatt

SEQ ID NO: 3 - DNA, 19 bp, Artificial Sequence
  binding sequence of the Tal2Ng-R subunit
tggcaatgagccattcggg

SEQ ID NO: 4 - DNA, 9441 bp, Artificial Sequence
  pCT61
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctcagctgctgccccgaccgtatctccaagtcagacatgaaatcttcagttgcgttaaaaactctacgatgctaccagcgttaaataaccttgcccacgcctttaaacgtacccgatcattaacatatcgactggctgccttggctttgcaccagccatcatcagacttaacgatgggtatgttgcttgcctttcctgcttgaagggggtccgactctctgctttctcgatcgcgggtgtgacctctgaattggaatgtaaaaatgtaagaagcgacgtgtccggtaaagaaatgcccaagctccatcaaatctgcgttgtcggcgaccaaaccatgctggctcgtcgacctgccccggatgcaggagcatggcactcggcggcatggcacttgagcctcgcgggaggaatgtgtgtggttgggcgcaggctgtggacggcccccctccagcgaagcggtcgcctccctttccgacgctttgtgcacgttgtctggtgtcctctgtctcacgcacctcttcaccgacgtggtgtccctcttgttgctggtgagggacttggaatgtggtcctggttctatcctgggcgcgtgtgttcctttttttctctaccgttattctctccatttctgatgtctcaccaccatctccctcaccctccaaccgcgtcgttgtgccaaaatcatacagcaggatcgatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaatcgatcttccttaaaaatttaattttcattagttgcagtcactccgctttggtttcacagtcaggaataacactagctcgtcttcaccatggatgccaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgagagctgctgccccgaccgtatctccaagtcagacatgaaatcttcagttgcgttaaaaactctacgatgctaccagcgttaaataaccttgcccacgcctttaaacgtacccgatcattaacatatcgactggctgccttggctttgcaccagccatcatcagacttaacgatgggtatgttgcttgcctttcctgcttgaagggggtccgactctctgctttctcgatcgcgggtgtgacctctgaattggaatgtaaaaatgtaagaagcgacgtgtccggtaaagaaatgcccaagctccatcaaatctgcgttgtcggcgaccaaaccatgctggctcgtcgacctgccccggatgcaggagcatggcactcggcggcatggcacttgagcctcgcgggaggaatgtgtgtggttgggcgcaggctgtggacggcccccctccagcgaagcggtcgcctccctttccgacgctttgtgcacgttgtctggtgtcctctgtctcacgcacctcttcaccgacgtggtgtccctcttgttgctggtgagggacttggaatgtggtcctggttctatcctgggcgcgtgtgttcctttttttctctaccgttattctctccatttctgatgtctcaccaccatctccctcaccctccaaccgcgtcgttgtgccaaaatcatacagcaggaggcctgtcgacggcgcgccatggactacaaggaccacgacggcgactacaaggaccacgacatcgactacaaggacgacgacgacaagatggctcccaagaagaagcgcaaggtcggcatccacggcgtccccgctgctgtctacccctacgacgtccccgactacgctagcggccgcgccaccatgggaaaacctattcctaatcctctgctgggcctggattctaccatggaccctattagaagcagaacaccctctccagccagagaactgctgtctggacctcagcctgatggagtgcagcctacagccgatagaggagtgtctcctcctgccggaggacctctggatggactgcctgcccggagaacaatgagcagaacaagactgccttctcctccagccccatctcctgccttttctgccgattcttttagcgacctgctgagacagtttgaccccagcctgtttaataccagcctgttcgatagcctgcctccttttggagcccaccacacagaggccgccacaggcgaatgggatgaagtgcagtctggactgagagccgccgatgcccctcctcctacaatgagagtggccgtgacagccgccagacctcctagagccaaacctgcccctagaaggagagccgcccagccttctgatgcctctcctgccgcccaggtggacctgagaacactgggatattctcagcagcagcaggagaagatcaagcccaaggtgaggtctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacagtggtctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacgggctgacccccgaacaggtggtggccattgccagccacgacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggcctcactcccgaacaggtggtggccattgccagcaacaacggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacggactgacccccgaacaggtggtggccattgccagcaacaacggcggcaagcaggccctggaaaccgtgcagagactgctgcccgtgctgtgccaggcccatggcctgacacctgaacaggtggtggctatcgcctctcacgacggaggaaaacaagcactcgagacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacatcggcggcaagcaggccctggaaaccgtgcagagactgctgcccgtgctgtgccaggcccatggcctgacacctgaacaggtggtggctatcgcctctaacggcggaggaaaacaggctctggaaacagtgcagcggctgctgcctgtgctgtgtcaggctcacggcttgactccacagcaggtcgtggcaattgctagcaacggcggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaaatagaagaatccccgagcggacctctcacagagtggccgatcacgcccaggtggtgagagtgctgggattttttcagtgtcactctcaccctgcccaggcctttgatgatgccatgacacagtttggcatgagcagacacggactgctgcagctgtttagaagagtgggagtgacagaactggaggccagaagcggaacactgcctccagcctctcagagatgggatagaattctgcaggccagcggaatgaagagagccaaaccttctcctaccagcacccagacacctgatcaggccagcctgcacgcctttgccgattctctggaacgggatctggacgccccttctcctatgcacgaaggagatcagacaagagccagcagcagaaagagaagcaggtctgatagagccgtgacaggaccttctgcccagcagtcttttgaagtgagagtgcctgaacagagagatgccctgcatctgcctctgctgtcttggggagtgaaaagacctagaacaagaatcggaggactgctggaccctggaacacctatggatgccgatctggtggcctcttctacagtggtgtgggaacaggatgccgatccttttgccggaacagccgatgatttccctgcctttaatgaggaagaactggcctggctgatggaactgctgcctcagggatcccagctggtgaaatctgagctggaggagaagaagtctgagctgagacacaagctgaagtacgtgcctcacgagtacatcgagctgatcgagatcgccagaaatagcacccaggatagaatcctggagatgaaggtgatggagttcttcatgaaggtgtacggctacagaggaaagcacctgggaggaagcagaaaacctgacggagccatttatacagtgggcagccctatcgattatggcgtgatcgtggatacaaaggcctacagcggaggctacaatctgcctattggacaggccgatgagatgcagagatacgtggaggagaaccagaccaggaacaagcacatcaaccctaacgagtggtggaaggtgtacccttctagcgtgaccgagttcaagttcctgtttgtgagcggccacttcaagggcaattataaggcccagctgaccaggctgaaccacatcacaaattgtaatggcgccgtgctgtctgtggaggaactgctgattggaggagagatgattaaggccggaacactgacactggaggaggtgagaagaaagttcaacaacggcgagatcaacttctgaaagctttctagacttccttaaaaatttaattttcattagttgcagtcactccgctttggtttcacagtcaggaataacactagctcgtcttcaccatggatgccaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgagggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 5 - DNA, 9438 bp, Artificial Sequence
  pCT62
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctcagctgctgccccgaccgtatctccaagtcagacatgaaatcttcagttgcgttaaaaactctacgatgctaccagcgttaaataaccttgcccacgcctttaaacgtacccgatcattaacatatcgactggctgccttggctttgcaccagccatcatcagacttaacgatgggtatgttgcttgcctttcctgcttgaagggggtccgactctctgctttctcgatcgcgggtgtgacctctgaattggaatgtaaaaatgtaagaagcgacgtgtccggtaaagaaatgcccaagctccatcaaatctgcgttgtcggcgaccaaaccatgctggctcgtcgacctgccccggatgcaggagcatggcactcggcggcatggcacttgagcctcgcgggaggaatgtgtgtggttgggcgcaggctgtggacggcccccctccagcgaagcggtcgcctccctttccgacgctttgtgcacgttgtctggtgtcctctgtctcacgcacctcttcaccgacgtggtgtccctcttgttgctggtgagggacttggaatgtggtcctggttctatcctgggcgcgtgtgttcctttttttctctaccgttattctctccatttctgatgtctcaccaccatctccctcaccctccaaccgcgtcgttgtgccaaaatcatacagcaggatcgatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaatcgatcttccttaaaaatttaattttcattagttgcagtcactccgctttggtttcacagtcaggaataacactagctcgtcttcaccatggatgccaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgagagctgctgccccgaccgtatctccaagtcagacatgaaatcttcagttgcgttaaaaactctacgatgctaccagcgttaaataaccttgcccacgcctttaaacgtacccgatcattaacatatcgactggctgccttggctttgcaccagccatcatcagacttaacgatgggtatgttgcttgcctttcctgcttgaagggggtccgactctctgctttctcgatcgcgggtgtgacctctgaattggaatgtaaaaatgtaagaagcgacgtgtccggtaaagaaatgcccaagctccatcaaatctgcgttgtcggcgaccaaaccatgctggctcgtcgacctgccccggatgcaggagcatggcactcggcggcatggcacttgagcctcgcgggaggaatgtgtgtggttgggcgcaggctgtggacggcccccctccagcgaagcggtcgcctccctttccgacgctttgtgcacgttgtctggtgtcctctgtctcacgcacctcttcaccgacgtggtgtccctcttgttgctggtgagggacttggaatgtggtcctggttctatcctgggcgcgtgtgttcctttttttctctaccgttattctctccatttctgatgtctcaccaccatctccctcaccctccaaccgcgtcgttgtgccaaaatcatacagcaggaggcctgtcgacggcgcgccatggactacaaggaccacgacggcgactacaaggaccacgacatcgactacaaggacgacgacgacaagatggctcccaagaagaagcgcaaggtcggcatccacggcgtccccgctgctgtccaccaccaccaccaccaccaccacagcggccgcgccaccatgggaaaacctattcctaatcctctgctgggcctggattctaccatggaccctattagaagcagaacaccctctccagccagagaactgctgtctggacctcagcctgatggagtgcagcctacagccgatagaggagtgtctcctcctgccggaggacctctggatggactgcctgcccggagaacaatgagcagaacaagactgccttctcctccagccccatctcctgccttttctgccgattcttttagcgacctgctgagacagtttgaccccagcctgtttaataccagcctgttcgatagcctgcctccttttggagcccaccacacagaggccgccacaggcgaatgggatgaagtgcagtctggactgagagccgccgatgcccctcctcctacaatgagagtggccgtgacagccgccagacctcctagagccaaacctgcccctagaaggagagccgcccagccttctgatgcctctcctgccgcccaggtggacctgagaacactgggatattctcagcagcagcaggagaagatcaagcccaaggtgaggtctacagtggcccagcaccacgaagccctggtgggacacggatttacacacgcccacattgtggccctgtctcagcaccctgccgccctgggaacagtggccgtgaaatatcaggatatgattgccgccctgcctgaggccacacacgaagccattgtgggagtgggaaaacagtggtctggagccagagccctggaagccctgctgacagtggccggagaactgagaggacctcctctgcagctggatacaggacagctgctgaagattgccaaaaggggcggagtgaccgcggtggaagccgtgcacgcctggagaaatgccctgacaggagcccctctgaacctgacccccgaacaggtggtggccattgccagcaacaacggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacggactgacccccgaacaggtggtggccattgccagcaacatcggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaacggcggggggaaacaggccctggaaactgtgcagcgcctgctgccagtgctgtgccaggctcacgggctgacccccgaacaggtggtggccattgccagcaacaacggcggcaagcaggccctggaaaccgtgcagagactgctgcccgtgctgtgccaggcccatggcctgacacctgaacaggtggtggctatcgcctctaatatcggaggaaaacaggctctggaaacagtgcagcggctgctgcctgtgctgtgtcaggctcacggcttgactccagaacaggtggtggctattgcttccaacaacggggggaaacaggccctggaaactgtgcagcgcctgctgccagtgctgtgccaggctcacggcctcactcccgaacaggtggtggccattgccagccacgacggcggcaagcaggccctggaaaccgtgcagagactgctgcccgtgctgtgccaggcccatggcctgacacctgaacaggtggtggctatcgcctctcacgacggaggaaaacaggctctggaaacagtgcagcggctgctgcctgtgctgtgtcaggctcacggcttgactccagaacaggtggtggctattgcttccaatattggggggaaacaggccctggaaactgtgcagcgcctgctgccagtgctgtgccaggctcacggactgacccccgaacaggtggtggccattgccagcaacggcggcggcaagcaggccctggaaaccgtgcagagactgctgcccgtgctgtgccaggcccatggcctgacacctgaacaggtggtggctatcgcctctaacggcggaggaaaacaagcactcgagacagtgcagcggctgctgcctgtgctgtgtcaggctcacggcttgactccagaacaggtggtggctattgcttcccacgacggggggaaacaggccctggaaactgtgcagcgcctgctgccagtgctgtgccaggctcacgggctgacccccgaacaggtggtggccattgccagcaacaacggcggcaagcaggccctggaaaccgtgcagagactgctgcccgtgctgtgccaggcccatggcctgacacctgaacaggtggtggctatcgcctctaacaacggaggaaaacaggctctggaaacagtgcagcggctgctgcctgtgctgtgtcaggctcacggcttgactccacagcaggtcgtggcaattgctagcaacaacggcggacggcccgccctggagagcattgtggcccagctgtctagacctgatcctgccctggccgccctgacaaatgatcacctggtggccctggcctgtctgggaggcagacctgccctggatgccgtgaaaaaaggactgcctcacgcccctgccctgatcaagagaacaaatagaagaatccccgagcggacctctcacagagtggccgatcacgcccaggtggtgagagtgctgggattttttcagtgtcactctcaccctgcccaggcctttgatgatgccatgacacagtttggcatgagcagacacggactgctgcagctgtttagaagagtgggagtgacagaactggaggccagaagcggaacactgcctccagcctctcagagatgggatagaattctgcaggccagcggaatgaagagagccaaaccttctcctaccagcacccagacacctgatcaggccagcctgcacgcctttgccgattctctggaacgggatctggacgccccttctcctatgcacgaaggagatcagacaagagccagcagcagaaagagaagcaggtctgatagagccgtgacaggaccttctgcccagcagtcttttgaagtgagagtgcctgaacagagagatgccctgcatctgcctctgctgtcttggggagtgaaaagacctagaacaagaatcggaggactgctggaccctggaacacctatggatgccgatctggtggcctcttctacagtggtgtgggaacaggatgccgatccttttgccggaacagccgatgatttccctgcctttaatgaggaagaactggcctggctgatggaactgctgcctcagggatcccagctggtgaaatctgagctggaggagaagaagtctgagctgagacacaagctgaagtacgtgcctcacgagtacatcgagctgatcgagatcgccagaaatagcacccaggatagaatcctggagatgaaggtgatggagttcttcatgaaggtgtacggctacagaggaaagcacctgggaggaagcagaaaacctgacggagccatttatacagtgggcagccctatcgattatggcgtgatcgtggatacaaaggcctacagcggaggctacaatctgcctattggacaggccgatgagatgcagagatacgtggaggagaaccagaccaggaacaagcacatcaaccctaacgagtggtggaaggtgtacccttctagcgtgaccgagttcaagttcctgtttgtgagcggccacttcaagggcaattataaggcccagctgaccaggctgaaccacatcacaaattgtaatggcgccgtgctgtctgtggaggaactgctgattggaggagagatgattaaggccggaacactgacactggaggaggtgagaagaaagttcaacaacggcgagatcaacttctgaaagctttctagacttccttaaaaatttaattttcattagttgcagtcactccgctttggtttcacagtcaggaataacactagctcgtcttcaccatggatgccaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgagggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 6 - DNA, 20 bp, Artificial Sequence
  reverse primer for TALEN left subunit
gcaggtcgctaaaagaatcg

SEQ ID NO: 7 - protein, 649 aa, Nannochloropsis gaditana
MPKYTTTVASGEVDLRIEKEGPGSWAPKTVFQVFEETVKKYGDSPALHYKKVPHGGSLATTEWSSYTWREYYDLTLEFCKSLLSLGFPAHGAINLIGFNSPEWLIANCGAIAAGGVGVGIYTSNGVDACKYITEHSEAEVVVVENAKQLEKYLKIAKELPRLKALVIYSGTAEGYKCDVPIYSWKDFMALGSGVKDEAVRARIEAQRPGHCCTLIYTSGTTGPPKAVMISHDNLTWTVKNFVASLPFTLTCEDRSVSYLPLSHVAAQMLDIHCPIATGAKIYFAQPDALRGSLPVTLKDVCPTYFFGVPRVWEKIYEKMQEVARSTTGVKRALAQWAKAKGLEKNRRQQYGCGGGAPVGFGCAHALVLSKVKAALGLHQTKMCITSAAPIAVEILEYFASLDIPVLELFGQSECTGPHTSNFSYAWKIGSIGRDIPGVKTKQHANMSEFCMYGRHIMMGYMKMEDKTQEAVDNEGWLHSGDVAQVDADGFWSITGRIKELIITAGGENIPPVLIENEIMSALPAVANCMVVGDKKKFLTVLLTMKAKLDDQGNPTKELNKEALDIGKEIGSNASTTEQVASDPHWKKYFDEGLKKANSTATSNAQFVQKWSVLPLDFSEKGGELTPTLKLKRSVVAEKYADVIADMYKA

SEQ ID NO: 8 - protein, 663 aa, Phaeodactylum tricornutum
MSSLPLSTSEASQEVAIRMGNEGIASLPPKTVMQNFDAQVTKNGNRTALYHKRPKQGQDVADVAWTKYSWKEYRQQVDSFGKALLKVGFARFDTINIIGFNAPEWFFANFGAIAAGGIPAGVYTTNNPEACAYVAEHSEAKVVVCEGVKQLEKYYEISRNLPNLTALVMYGTDSIPEDVKSKCSVPVYTFEGFLDLGKDVSDTDLKARTDSWKAGETCTLIYTSGTTGPPKAVMITNDNITWTIETLMGRTRKGTLDHNDVMISYLPLSHIAAQMLDMHNPMATGTQLYFADANALKGSLGQTLKEVRPTVFFGVPRVWEKIYDKLQEVARSSTGIKKMLSTWAKGKAAAHWESLEYGSKSGSPFMLFLAKKLLHKAHLALGFDRCIQFYVSAAPIEVKILKYFMSLDIPIMELFGQSECTGPHAVNGYDAFKVGTVGRPLIGTETKIDEATGELCYRGRHIFAGYMGMPDKTTETIDSQGWLHSGDIVAIDSDHHPEIKKPSGFISITGRIKELIITAGGENVAPVLIEDELKAAMPALANAMVIGDKRKFLSVLLCLQVEPDIEGNPTNKLTGNALEAAKTIGSKAKTTNEARDDPKWKEYFDKGLEVANKKAISRASRVGKWALLSTDFSEVGGELTPTMKLKRNVTAEKFADTIDAMYA

SEQ ID NO: 9 - protein, 648 aa, Nannochloropsis oculata
MPAYTTTSASGEVDLRMEKEGPGAWEPRTVYQVFEETVQRYGDRPALHFKKVPHGGSPETTGWSVYAWREYYDLTLTFAKSLLALDFPAHGAINIIGFNSPEWLIANCGAIAAGGVGVGIYTSNNAEACNYISEHSEAEVVVVENAKQLEKYVKIAKNLPRLKALVVYDGTGEGFTCDTPIYSWKAFMALGKDKSEAAVRARIEAQRPGHCCTLIYTSGTTGPPKAVMISHDNLTWTVKNFVAALPFTLTCEDRSVSYLPLPHVAAQMLDVHCPIASGAKIYFAQADALRGSLPNTLKDACPTYFFGVPRVWEKIYEKMQEVARSTTGVKRALAQWAKAKGLEKNRRQQYGGGGGAPVGFGCAYALVLSKVKAALGLHQTKICITSAAPISVEVLEYFASLDIPVLELFGQSECTGPHTSNFSYAWKIGSIGRDIPGVKTKQEAAAKEFCMFGRHIMMGYMKMEEKTKEAVDEEGWLHSGDVADVDADGFWTITGRIKELIITAGGENIPPVLIETEVKAALHAVANCMVVGDKKKFLTVLLTMKTKLDEQGNPTNALNREALDIGKELGSEATTTEQVGKDPAWKKYFDEGLKKANAAATFNAQFVQKWAVLPLDFSEKGGELTPTLKLKRSVVAEKYADVTANLYK

SEQ ID NO: 10 - protein, 648 aa, Nannochloropsis oceanica
MPAYTTTSASGEVDLRMEKEGPGAWEPRTVYQVFEETVQRYGDRPALHFKKVPHGGSPETTEWSVYTWREYYDLTLTFAKSLLALDFPAHGAINIIGFNSPEWLIANCGAIAAGGVGVGIYTSNNAEACNYISEHSEAEVVVVENAKQLEKYVKIAKNLPRLKALVVYDGTGEGFTCDTPIYSWKAFMALGKDKSEAAVRARIEAQRPGHCCTLIYTSGTTGPPKAVMISHDNLTWTVKNFVAALPFTLTCEDRSVSYLPLSHVAAQMLDVHCPIASGAKIYFAQADALRGSLPNTLKDVCPTYFFGVPRVWEKIYEKMQEVARSTTGVKRALAQWAKAKGLEKNRRQQYGGGGGAPVGFGCAYALVLSKVKAALGLHQTKICITSAAPISVEVLEYFASLDIPVLELFGQSECTGPHTSNFSYAWKIGSIGRDIPGVKTKQEAAAKEFCMFGRHIMMGYMKMEEKTKEAVDEEGWLHSGDVADVDADGFWTITGRIKELIITAGGENIPPVLIETEVKAALHAVANCMVVGDKKKFLTVLLTMKTKLDEQGNPTNALNREALDIGKELGSEATTTEQVGKDPAWKKYFDEGLKKANAAATSNAQFVQKWAVLPLDFSEKGGELTPTLKLKRSVVAEKYADVIANLYK

SEQ ID NO: 11 - protein, 645 aa, Pythium insidiosum
  misc_feature (104)..(104): Xaa can be any naturally occurring amino acid
MSNFVTWKIDEEVKIRMGDSAIESLPPKTVADVFHATLRKHGAANALHVKRNGEWQTLTFQQYYDKCAQFAKALIHLGLPRFSAVSIIGFNSPEWAITEIGTIXAGGVAAGIYTTNSPSACEYIAKHSDSEVVVCDGAKQLEKFVAIEKNLPKLKALVIYNEPVPEGLQTKVPVYQFEDFLKLGSEVSDATLKQRMEEPKAGNCCTLIYTSGTTGDPKAVMISHDNVTWTIDTVIRMLEKQFSITVDNSHRLVSYLPLSHIAGQLIDVMLPIFSGTQLYFAQPDALKGSLGVTLKEVRPTLFFGVPRVWEKIAEKMWSIAAQTTGIKKRIATWAKNKAAIKTQLAQFGESGGAPCGFGVAHALVLGRVKEALGLDQCHACFSGAAPISREVVEYFGSLDLPIYEFFGQSEATGPQTCSMRGMWRIGTCGSTIVGAHTKVVPETEELIFSGRNVMMGYLKSEAQTHETIDAEGWLHSGDCGKLDADGFMTITGRIKELIITAGGENIPPILIEDAIKEELPILSNAMLIGDKRKFLSALFTFRVEVDSEGLPTNKLDAKALSICKEIGSSATTVAEAKADEKIQKYLTEGVKRANAKATSRAQNVAKFEVLDVDFSIAGNELTPTLKLKRKIVLEKYAETIEKIYA

SEQ ID NO: 12 - protein, 646 aa, Phytophthora parasitica
MASEYTTWKIDEEVKIRMTESGAGARPAITAAQAFQNTLAKYGSANALHYKKDGEWASYSFQQYYDKCCQFAKSLLHVGLERYQGVSIIGFNSPEWAIADVGAIFAGGVAAGIYTTNNPKACEFIARHSDSGVVVCDGMKQLEKFLAIAKDLPKLKALVMWNDVVPEGIQSPVPVYSFEDFMELGKDVKDETLKEIMDSQKPGNCCTLIYTSGTTGDPKAVMISHDNVVWTILSVVGMIKRNFNHQMHNGDRLVSYLPMSHVAAQLIDIWLPICGGLQIYFAQPDALKGSLGVTLKEVRPTFFFGVPRVWEKIAEKMWSIAAQTTGIKKRIAAWAKDKAAQKTALAQYGNSGGAPCGFGVANAVVLMRVKEALGLDQCIASFSGAAPISREVVEYFGSLDLPVYEFFGQSEACGPQTCSMNGNWKISTCGRTIDGSETKVVPETQELIFGGRNIMMGYLKGEQQTKDTIDEDGWLHSGDCGKIDEDGFMTITGRIKELLITAGGENVPPVIIEDTIKEELPLLSNVMVIGDRRKFLSALFTLRVTVDKEGQPTDTLDEKALHVMKEIGSSATTVAEARADEKVKAYLDEGLKRANARATSRAQNIAKYTVLDHDFSINGNELTPTLKLKRKVVYEKYESVIESLYA

SEQ ID NO: 13 - protein, 660 aa, Ectocarpus siliculosus
MSGTVWATERDADLPVKIGTAGAAARPAKTVFEVFQKAVENHGENPALMFKDTSGGMSADDAPWQTLTWAQYYMETLRFGKSLLSLDFAPHRCVNIIGFNSTEWFIANMGAIAAGGIAAGIYTSNLPEACQYITNHSEAEVVVVENEAQLAKFVKLAATLTSVKAIVMYRGNVPEETECGFPVYTWKQFMELGSKIPDTKIEERIEAQRPGQCCSLIYTSGTTGQPKAVMISHDNMTWTAETLCAPFNWSHAERGVSYLPLSHVAGQLLDMHAPMAVASSVWFAQPDALKGTLVKTLREVRPTTFVGVPRVWEKMYEKMQEIGRSIHGAKAALSAWSKGRGLANARRKEYSGGGGTPWGFKIANALVFSKVKEKLGLDKANLCFTTAAPIAQETLEYFASLNIHIMEVFGQSECTGPMAVSTKEAWKMGSCGRPMLGTESKLEPGTGELCYRGRNIFMGYMHNPEKTAETIDEDGWMHSGDVATFDEDGDPRIEGPSGFVRITGRIKELIITAGGENIPPVLIESEFKAELPCIANCMVIGDKRKFLTILIALRSEKREDENGGSSTVRLVGDSLTACEKIGSTATTIEEAKADTKWTEYINKAMKACNARTTSNAQRVAKWAFLPHDFSVEGGELTETLKLRRGPTSEKYSDIIEGLYA

SEQ ID NO: 14 - protein, 674 aa, Fistulifera solaris
MSDLPLYTSDIRQEVRIRIGPGQASIPPMRVFDAFDRVVQTYGNEEALFQKRPRKGVPLTETPWTSWTWKQYRDQVNAFGKALLSIGFERFDTVNIIGFNSPEWFICNFGAMAAGGVASGIYTTNGPEACKYITDHSRARVVVVDSVSQLEKYDAISAGLPHLKVIVLYGSDSINRNTIGSVRIYHFDQFIQLGMEISDDRLKHRTGSWKPGETCELVYTSGTTGPPKSVMITHDNITWTARTMLKTARKGYLNKDDKLVSYLPLSHIAAQALDMFQPLFSGCKVYFAQPDALKGTLVDTLREVRPTVFFGVPRVWEKIYDRMQEVAKTVTGVTKIVSQWAKEEAAAYWRAASYEGQKQCSIIREFPLGYYSAQLLLRSVHQKLGLDQCNEFYVSAAPIDTKILQYFQSVDIPILEVFGQSESTGPHLSNQYSAFKVGTVGRPMLGTDTKIDQDTSELRLRGRNIFAGYMGMPQLTEEAFDDEGYLRTGDIATVDDDVHPSIPGQSGFYTITGRIKELIITAGGENIPPVLIETEIKHAMPAVSNVMVIGDKRKFLTVLISLHVELSSEGVPSNRLAGISLDTCKDIGSLAETTEQARDDPKWRLYFDNGLKHANEKATSQAQRVGKWFLIETDFSEKGGELTPTMKLKRSVAAEKYKDAIESMYASVIASPGN

SEQ ID NO: 15 - protein, 649 aa, Artificial Sequence
  NgACSBG#5
MPKYTTTVASGEVDLRIEKEGPGSWAPKTVFQVFEETVKKYGDSPALHYKKVPHGGSLATTEWSSYTWREYYDLTLEFCKSLLSLGFPAHGAINLFRLNSPEWLIANCGAIAAGGVGVGIYTSNGVDACKYITEHSEAEVVVVENAKQLEKYLKIAKELPRLKALVIYSGTAEGYKCDVPIYSWKDFMALGSGVKDEAVRARIEAQRPGHCCTLIYTSGTTGPPKAVMISHDNLTWTVKNFVASLPFTLTCEDRSVSYLPLSHVAAQMLDIHCPIATGAKIYFAQPDALRGSLPVTLKDVCPTYFFGVPRVWEKIYEKMQEVARSTTGVKRALAQWAKAKGLEKNRRQQYGCGGGAPVGFGCAHALVLSKVKAALGLHQTKMCITSAAPIAVEILEYFASLDIPVLELFGQSECTGPHTSNFSYAWKIGSIGRDIPGVKTKQHANMSEFCMYGRHIMMGYMKMEDKTQEAVDNEGWLHSGDVAQVDADGFWSITGRIKELIITAGGENIPPVLIENEIMSALPAVANCMVVGDKKKFLTVLLTMKAKLDDQGNPTKELNKEALDIGKEIGSNASTTEQVASDPHWKKYFDEGLKKANSTATSNAQFVQKWSVLPLDFSEKGGELTPTLKLKRSVVAEKYADVIADMYKA

SEQ ID NO: 16 - protein, 650 aa, Artificial Sequence
  NgACSBG#40
MPKYTTTVASGEVDLRIEKEGPGSWAPKTVFQVFEETVKKYGDSPALHYKKVPHGGSLATTEWSSYTWREYYDLTLEFCKSLLSLGFPAHGAINLRFFQNSPEWLIANCGAIAAGGVGVGIYTSNGVDACKYITEHSEAEVVVVENAKQLEKYLKIAKELPRLKALVIYSGTAEGYKCDVPIYSWKDFMALGSGVKDEAVRARIEAQRPGHCCTLIYTSGTTGPPKAVMISHDNLTWTVKNFVASLPFTLTCEDRSVSYLPLSHVAAQMLDIHCPIATGAKIYFAQPDALRGSLPVTLKDVCPTYFFGVPRVWEKIYEKMQEVARSTTGVKRALAQWAKAKGLEKNRRQQYGCGGGAPVGFGCAHALVLSKVKAALGLHQTKMCITSAAPIAVEILEYFASLDIPVLELFGQSECTGPHTSNFSYAWKIGSIGRDIPGVKTKQHANMSEFCMYGRHIMMGYMKMEDKTQEAVDNEGWLHSGDVAQVDADGFWSITGRIKELIITAGGENIPPVLIENEIMSALPAVANCMVVGDKKKFLTVLLTMKAKLDDQGNPTKELNKEALDIGKEIGSNASTTEQVASDPHWKKYFDEGLKKANSTATSNAQFVQKWSVLPLDFSEKGGELTPTLKLKRSVVAEKYADVIADMYKA

SEQ ID NO: 17 - protein, 20 aa, Artificial Sequence
  domain of NgACSBG 83-102
LSLGFPAHGAINLIGFNSPE

SEQ ID NO: 18 - protein, 671 aa, Nannochloropsis gaditana
MDRYKWRTLPDVFETVASLAPEAVAVEDMVHTPTAKMTYGELNRQIGALAAFFQHEGLKPGQCVSVFAENSHRWLIADQAILKAGACNAVRGVKAPVDELQYIYQNSESVASVVESVEQIEALMRTNGGLTGRYGPPRFILVLFPGERSGQEIRELANLPPPTQVLTFDEALSASLARPLTFRPVPKDVRSVATLVYTSGTTNKPKGVVLRHSNLLHQVNYNSFTDSPSKEPAYNPVLGDVLVSVLPCWHIFERTAEYWMFSKGIHVVYSNVKNFKADLAKHQPQFIVAVPRLLETIYRGVLQKFATEKGAKKKIIEFFTRVGSAWVKAWRVARGLVLRSRAPNPIERLLALVLALVLSPLAAVGDKLVWSKVRAGLGGRIKVLVAGGSSMPLVLEDFFELLRTPVIVGYGMTETSPVITNRVAEKNLAGSVGRTARDTEVKIVDPESGARLPEGQPGLVLMRGPQMMAGYKSNAEASKAVLDQEGFLDTGDLGRIHPLTKHLIITGRAKDTIVLSNGENVEPQPIEDVVCANSALVDQVMCVGQDEKVLGMLVVPNVRALARAGLVDRGLAERVAELLGGQVLTNGIAGSRAELEEVEASLREKKEVKKALLADIARAMGKSFRETERVGAVEVVLEPFNMANGFLTQTLKVKRNVVSGHYAQEIEQMYR

SEQ ID NO: 19 - protein, 889 aa, Nannochloropsis gaditana
MRPERLALSLIFLVWSVANVSHAFMAPLAVRLRQPLHVEHLLPARHPEPPGGRLDTARPHEVPSGSPSRHCNAGKDSSHDYGTATLSEGRHPELIDTHWTQGNPIQLLRQGHIPPFTWTPPLFSPSNATAKKTQDDKSGKSFVDTAALERAIASPGLRSVPGMWTVLAEHCGAWNMLTDPSHSVPPPSLAASSSSTPLSLTFAQAHDVIRLAAAGLHALNLRQGDHIAFFSENSARWLLAEQGASLNGCPTAVRGASASVEELQYIYRTADCKAVVLQDLSLLRKLREAGGLSSKHGPPRLVIVLRPPEGMRERQGVGSGGGEGETEGGLGPLSVATLSEEPDCGPTVSLEELLALGARHLPHYLPPDLRPSDLHTLVFTSGTTGRPKGVMLSHANLLHQVRHIDLGRNPLPGDVVLSLLPCWHIFERSSELFALARGASLVYSSVRAFKADLQRYRPHYLIVVPRLLENVHRSIKDKLSAGSPVRRRLVATLSLLSLLYTRALKQALGLVVRRPASHPPTGSRRHLFLASFVSTLRRLAYRPAAALATCFLLPGALLADLLVWKKVRQALGGRQKLVVSGGSALARYLEEFYDAAGIPVVSGYGLTETSPVIAVRRAGRLRNLVDGGVVGFPPKEVELEIRALEDEREGAPEKRRQERTRKEVGSAEGKVGIVFTRGPQVMQGYYKDPSSTRKAVGDDGWLNTGDLGYLNPGTGDLVLTGRAKDLIVLSNGENVEPQPIEDAVVERCATVEQCLVLGHDKRQLSALVVVSPQGLAARGLLPPVEAERLDALVGPSAVLLGCQGDKTALDMEADRLNADESLVRAVQKEIAAALTQDPEEFRAWEQIKNLHLLLAPFNVQSGLLTQTLKVKRVTVQDYYKEAIDRLYAQ

SEQ ID NO: 20 - protein, 689 aa, Nannochloropsis gaditana
MVYVPHRKKFYSIEVPNTETATAGPIRRFPDGEINPQLAYDGCYTIYELFRRSVAIYPHRPFLGHRPIDSEGHAAPYVFESYEEAARRIDNLASALEHEGLCPPTPDGLRLLGLYMKNCPEWIIADQAAFTLHGATVPLYDTLGPDAVGYVIKQTELAAVVCGPQELENLVAAAPECPSLKAAVLTCSVLSDHHRALARKGGLKLYSLMDLEKLGFRYPQPHFPPDPDDVATFSYTSGTTGGPKGALLTHRNFLTVYCAGKALEVENFPDDIHFSYLPLPHVFERVMLLAVLGNGAAMGFWQGSPERLVEDLQALRPTCMPAVPRVLNRMYDKVMAGVRAASPLKQMLFQRAYAAKLRDLDERGELSKGVWDRLVFAKVRRAIGLDRMRVMVTGSAPIAPHVLQFMRVLCACPIHEGYGQTETTAMTSLTFPGDWTTGHVGGVAPVSEVRLVDVPEMGYLHTDRQHGSAGRGGGAFGKAAQPCIGRGEICVRGPHIFKGYYKQPDKTAETIDAQGWLHSGDVGMWTPEGRLVIIDRKKNIFKLAQGEYVAAEKIENVLMQSEMIAQAFVYGDSLKSELVAIVVPDAEAVQDWARQESLPVDHSFAELCRQSLLKEKILDEIEVQSKRAGLHGFEIPKAIYLESEPFTGANGLLTPTQKLKRMQARDRYSTIIQELYATRDLHQGSKARL

SEQ ID NO: 21 - protein, 1307 aa, Nannochloropsis gaditana
MSGTTSNPHAPSSEAAVNSDGWKMPGPNMDRLATLLAHDRELQDAMPDPQVQARIDKTATTIELVQQVCEGYAEREALKWCVNGAPTADYTECLTYRDIWKRVRAMATVLSGRGWLLKKDFVAVCGFASPGWVLIDVLALYLGAVLVPLPLNVPYEDLRSMLEESEARVVFCAAEEAHTLVMNVLAKDGACPGVETVVVMDYGEESNGQAMVQALRPVLPATVLRVLTLGELLTDVSQGIVDSTERYGTTAAASSCGFLPPAVPGSQGWSDESNPLLGLMYTSGSTGRPKGAMYTEKLMRALWHTGFSWGKGEVPVISLGFLPLNHIVGRVTIYQALSKGGRVYFVRRSDMSTLFEDFATARPTKTMMVPRIANMAYETYQLKKMELLQTVGLSSSSLPTEEQAQGKRLKLDEVERAAREYIRDRVFGGRLLFAIVGTAPSSAAVSALIQEACEMPMVEGYGSTELGGITIENRINVATVVKWKLIDVPELGYTLKDQPCPRGELLVMTTTGIPGYYKHPKATAELIDTEGFFHTGDIMEQRGPEELVWIDRRKNVLKLAQGEYLSISRLEALYAGDPDVKNIFLYGNSLRSYVLAVVVPSEALVAAAKAGLEDGNGGNGLKGSARDELAGQGEVEEEAVLARLKPLLRQRLDSMAKEAGMASYEVPRDFIVQLAGFTRENKLVTDSNKLARARLKETFGPRLEAMYDAVEQRKEKRLEHLHSDPQASTTDMVRAVLEMTLGLGEEVGLDATFAQLGGDSLSAVRVTEHLKRLCGVGVPVAELLNPAMTLSSLIQHLDLKLKRGQVVGAEDMEETGEEEIYRRVHGLGDDDAVIFAQDLTIGKFIPTAVLHPPEAKVATDWAAGAEKGFEKREGRVRGVLLTGANGFLGRFLLLELLQKLCAQTDYKLGGTGRVYCLIRGRDEAAAKCRLWEAVGASLLQAHEDRIVVLAGDVSRPHLGLSGDTYANLLENVDLVLHAGALVNHNLSYRSLFGPNVVGTANIIKFCLARPSHPKPLHYVSTIAVTMGEGGPGREVHEGHLGYCWGNQRFVRKGTYAEGYGASKWAAEVLLQNVHKETGLPVVAYRCSMILPHSSLPGQINVADVFTRLLAGVIYTGVAPASFTDGHGPAPAYDGSPVDFVAGAIVAIVWKGFHRSTGQVDVDAGQEIEASGCRLFHVINPHVQKGPSLDDMIDWVQSAGYDVERITPYRTWLETFESKLQALPSEKRNQSPLPVLKHWQDPSPSRIHAQEISVVDASRFRAAVKELTRWGDVPCLCEGYLLTCLYQMSKVGLISATPAVMLAKLAEK

SEQ ID NO: 22 - protein, 737 aa, Gallus gallus
MRWMMLCESEARSTLADPVPMAYLSVDAQGSSEVSLDDITINSSAGTVEVCSMKPADDPKTERSQMNKTGLASSSRPASNVWTTQQDGEVKLRMDEEGMGSEAPKTVHEVFQEAVSKYGDYYALASKKNGQWVKLTYKMYYDKCWKAAKSFLKLGLERFHGVCILGFNSPEWFIADIGAIFAGGLAVGIYTTNSPEACHYVAENCSANILVVENHTQLQKILEIEHKLPHMKAIIQYGEELKEKRPNLYSWREFLDLGEDVPDSQLREIIESQKPNQCCTLIYTSGTTGQPKGVMLSHDNLTWTSIAAGRSLMLLEATEKQELVVSYLPLSHVAAQMIDIWLPVTFGGQVFFAQPDALKGTLVDTLREVRPTAFLGVPRVWEKIEEKMKSVGAKSSTLRRKVASWAKGVGLQTNLKWMNGHSEVPMNFRLARQLVYKKVRKAIGLDRCTKCFTGAAPISRETLEFFLSLNIPVFELYGMSESSGPHTVSIPQAFRLTSCGKEMAGCRTLIHKPDADGIGEICFAGRHIFMGYLNMEEKTKEAIDKDGWLHSGDLGKCDKDGFIYITGRIKELIITAGGENVPPVPIEDAVKEACPIISNAMLVGDKAKFLAMLLTLKCIINTESGEPGDDLTAEAIEYCQKLGSKATKVSEIISSKDKAVYAAIQAAVSEVNKRAVSNAQKIQKWVVLEKDFSVGGGELGPTMKLKRPVVAQKYKDLIDEFYADANTPTTTENALRQ

SEQ ID NO: 23 - protein, 688 aa, Gallus gallus
MPSSGETLKKNLQDDNGRTGDGRPVCRDLLAHFEETQDEDQEQTESLWTSFADGRVRLRIDNSCPQPPITVHQMFKESLEKYGSLNALASRKNGKWEKITFSEYYCLSRKAAKSFLKLGLERFHSVAILGFNSPEWFISAVGAVFAGGIVTGIYTTNSPEACHYIAYDSKTNIMVVENRKQLDKIMQIWNRLPHLKAVVLYKDSILERHPNLYTMEEFLKLGDDISDVTLDDIINSQKPNQCCVLIYTSGTTGKPKGAMLSHDNITWTSAHCSRAGDMQPAEVQQESIVSYLPLSHIAAQIYDLWTGIKWGEQVYFAEPDALKGSLINTLKEVQPTSHMGVPRVWEKIMEKLKDASAQSGFVKKKMLSWAMSVSLERNLNCSSSSDLKQFWTRLADYLVLAKIRSALGFSSCQKHFCGAAPLNTETLYFFLGLNITLYEAYGMSETTGPHCLSGPYNYRQHSCGKPAPGCRVKLVNEDTEGNGEICFWGRTVFMGYLNMEDKTKEAFDEDGWLHSGDLGKLDKDGFLYVTGRIKDLIITAGGENVPPIPIEDAVKKELPIVSNAMVIGDKKKFLSMLLTLKSVLDPDTSDPTDILTEQARDFCQKIGSKATTVSEIVATRDQAIYQAIQEGINKVNMNATNRVHCIQKWIVLPRDFSISGGELGPTMKLKRLTVLQKYKNEVDSFYED

SEQ ID NO: 24 - protein, 666 aa, Homo sapiens
MTGTPKTQEGAKDLEVDMNKTEVTPRLWTTCRDGEVLLRLSKHGPGHETPMTIPEFFRESVNRFGTYPALASKNGKKWEILNFNQYYEACRKAAKSLIKLGLERFHGVGILGFNSAEWFITAVGAILAGGLCVGIYATNSAEVCQYVITHAKVNILLVENDQQLQKILSIPQSSLEPLKAIIQYRLPMKKNNNLYSWDDFMELGRSIPDTQLEQVIESQKANQCAVLIYTSGTTGIPKGVMLSHDNITWIAGAVTKDFKLTDKHETVVSYLPLSHIAAQMMDIWVPIKIGALTYFAQADALKGTLVSTLKEVKPTVFIGVPQIWEKIHEMVKKNSAKSMGLKKKAFVWARNIGFKVNSKKMLGKYNTPVSYRMAKTLVFSKVKTSLGLDHCHSFISGTAPLNQETAEFFLSLDIPIGELYGLSESSGPHTISNQNNYRLLSCGKILTGCKNMLFQQNKDGIGEICLWGRHIFMGYLESETETTEAIDDEGWLHSGDLGQLDGLGFLYVTGHIKEILITAGGENVPPIPVETLVKKKIPIISNAMLVGDKLKFLSMLLTLKCEMNQMSGEPLDKLNFEAINFCRGLGSQASTVTEIVKQQDPLVYKAIQQGINAVNQEAMNNAQRIEKWVILEKDFSIYGGELGPMMKLKRHFVAQKYKKQIDHMYH

SEQ ID NO: 25 - protein, 724 aa, Homo sapiens
MPRNSGAGYGCPHGDPSMLDSRETPQESRQDMIVRTTQEKLKTSSLTDRQPLSKESLNHALELSVPEKVNNAQWDAPEEALWTTRADGRVRLRIDPSCPQLPYTVHRMFYEALDKYGDLIALGFKRQDKWEHISYSQYYLLARRAAKGFLKLGLKQAHSVAILGFNSPEWFFSAVGTVFAGGIVTGIYTTSSPEACQYIAYDCCANVIMVDTQKQLEKILKIWKQLPHLKAVVIYKEPPPNKMANVYTMEEFMELGNEVPEEALDAIIDTQQPNQCCVLVYTSGTTGNPKGVMLSQDNITWTARYGSQAGDIRPAEVQQEVVVSYLPLSHIAAQIYDLWTGIQWGAQVCFAEPDALKGSLVNTLREVEPTSHMGVPRVWEKIMERIQEVAAQSGFIRRKMLLWAMSVTLEQNLTCPGSDLKPFTTRLADYLVLAKVRQALGFAKCQKNFYGAAPMMAETQHFFLGLNIRLYAGYGLSETSGPHFMSSPYNYRLYSSGKLVPGCRVKLVNQDAEGIGEICLWGRTIFMGYLNMEDKTCEAIDEEGWLHTGDAGRLDADGFLYITGRLKELIITAGGENVPPVPIEEAVKMELPIISNAMLIGDQRKFLSMLLTLKCTLDPDTSDQTDNLTEQAMEFCQRVGSRATTVSEIIEKKDEAVYQAIEEGIRRVNMNAAARPYHIQKWAILERDFSISGGELGPTMKLKRLTVLEKYKGIIDSFYQEQKM

SEQ ID NO: 26 - protein, 721 aa, Mus musculus
MPRGSEAGYCCLSRDSNMPDSRDDQQQGASLGTSQDNSQTSSLIDGQTLSKESPSHGLELSAPEKARAASLDGAEEALWTTRADGRVRLRLEPFCTQRPYTVHQMFYEALDKYGNLSALGFKRKDKWERISYYQYYLIARKVAKGFLKLGLERAHSVAILGFNSPEWFFSAVGTVFAGGIVTGIYTTSSPEACQYISHDCRANVIVVDTQKQLEKILKIWKDLPHLKAVVIYQEPPPKKMANVYTMEELIELGQEVPEEALDAIIDTQQPNQCCVLVYTSGTTGNPKGVMLSQDNITWTARYGSQAGDIQPAEVQQEVVVSYLPLSHIAAQIYDLWTGIQWGAQVCFADPDALKGTLVNTLREVEPTSHMGVPRVWEKIMERIQEVAAQSGFIRRKMLLWAMSVTLEQNLTCPSNDLKPFTSRLADYLVLARVRQALGFAKCQKNFYGAAPMTAETQRFFLGLNIRLYAGYGLSESTGPHFMSSPYNYRLYSSGRVVPGCRVKLVNQDADGIGEICLWGRTIFMGYLNMEDKTCEAIDSEGWLHTGDMGRLDADGFLYITGRLKELIITAGGENVPPVPIEEAVKMELPIISSAMLIGDQRKFLSMLLTLKCTLDPETSEPTDSLTEQAVEFCQRVGSKASTVSEIVGQRDEAVYQAIHEGIQRVNANAAARPYHIQKWAILQRDFSISGGELGPTMKLKRLTVLEKYKDIIDSFYQEQKQ

SEQ ID NO: 27 - protein, 666 aa, Drosophila melanogaster
MSTIDALYNRPGPNRLRQADAYRTTNRQDAVKIRMAKDGIGAEEPISVPGLLKRTVNNYGDYPALRTKNGKNGYHTVTYKQYEQKVHQVAKAFIKLGLEEHHSVGVLAFNCAEWFYSAMGAIHARGIIAGIYTTNSADAVQHVLESSHAQIVVVDDAKQMDKIHAIRDKLPKLKAAIQIQEPYSPYLKKEDGYYRWSEIESMNVSDVEDQYMTRLENVAINECCCLVYTSGTVGMPKGVMLSHDNITFDVRGIVKAMDRVVVGAESIVSYLPLSHVAAQTVDIYTCAFVAGCIWFADKDALKGTLVKSLQDARPTRFMGVPRVYEKFQERMVAVASSSGSLKKMLASWAKGITLKHYMVSQGKSSGGFRYKIAKSLIMSKVKQALGFDRVLTLASAAAPMSPETKKYFLSLDLKIVDAFGMSETAGCHTICLPDSVGLNTIGKTLPGCESKFINKDANGHGELCIRGRHVFMGYIDNKEKTEESLDDDCWLHSGDLGFVDDKGYVSLTGRSKEIIITAGGENIPPVHIENTIKKELDAISNAFLVGEQRKYLTVLITLKTEVDKDSGEPLDELSHESSVWVKSLGVEHKTVSDILAAGPCPKVWKSIEDAIKRANKQSISNAQKVQKFTILPHDFSIPTGELGPTLKVKRNVVSKMYADEIEKLYA

SEQ ID NO: 28 - protein, 666 aa, Drosophila melanogaster; identical to SEQ ID NO: 27

SEQ ID NO: 29 - protein, 721 aa, Phaeodactylum tricornutum
MKYLLFLPVKILLIAIDLLLTIVTFGWLGIIRKITSADQLRTVPVADDESHRVDPRFRAQLITTPRPGMETLYDITKVAFERFGSRHCMGTRHFLGWRTPKIKEFGPDTTWKTYAQVGQQANQFGAALRSAGVQPAPYSTTLEQVTTPCRIAIFENTCAEWMIAAMGAFTQSVTVTTVYATLGMDAVAEAVVDNIIPVIVCNRKDVSRLAQKCSSMPSLKYIVYTDDLVAPNETIEMPKAPKGVTIVSFDEFCASGDTKAYPPTPPKADSCAVLMYTSGSTGKPKGVIVTHRQIVGACAAGDVALQVVPGGETYLAYLPLAHILELMAEFLMITMGCTLCYADPKSLSATGAYPIGALEQFSPTIMVAVPKIWDTIKKGIEAKVHQASPVAQMLVETGFAWRSFALKHGFDTPLFKALVFKKFAKVVGGQLRLAVSGGGPLNTEVQDFIRAAFGVTFIQGYGLTETCAGLTIQDFTDTRGNIAGVPIASVEVKLESTPDVGDKAGQSYLSTDRRDVEGNPIFGRGEIVVRGSNITCGYYMMAEKSKDEFKEDGWFHTGDIGQFMADGSIRIVDRKKNLVKLKGGEYIALEKMEMTYGNSKFVDAVQGGICCYGDGDMDRPIAIMQLSQPYTTKWAQEKGLKGDWEELKTSKEVYNAVLDDMKKEHAKSDLSHLEKLVAVALLDSPWTPENGCLTAANKLQRRAVIQQFEKEFEDVKKKGIF

SEQ ID NO: 30 - protein, 757 aa, Phaeodactylum tricornutum
MKCSKIIVRATIEKKDVMTEEDEDILFGDEAYVPVTSVYRPKSRSGSIFPDPSHPPNPLDHVSSDPLIGKLHTMRDTLRNCPQLWTELSRNCGDKRALLDEHMCDDKVDVTFAEMNTIVRKSAAVFQSLGVKPGVNVAILGENSARWLMVDHGIQMAGGASAVRGADAPLDELRYIYEQSDSAGIVVVQGPKLLEKLASDASAKGMHRLGLSNEAYGKVHTVILMHREKKTSEELTKMVDKYGLTVRVFADLLEEVQPVTDEQIPVLGRDDLATIVYTSGTTGRPKGVMLTHGNLIHQIGHRVGTETRYDESEPLPDETMLSLLPVWHITERSFELWMLARGCNVVYSSIRTFKNDLAKHRPEWAVLVPRVLEKIATGVQDKFSSGSLPVKLLSKLFTSTSKAAAKHRKITQGYVVGEQPPGAMEKLASKALLVALSPLNAVGNKIVWSKVQNGFGGRMRTIMSGGSALSGSLEEFYEAAGLTIIVAYGLTECAPLISHRRRDANLVTGGCVGTACIDTELRVVSPESKASTTPRPALPAGEVGVVIARGPQIMKGYYKDVAETKKAIDQFGFFDTGDLGRVNPATGDLILTGRAKDTIVLSNGENVEPGPLEDAILGELSLIEQVMLTGQDGRKLTAIVVLSPSELANEGFLSKDEGALMQKRNEQVNDPKCSAKDCQDNVAALNQGSKILRTNKDLIARLVGDCKKATKRFQKWEQVKDVYVTLEPFAMANGQLTQSYKVKRPFVLERYEDELSK

SEQ ID NO: 31 - protein, 702 aa, Phaeodactylum tricornutum
MTSLATTTTTRLLMRHAGKRSPWTRRTRMADVSTRRWMGSPETRALSSVSVTPLSTQPDVELSVRQREYYTQGWIGSDGLTAFETLHEMQTRSCQVFAEKRLFGTYSDASKAYEWISFAEFDQAVSVCRAVLRDLGVQEHDKVGLISNNRVEWVTIACAAYSLNANVVPMYEAQLPSDWTYILNDSGAKVVFCATTDIYDVVRSQVRINVPTLQANVCLDAPDGEPFAYSTLMHHATADVDGQFVKLPTPDDLANLIYTSGTTGKPKGVELTHENFCSNVKAGCRSLGDVRKLIKDDDVSLAFLPWAHSYGQTAELWSSMAHGSSMGISRGVSHILEDLQLVQPTVLFAVPTLFKKIYDGVHNAMEAAPPLRKKLMKAALSLGRERVAVNEGTRDALGFIERTKLGVLDKLVLGKIRARFGGKLRHAFVAGAACPPEVLSFMDDVGIQVCEGYGLTETSPIITINTPEYRKVGSVGKAIGGVTVHIVDEEGKELPIGEEGEICCTGPNIMRGYYGNAEATAEVITTAPDGKTRMFHTGDLGRMGEDGFLKVTGRLKELYKLENGKYVCPTPIEEGIGMSRFIAQVVLTGANQPFNVALVVPEWPAIRAELGLDDSVSDDELAMNEKVRALVDADIKIKCKNMKKFEVPQNWAFVAPFTAANDMLTPKMSIRRHKVISTYNDIIAAMYDSESIEKEEVQRKAA

SEQ ID NO: 32 - protein, 678 aa, Phaeodactylum tricornutum
MTNQNMRSTVLPQTDRHDRGEIRRSWNPVVNAELGVYGCRTMYEGFRRGAKVNPLGPCMGFRAVSTTGFATPFIYSSYTECLARIDAFAAGLDTLKLIQPNEDGLAVLALYMKNCMEGAIGEHGAFAIGGSSVLMYDTLGPDTVTFILDQTSARSVVCTRAELPKLCESKVSGKCPAFTAAILVDGVTEDAAKMALEAGLEVLSFAKVEAVGAVCIAEKGHHHRPPAPTDVAAFCYTSGTTGNPKGALLTHENIMSAIAGFYGAVGDLEAQPFDRHLSYLPLAHIFERIVSSQMFCAGASVAFFRGDPTLLIEDMQACRPTVMPVAPRVLNKIYDKIQAGISSVGGLKKKLFDAAVAAKAEGLQSGHMKHALYDRLIFNKIKKGLGMDQLRMMVSGSAPLNDTVMTFFRCMLAIPVVEGYGQTEGAAAATIGSSDDMATVGHVGGPVGSVEIVLVDVPEMGYFHTDTLHRGMACQGRGEICVRGPSVFKGYYKDDLNTRETIDNEGWLHSGDVGLWRPDGNLQIIDRKKNIFKLSQGEYVAPEKIENFLISSPLIGQCFVYGDSYQNSLVGLMVPDEEPVRTWAAVNAPELKSASLAEMCNSEALKAAVMADIQRIGVESRLLGFEKPKDILLVSVPFTIENEMMTPTFKLKRQKIRDTYEKEIDLLYAGLPPPLSKL

SEQ ID NO: 33 - protein, 38 aa, Mus musculus
GFLYITGRLKELIITAGGENVPPVPIEEAVKMELPIIS

SEQ ID NO: 34 - protein, 38 aa, Drosophila melanogaster
GYVSLTGRSKEIIITAGGENIPPVHIENTVKKELDAIS

SEQ ID NO: 35 - protein, 38 aa, Drosophila melanogaster
GNLIISGRLKELIITAGGENIPPVHIEELIKKELPCVS

SEQ ID NO: 36 - protein, 38 aa, Nannochloropsis gaditana
GFWSITGRIKELIITAGGENIPPVLIENEIMSALPAVA

SEQ ID NO: 37 - protein, 39 aa, Phaeodactylum tricornutum
GFISITGRIKELIITAGGENVAPPVLIEDEIKAAMPALA

SEQ ID NO: 38 - DNA, 69 bp, Nannochloropsis gaditana
ggatttcctgcgcatggtgccattaattaatcggtttcaactcccccgaatggctcattgccaactgtg

SEQ ID NO: 39 - DNA, 69 bp, Artificial Sequence
  mutated NgACSBG fragment
ggatttcctgcgcatggtgccattaattatttcggttaaactcccccgaatggctcattgccaactgtg

SEQ ID NO: 40 - DNA, 72 bp, Artificial Sequence
  mutated NgACSBG fragment
ggatttcctgcgcatggtgccattaattacggttttttcaaaactcccccgaatggctcattgccaactgtg

SEQ ID NO: 41 - DNA, 17 bp, Artificial Sequence
  Forward primer for TALE-N left subunit
ccccgactacgctagcg

SEQ ID NO: 42 - DNA, 14 bp, Artificial Sequence
  Reverse primer for TALE-N right subunit
cgctaaaagaatcg

SEQ ID NO: 43 - DNA, 18 bp, Artificial Sequence
  Forward primer for TALE-N righ subunit
caccaccaccaccacagc

SEQ ID NO: 44 - protein, 649 aa, Artificial Sequence
  NgACSBG#31
MPKYTTTVASGEVDLRIEKEGPGSWAPKTVFQVFEETVKKYGDSPALHYKKVPHGGSLATTEWSSYTWREYYDLTLEFCKSLLSLGFPAHGAINLRVFNSPEWLIANCGAIAAGGVGVGIYTSNGVDACKYITEHSEAEVVVVENAKQLEKYLKIAKELPRLKALVIYSGTAEGYKCDVPIYSWKDFMALGSGVKDEAVRARIEAQRPGHCCTLIYTSGTTGPPKAVMISHDNLTWTVKNFVASLPFTLTCEDRSVSYLPLSHVAAQMLDIHCPIATGAKIYFAQPDALRGSLPVTLKDVCPTYFFGVPRVWEKIYEKMQEVARSTTGVKRALAQWAKAKGLEKNRRQQYGCGGGAPVGFGCAHALVLSKVKAALGLHQTKMCITSAAPIAVEILEYFASLDIPVLELFGQSECTGPHTSNFSYAWKIGSIGRDIPGVKTKQHANMSEFCMYGRHIMMGYMKMEDKTQEAVDNEGWLHSGDVAQVDADGFWSITGRIKELIITAGGENIPPVLIENEIMSALPAVANCMVVGDKKKFLTVLLTMKAKLDDQGNPTKELNKEALDIGKEIGSNASTTEQVASDPHWKKYFDEGLKKANSTATSNAQFVQKWSVLPLDFSEKGGELTPTLKLKRSVVAEKYADVIADMYKA

SEQ ID NO: 45 - DNA, 66 bp, Artificial Sequence
  fragment of NgACSBG#31
ggatttcctgcatggtgccatttaattacgggtttttaactccccgaatggctattgccaactgtg

SEQ ID NO: 46 - DNA, 66 bp, Nannochloropsis gaditana
ggatttcctgcatggtgccattaatttaatcggtttcaactccccgaatggctattgccaactgtg

SEQ ID NO: 47 - DNA, 66 bp, Artificial Sequence
  fragment of NgACSBG#5
ggatttcctgcatggtgccattaatttatttcggttaaactccccgaatggctattgccaactgtg

SEQ ID NO: 48 - DNA, 69 bp, Artificial Sequence
  fragment of NgACSBG#40
ggatttcctgcatggtgccattaatttacggttttttcaaaactccccgaatggctattgccaactgtg

SEQ ID NO: 49 - protein, 4 aa, Nannochloropsis gaditana
AGGE

SEQ ID NO: 50 - protein, 4 aa, Gallus gallus
AGGE

SEQ ID NO: 51 - protein, 4 aa, Salmonella enterica
VSGH

SEQ ID NO: 52 - protein, 4 aa, Homo sapiens
SSGY

SEQ ID NO: 53 - protein, 6 aa, Nannochloropsis gaditana
AALGLH

SEQ ID NO: 54 - protein, 6 aa, Gallus gallus
TSLGLD

SEQ ID NO: 55 - protein, 6 aa, Salmonella enterica
DTWWQT

SEQ ID NO: 56 - protein, 6 aa, Homo sapiens
ESYGQT

SEQ ID NO: 57 - protein, 10 aa, Nannochloropsis gaditana
YTSGTTGPPK

SEQ ID NO: 58 - protein, 24 aa, Nannochloropsis gaditana
SITGRIKELIITAGGENIPPVLIE

SEQ ID NO: 59 - protein, 5 aa, Artificial Sequence
  motif III
  PEPTIDE (1)..(1): F or Y
  misc_feature (3)..(3): Xaa can be any naturally occurring amino acid
XGXSE

SEQ ID NO: 60 - protein, 5 aa, Nannochloropsis gaditana
LPLSH